(19) United States
(12) Patent Application Publication
(10) Pub. No.: US 2020/0224168 A1
(43) Pub. Date: Jul. 16, 2020

(54) COMPOSITIONS AND METHODS FOR ENHANCING MATURATION STATES OF HEALTHY AND DISEASED CARDIOMYOCYTES

(71) Applicant: University of Washington, Seattle, WA (US)

(72) Inventors: Deok-Ho KIM, Seattle, WA (US); Jesse MACADANGDANG, Seattle, WA (US); Alec S.T. SMITH, Seattle, WA (US); Hannele RUOHOLA-BAKER, Seattle, WA (US); Jason Wayne MIKLAS, Seattle, WA (US)

(73) Assignee: University of Washington, Seattle, WA (US)

(21) Appl. No.: 16/638,693

(22) PCT Filed: Aug. 16, 2018
(86) PCT No.: PCT/IB2018/056169

§ 371 Date: Feb. 12, 2020

Related U.S. Application Data
(60) Provisional application No. 62/546,438, filed on Aug. 16, 2017.

(51) Int. Cl. C12N 5/077; G01N 33/50

(52) U.S. Cl. CPC C12N 5/0657; G01N 33/502; C12N 2501/999; C12N 2510/00; C12N 2506/02; C12N 2506/45; C12N 2501/65

(57) ABSTRACT

The methods and compositions as disclosed herein describe the making of mature stem cell-derived cardiomyocytes for applications such as disease modeling, cardiotoxicity screening, drug screening and identification, among other uses. The methods involve physical and biochemical cues that promote a transition of stem cell-derived cardiomyocytes from a fetal phenotype to a more mature phenotype that more closely resembles that of adult cardiomyocytes.

### CROSS-REFERENCE TO RELATED APPLICATION

This application is a 35 U.S.C. § 371 National Phase Entry Application of International Application No. PCT/IB2018/056169 filed Aug. 16, 2018 which designates the U.S., and claims benefit under 35 U.S.C. § 119(e) of U.S. Provisional Application No. 62/546,438 filed Aug. 16, 2017, the contents of each of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Nov. 8, 2018, is named 034186-092680WOPT_SL.txt and is 5,144 bytes in size.

### FIELD OF THE INVENTION

The present disclosure relates to in vitro models for cardiac function and disease.

### BACKGROUND

Cardiovascular disease remains the leading cause of death for both men and women worldwide, with a rapidly growing impact on developing nations. Inherited cardiomyopathies are the major cause of heart disease in all age groups, including children and young, otherwise healthy adults. Human pluripotent stem cell-derived cardiomyocytes (hPSC-CMs) are a promising tool for studying cardiomyopathy because they vastly increase the accessibility to human cardiomyocytes that can express the full array of ion channels, protein isoforms, and genetic and metabolic machinery found in the human heart.

Directed differentiation of human pluripotent stem cells (hPSCs) into cardiomyocytes typically produces cells with structural, functional, and biochemical properties that most closely resemble those present in the fetal heart. However, there is an unmet need to develop hPSC-CMs that have an adult cardiomyocyte phenotype that comprises enhanced sarcomere development, enhanced contractile function, reduced beat rate, enhanced mitochondrial capacity, and a more mature transcriptome when compared with newly differentiated hPSCs. A major goal of current cardiac disease modeling efforts is to gain insight into the onset and progression of cardiomyopathies as a first step towards designing more effective therapeutic strategies. Prior to the methods described herein, adult-onset muscular disorders and cardiovascular diseases, such as Duchenne muscular dystrophy, have not been accurately modeled by hPSC-CMs. This highlights the need for improved stem cell differentiation and maturation methods their use in disease modelling and drug screening applications.

### SUMMARY OF THE INVENTION

Described herein are methods of engineering a developmental cardiac niche platform to produce mature stem cell-derived cardiomyocytes. The methods involve physical and biochemical cues that promote a transition of stem cell-derived cardiomyocytes from a fetal phenotype to a more mature phenotype that more closely resembles that of adult cardiomyocytes.

The approaches described herein provide more mature cardiomyocytes differentiated in vitro from stem cells, and thereby a platform for evaluating drugs for cardiotoxicity and screening to identify new drugs that modulate cardiomyocyte or cardiac function, among other uses. The ability to generate stem cells, e.g., induced pluripotent stem cells (iPS cells) from normal and diseased subjects, as well as the ability to genetically modify the stem cells prior to in vitro differentiation provides improved disease modeling and a platform for drug screening and identification.

Accordingly, in one aspect, described herein is a method of making stem cell-derived cardiomyocytes, the method comprising, contacting stem cell derived cardiomyocytes with: a nanopatterned substrate; thyroid hormone T3; and a Let7i microRNA.

In one embodiment, the nanopatterned substrate comprises a nanopatterned surface with a substantially parallel array of grooves and ridges.

In one embodiment, the dimensions of each groove or ridge are less than 1000 nanometers in length, width, or height.

In one embodiment, the grooves and ridges are about 800 nm wide, and the grooves are about 600 nm deep.

In one embodiment, the Let7i microRNA is expressed by the stem cell-derived cardiomyocyte.

In one embodiment, the step of contacting the cardiomyocytes with a Let7i microRNA comprises contacting the stem cell-derived cardiomyocytes with a viral vector.

In one embodiment, the stem cell-derived cardiomyocytes are human cardiomyocytes.

In one embodiment, the stem cell-derived cardiomyocytes are differentiated from an induced pluripotent stem cell (iPS cell) or an embryonic stem cell.

In one embodiment, the stem cell-derived cardiomyocytes are derived from a subject with a muscular disease or disorder.

In one embodiment, the stem cell-derived cardiomyocytes are genetically modified.

In one embodiment, the cardiomyocytes are contacted with nanopatterned substrate and thyroid hormone T3 after contacting with a vector encoding a Let7i microRNA.

In one embodiment, the resulting stem cell-derived cardiomyocytes have a more mature cardiomyocyte phenotype when compared with the stem cell-derived cardiomyocytes prior to contacting with the nanopatterned substrate, thyroid hormone T3, and Let7i microRNA.

In one embodiment, the nanopatterned substrate comprises a microelectrode array that permits electrical stimulation and/or measurement of cardiomyocyte electrophysiological properties.

In another aspect, described herein is a method of maturing stem cell-derived cardiomyocytes, the method comprising contacting stem cell derived cardiomyocytes with a nanopatterned substrate; thyroid hormone T3; and a Let7i microRNA.

In one embodiment, the nanopatterned substrate comprises a nanopatterned surface with a substantially parallel array of grooves and ridges.

In one embodiment, the dimensions of each groove or ridge are less than 1000 nanometers in length, width, or height.

In one embodiment, the grooves and ridges are about 800 nm wide, and the grooves are about 600 nm deep.

In one embodiment, the Let7i microRNA is expressed by the stem cell-derived cardiomyocyte.

In one embodiment, the step of contacting the cardiomyocytes with a Let7i microRNA comprises contacting the stem cell-derived cardiomyocytes with a viral vector.

In one embodiment, the stem cell-derived cardiomyocytes are human cardiomyocytes.

In one embodiment, the stem cell-derived cardiomyocytes are differentiated from an induced pluripotent stem cell (iPS cell) or an embryonic stem cell.

In one embodiment, the stem cell-derived cardiomyocytes are derived from a subject with a muscular disease or disorder.

In one embodiment, the stem cell-derived cardiomyocytes are genetically modified.

In one embodiment, the cardiomyocytes are contacted with nanopatterned substrate and thyroid hormone T3 after contacting with a vector encoding a Let7i microRNA.

In one embodiment, the resulting stem cell-derived cardiomyocytes have a more mature cardiomyocyte phenotype when compared with the stem cell-derived cardiomyocytes prior to contacting with the nanopatterned substrate, thyroid hormone T3, and Let7i microRNA.

In one embodiment, the nanopatterned substrate comprises a microelectrode array that permits electrical stimulation and/or measurement of cardiomyocyte electrophysiological properties.

In another aspect, described herein is a method of evaluating cardiotoxicity of an agent, the method comprising contacting stem cell-derived cardiomyocytes prepared by the methods described herein with the agent.

In one embodiment, the method comprises detecting at least one phenotypic characteristic of the cardiomyocytes.

In one embodiment, the agent is selected from the group consisting of a small molecule, an antibody, a peptide, a genome editing system and a nucleic acid.

In one embodiment, cardiotoxicity of an agent is indicated by the agent's effect on one or more of: cell viability, cell size, sarcomere length, organization of sarcomeres within a tissue, a biopotential or electrical property of a population of stem cell-derived cardiomyocytes, mitochondrial function, gene expression, beat rate, beat strength, and contractility.

In another aspect, described herein is an assay for identifying an agent that modulates a functional property of a cardiomyocyte, the assay comprising contacting a population of stem cell-derived cardiomyocytes prepared by the methods described herein with a candidate agent; and detecting at least one functional property of the cardiomyocytes, wherein detecting a change in at least one functional property of the cardiomyocytes identifies the agent as one that can modulate a functional property of a cardiomyocyte.

In one embodiment, the detecting step comprises detecting at least one of the following properties: cell viability, cell size, sarcomere length, organization of sarcomeres within a tissue, a biopotential or electrical property, mitochondrial function, gene expression, beat rate, beat strength, and contractility.

In another aspect, described herein is a disease model comprising a stem cell-derived cardiomyocyte prepared by the methods described herein, wherein the stem cell is derived from a subject with a muscular disease or disorder, or wherein the stem cell-derived cardiomyocyte or the stem cell from which it is derived is genetically modified such that the cardiomyocyte expresses a disease phenotype.

In one embodiment, the muscular disease or disorder has a phenotype with cardiac dysfunction.

In one embodiment, the muscular disease or disorder is characterized by adult onset of a cardiac disease phenotype.

In one embodiment, the muscular disease or disorder is Duchenne Muscular Dystrophy.

In another aspect, describe herein is a composition comprising stem cell-derived cardiomyocytes on a nanopatterned substrate, the composition further comprising thyroid hormone T3 and a Let7i microRNA.

In one embodiment, the stem cell-derived cardiomyocytes are derived from a subject with a muscular disease or disorder.

In one embodiment, the cardiomyocytes are human cardiomyocytes.

In one embodiment, the stem cell-derived cardiomyocytes, or the stem cells from which they are derived, are genetically modified such that the stem cell-derived cardiomyocytes exhibit a cardiac dysfunction phenotype.

In another aspect, described herein is a composition comprising cardiomyocytes made by contacting in vitro-differentiated cardiomyocytes with a nanopatterned substrate, thyroid hormone T3, and a Let7i microRNA wherein the cardiomyocytes have a more mature phenotype as compared with in vitro-differentiated cardiomyocytes that were not contacted with the nanopatterned substrate, thyroid hormone T3 and Let7i microRNA.

In one embodiment, the cardiomyocytes are derived from a subject with a muscular disease or disorder.

In another aspect, described herein is a composition of the in vitro-differentiated cardiomyocytes or the stem cells from which they are differentiated are genetically modified such that they exhibit a cardiac dysfunction phenotype.

In another aspect, described herein is a kit comprising stem cell-derived cardiomyocytes, a nanopatterned substrate, thyroid hormone T3, a vector encoding a Let7i microRNA, and packaging materials therefor.

In one embodiment, the kit comprises cell culture medium and instructions to permit preparation of mature in vitro differentiated cardiomyocytes from the stem cell-derived cardiomyocytes.

In one embodiment, the nanopatterned substrate comprises a nanopatterned surface with a substantially parallel array of grooves and ridges.

In one embodiment, the dimensions of each groove or ridge are less than 1000 nanometers in length, width, or height.

In one embodiment, the grooves and ridges are about 800 nm wide, and the grooves are about 600 nm deep.

In one embodiment, the stem cell-derived cardiomyocytes are human.

In one embodiment, the stem cell-derived cardiomyocytes are derived from a subject with a muscular disease or disorder.

In one embodiment, the cardiomyocytes are on the nanopatterned substrate.

In one embodiment, the stem cell-derived cardiomyocytes are frozen.

In one embodiment, the kit permits shipping at a temperature between room temperature and 4° C.

In another aspect, described herein is a method of making stem cell-derived cardiomyocytes, the method comprising, contacting stem cell derived cardiomyocytes with a nanopatterned substrate comprising a substantially parallel array of grooves and ridges, wherein the grooves and ridges are 800 nm wide, and the grooves are 600 nm deep; thyroid hormone T3; and a Let7i microRNA.

In another aspect, described herein is a composition comprising stem cell-derived cardiomyocytes on a nanopatterned substrate, the composition further comprising a nanopatterned substrate comprising a substantially parallel array of grooves and ridges, wherein the grooves and ridges are 800 nm wide, and the grooves are 600 nm deep; thyroid hormone T3; and a Let7i microRNA.

In one embodiment, described herein, is a method wherein the nanopatterned substrate comprises an elastomeric substrate that permits mechanical stimulation of the cardiomyocytes cultured thereupon, and wherein the method further comprises subjecting the cardiomyocytes to such mechanical stimulation.

In one embodiment, the compositions described herein, or the kit described herein, wherein the nanopatterned substrate comprises an elastomeric substrate that permits mechanical stimulation of cardiomyocytes cultured thereupon.

### BRIEF DESCRIPTION OF THE DRAWINGS

FIG. 1A-F shows ComboMat platform promotes a more mature transcriptional profile. FIG. 1A shows a schematic timeline of the ComboMat platform. Cardiomyocyte differentiation is achieved using a small molecule and human recombinant protein-based protocol. After cardiomyocyte production, hPSC-CMs are transfected with Let7i lentivirus, purified via metabolic challenge, and selected for viral transfection. High purity hPSC-CMs that express the viral vector are then plated on NPs and exposed to T3 for 3 weeks. FIG. 1B shows visualization of the Principle Component Analysis in three-dimensions. hPSC-CMs exposed to T3 cluster along PC2. The ComboMat group separates and is distinct from other combinations of the maturation stimuli. FIG. 1C shows net enrichment in cardiac maturation pathways of differentially expressed genes as a result of different treatments. FIG. 1D shows the gene ontology (GO) enrichment results of up-regulated genes in Let7i-NP-T3 combination treatment compared to EV-Flat control. Color gradient and bar lengths represent significance of enrichment. FIG. 1E-F shows the bubble plot of PCA loadings showing genes' contributions to the separation of RNAseq samples comparing EV-Flat to Let7i-NP-T3 (FIG. 1E) and fetal to adult cardiac cardiomyocytes (FIG. 1F). The Bubble plots in FIG. 1E and FIG. 1F highlight a similar up/down-regulation paten) of specific genes between the ComboMat-treated hPSC-CMs and adult CMs.

FIG. 2A-F shows the structural maturation of hPSC-CMs. FIG. 2A shows immunofluorescent images of hPSC-CMs exposed to the Combomat protocol or each cue in isolation. Red=alpha-actinin, Green=F-actin, Blue=DAPI. Insets show close-up of sarcomere structures within the hPSC-CMs. FIG. 2B shows TEM images of sarcomeres from hPSC-CMs exposed to the labeled cues of the Combomat platform. hPSC-CMs exposed to the Control conditions exhibit only Z-body formation whereas hPSC-CMs exposed to the ComboMat platform have much more defined Z-disc structures. FIG. 2C-2F shows the quantification of morphological changes for hPSC-CMs exposed to the Combomat protocol or each cue in isolation. FIG. 2C shows sarcomere lengths as measured via custom 2D Fourier transform image analysis. FIG. 2D shows box-and-whisker plots of Z-band widths as measured from the TEM images with the mean Z-band width marked with the red line. FIG. 2E shows a bar chart of binucleated cells that shows the ComboMat group is approaching physiological binucleation percentages. FIG. 2F shows cell area as measured from immunofluorescent images shows a clear hypertrophic response to the ComboMat platform. Bars represent averages ±S.E.M. *P<0.05.

FIG. 3A-G shows the electromechanical and metabolic maturation. FIG. 3A shows representative traces of contraction velocities averaged across an entire video frame for Control or ComboMat hPSC-CMs as measured via a custom CCQ method. Peak contraction velocity and peak relaxation velocity are shown. FIG. 3B shows quantification of peak contraction and relaxation velocities. FIG. 3C shows the contraction angle distribution histogram for Control or ComboMat hPSC-CMs. The line in the center of the histogram denotes the average angle of contraction while the length of the line indicates the degree to which the distribution is aligned with the average. FIG. 3D shows contraction anisotropic ratio (AR) of the magnitude of contraction in the longitudinal and transverse directions. FIG. 3E shows a representative field potential recording denoting the change in FPD between control and Combomat hPSC-CMs. FIG. 3F shows the quantification of FPD and FIG. 3G shows the beat rate expressed as Beats Per Minute for hPSC-CMs exposed to the Combmat platform or each cue in isolation. FIG. 3H shows representative OCR trace for fatty acid stress test using palmitate. FIG. 3I shows the quantification of maximum change in OCR following palmitate addition. Bars represent Bars represent averages ±S.E.M. *P<0.05.

FIG. 4A-E shows DMD mutant hPSC-CMs respond to ComboMat platform similarly as Normal control. FIG. 4A shows the Seahorse metabolic profile of DMD mutant hPSC-CMs exposed to Control or ComboMat conditions compared to Normal hPSC-CMs. FIG. 4B shows the quantification of the maximum change in OCR after FCCP addition. Both Normal and DMD mutant hPSC-CMs show an increase in maximum respiratory capacity when exposed to the ComboMat platform. FIG. 4C-D shows MEA-based measurements of electrophysiology. FIG. 4C shows beat rate represented as beats per minute for Normal or DMD mutant hPSC-CMs. FIG. 4D shows the average FPD for Normal or DMD mutant hPSC-CMs exposed to Control or ComboMat conditions. FIG. 4E shows the immunofluorescent images of DMD mutant hPSC-CMs exposed to Control (left) and ComboMat (right) conditions. Insets show zoomed-in portion of sarcomere structures in each condition. Bars represent averages ±S.E.M. *P<0.05.

FIG. 5A-F shows the endogenous DMD disease phenotype is exposed. FIG. 5A shows the representative field potential recordings from DMD mutant hPSC-CMs exposed to Control or ComboMat conditions with every-other beat interval annotated. FIG. 5B shows the change in beat interval (ΔBI) plotted for 30 consecutive beats for Normal and DMD mutant hPSC-CMs exposed to Control or ComboMat conditions. Mature DMD mutant hPSC-CMs exhibit a much greater instability in ΔBI compared to Normal hPSC-CMs. FIG. 5C shows the average percentage of beats with a ΔBI>250 ms for Normal or DMD mutant hPSC-CMs exposed to Control or ComboMat conditions. Immature Normal hPSC-CMs showed no incidence of a ΔBI>250 ms. FIG. 5D shows the mean ΔBI for entire 30 beat recording. Immature DMD mutant hPSC-CMs show no distinguishing disease phenotype on the MEAs whereas DMD mutant hPSC-CMs matured using the ComboMat platform are significantly different from the Normal counterparts. FIG. 5E shows representative Fura-2 Ca2+ imaging traces of DMD mutant hPSC-CMs exposed to Control or ComboMat conditions. FIG. 5F quantification of diastolic Ca²⁺ content in the cytosol. DMD mutant hPSC-CMs exhibit a significantly higher cytosolic Ca′ content when matured using the ComboMat platform. Bars represent averages ±S.E.M. *P<0.05.

FIG. 6A-F shows a DMD cardiomyopathy phenotypic drug screen using mature hPSC-CMs. FIG. 6A shows the drug library ranked by Z-score for reduction in cell death in response to osmotic stress. Chemical structure of specific compounds are also shown. FIG. 6B shows the quantification of baseline mean ΔBI for Normal and DMD mutant hPSC-CMs exposed to Control or ComboMat conditions. FIG. 6C-D shows dose-response curves in response to nitrendipine and sildenafil after 7 days for Normal and DMD mutant hPSC-CMs exposed to Control or ComboMat conditions. FIG. 6E shows measurement of mean ΔBI 48 hours after washout of nitrendipine. FIG. 6F shows representative Fura-2 Ca²⁺ traces for mature DMD mutant hPSC-CMs. FIG. 6G shows quantification of diastolic Ca²⁺ content with and without nitrendipine. Nitrendipine helps to reduce cytosolic Ca²⁺ load in the mature DMD mutant hPSC-CMs. Bars represent averages ±S.E.M. *P<0.05.

FIG. 7A-C shows dystrophin mutant analysis. FIG. 7A shows a schematic representation of primer design for DMD gene sequencing in both the Normal (SEQ ID NO:18) and DMD mutant (SEQ ID NO: 19) hPSC-CMs, which reveals the G deletion at base pair 263. FIG. 7B shows a chromograph of DMD gene around the CRISPR engineered deletion at bp263 in the DMD mutant hPSC-CMs. FIG. 7C shows predicted protein sequence in Normal (top) and DMD mutant (bottom) hiPSC-CMs suggests as early STOP codon as a result of the frame shift deletion in exon 1.

FIG. 8A-F shows patch clamp electrophysiology. FIG. 8A shows current-clamp electrophysiological measurements of hPSC-CMs. hPSC-CMs exposed to ComboMat conditions had significantly faster upstroke velocity but all other parameters (FIG. 8B-F) where not statistically different between Control and ComboMat hPSC-CMs. Bars represent averages ±S.E.M. *P<0.05. Up Slope=Upstroke velocity of action potential, RMP=resting membrane potential, APD90=action potential duration of 90% repolarization, Decay Tau=time constant of relaxation, Amplitude=amplitude of action potential, MDP=maximum diastolic potential.

FIG. 9A-E shows metabolic profiling of hPSC-CMs. FIG. 9A shows representative OCR profile in response to ATP synthase inhibitor oligomycin, uncoupler of electron transport and oxidative phosphorylation (FCCP), and electron transport chain blockers rotenone and antimycin during MitoStree assay. FIG. 9B shows quantification of basal and maximum respiratory capacity. FIG. 9C shows representative OCR trace for fatty acid stress test using palmitate. FIG. 9D-E shows quantification of OCR change in response to palmitate for Normal hiPSC-CMs. FIG. 9E shows representative OCR profile for fatty acid challenged and BSA control using ComboMat hPSC-CMs. Cells did not respond to the BSA carrier. Bars represent averages ±S.E.M. *P<0.05.

FIG. 10A-F shows that the full ComboMat necessary for disease state stratification. Arrhythmia metrics as outlined by Gilchrist et al.²⁵ There is no statistical difference between Normal and DMD mutant hPSC-CMs when the maturation cues are employed in isolation but when combined into the ComboMat protocol, there is a separation between the healthy Normal and diseased DMD mutant hPSC-CMs. Bars represent averages ±S.E.M. *P<0.05.

FIG. 11A-F shows the effect of long-term T3 exposure in hPSC-CMs. FIG. 11A-B show the effect of T3 on sarcomere length and cell area of hPSC-CMs measured at 7, 14, and 21 days post treatment as measured via immunohistochemistry. Combining NPs with T3 alone had a negative impact long term on sarcomere structure and hypertrophy. FIG. 11C-D show CCQ measurements of contraction (FIG. 11C) and relaxation velocities (FIG. 11D) of hPSC-CMs measured at 7, 14, and 21 days post treatment. Again combining NPs and T3 alone hindered contractile performance. FIG. 11E-F shows electrophysiological performance of hPSC-CMs exposed to Control or T3 conditions at 7, 14, and 21 days post treatment and measured via MEAs. FIG. 11E shows beat period peaked at D14 in T3 treated cells. FIG. 11F shows beat rate variability increased substantially in hPSC-CMs exposed to T3 alone. Bars represent averages ±S.E.M. *P<0.05.

FIG. 12A-E shows cardiomyocyte purification via metabolic challenge. FIG. 12A-B shows flow cytometry results for cTnT+ cells before (FIG. 12A) and after (FIG. 12B) lactate enrichment for a low cardiac purity starting population. FIG. 12C shows IgG isotype control flow cytometry results show no non-specific binding of cTnT antibody. FIG. 12D-E shows flow cytometry results for cTnT+ cells before (FIG. 12D) and after (FIG. 12E) lactate enrichment for a high cardiac purity starting population.

FIG. 13A-G shows nanotopographic size-dependent structural development of hPSC-CMs. FIG. 13A shows scanning electron micrographs of NP surfaces with various dimensions (top row), bright field images of hPSC-CMs on NP surfaces of various dimensions (middle row) and confocal immunofluorescent images of hPSC-CMs cultured on NPs of various dimensions (bottom row). FIG. 13B shows cell area and cellular anisotropic ratio of hPSC-CMs cultured on the labeled topographic dimensions. FIG. 13C shows cell monolayer density relative to initial seeding density of hPSC-CMs on the different topographic dimensions. FIG. 13D shows that relative cytoskeletal fiber alignment of hPSC-CMs of hPSC-CMs on various topographies. FIG. 13E-F shows Fluo-4 calcium imaging and time-to-peak Ca²⁺ measurements of hPSC-CMs between flat and 800 nm NPs. FIG. 13G shows RT-PCR expression levels of cardiac maturation markers of hPSC-CMs on 800 nm NPs compared to flat. Bars represent averages ±S.E.M. *P<0.05.

### DETAILED DESCRIPTION

The compositions and methods described herein are related, in part to the discovery that more mature stem cell-derived cardiomyocytes can be generated by a process including contacting the cells with a nanopatterned substrate, thyroid hormone (T3); and Let7i microRNA. Stem cell-derived cardiomyocytes with structural and functional phenotypes more characteristic of adult cardiomyocytes than standard methods. Also described herein are methods and compositions that establish an engineered developmental cardiac niche platform to produce hPSC-derived cardiomyocytes (hPSC-CMs) with enhanced sarcomere development, electrophysiology, contractile function, mitochondrial capacity, and a more mature transcriptome than standard methods.

The power of the platform is demonstrated herein, by application to cells with an engineered dystrophin gene mutation. When this developmental cardiac niche was applied to dystrophin mutant hPSC-CMs, a robust disease phenotype emerges, which was not previously observed in non-matured diseased hPSC-CMs that are not in contact with a nanopatterned substrate, thyroid hormone (T3), or Let7i microRNA. Matured dystrophin mutant hPSC-CMs exhibited a greater propensity for arrhythmia as measured via beat rate variability and further experiments indicated that the effect is most likely due to higher resting cytosolic calcium content. Further power is demonstrated herein, using a custom nanopatterned microelectrode array platform to screen hPSC-CM function within the developmental cardiac niche platform, where it is shown that for example, a calcium channel blocker, nitrendipine, mitigated arrhythmogenic behavior, and the assays correctly identified sildenafil as a false positive.

Taken together, the methods described herein, provide a developmental cardiac niche platform that permits robust hPSC-CM maturation, among other things, allowing for more accurate disease modeling, cardiotoxicity evaluation, and predictive drug screening.

### Definitions

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein, the term “contacting” when used in reference to a cell, encompasses both introducing an agent, surface, hormone, etc. to the cell in a manner that permits physical contact of the cell with the agent, surface, hormone etc., and introducing an element, such as a genetic construct or vector, that permits the expression of an agent, such as an miRNA, polypeptide, or other expression product in the cell. It should be understood that a cell genetically modified to express an agent, is “contacted” with the agent, as are the cell's progeny that express the agent.

As used herein, a “stem cell-derived cardiomyocyte” is a cardiomyocyte differentiated from a stem cell in culture, i.e., in vitro. Thus, while cardiomyocytes in vivo are ultimately derived from a stem cell, i.e., during development of a tissue or organism, a stem cell-derived cardiomyocyte as described herein has been created by in vitro differentiation from a stem cell. As used herein, a cell differentiated in vitro from a stem cell, e.g., an induced pluripotent stem (iPS) cell or embryonic stem cell (“ES cell” or “ESC”), is a “stem-cell derived cardiomyocyte” if it has, at a minimum, spontaneous beating or contraction, and expression of cardiac troponin T (cTnT). Methods for differentiating stem cells in vitro to cardiomyocytes are known in the art and described elsewhere herein.

The terms “stem cell” or “undifferentiated cell” as used herein, refer to a cell in an undifferentiated or partially differentiated state that has the property of self-renewal and has the developmental potential to differentiate into multiple cell types, without a specific implied meaning regarding developmental potential (i.e., totipotent, pluripotent, multipotent, etc.). A stem cell is capable of proliferation and giving rise to more such stem cells while maintaining its developmental potential. In theory, self-renewal can occur by either of two major mechanisms. Stem cells can divide asymmetrically, which is known as obligatory asymmetrical differentiation, with one daughter cell retaining the developmental potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also potentially retaining one or more cells with parental developmental potential. A differentiated cell may derive from a multipotent cell, which itself is derived from a multipotent cell, and so on. While each of these multipotent cells can be considered stem cells, the range of cell types each such stem cell can give rise to, i.e., their developmental potential, can vary considerably. Alternatively, some of the stem cells in a population can divide symmetrically into two stem cells, known as stochastic differentiation, thus maintaining some stem cells in the population as a whole, while other cells in the population give rise to differentiated progeny only. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art, and as used herein.

Exemplary stem cells include embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including methods for isolating and culturing them, may be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33 41; and U.S. Pat. Nos. 5,559,022, 5,672,346 and 5,827,735.

In the context of cell ontogeny, the term “differentiate”, or “differentiating” is a relative term that indicates a “differentiated cell” is a cell that has progressed further down a developmental pathway than its precursor cell. Thus in some embodiments, a stem cell as the term is defined herein, can differentiate to lineage-restricted precursor cells (such as a human cardiac progenitor cell or mid-primitive streak cardiogenic mesoderm progenitor cell), which in turn can differentiate into other types of precursor cells further down the pathway (such as a tissue specific precursor, such as a cardiomyocyte precursor), and then to an end-stage differentiated cell, which plays a characteristic role in a certain tissue type, and may or may not retain the capacity to proliferate further. Methods for in vitro differentiation of stem cells to cardiomyocytes are known in the art and/or described herein below. The differentiation status of a cell is generally determined by one or more of characteristic gene or marker expression pattern, metabolic activit(ies), and morphology.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers.

Stem cell-derived cardiomyocytes prepared by methods known in the art generally have a phenotype that is more similar to a fetal cardiomyocyte than a cardiomyocyte from an adult tissue. By culturing on a nanopatterned substrate and treating with thyroid hormone T3 and Let7i microRNA as described herein, stem cell-derived cardiomyocytes can be induced to a more mature phenotype, i.e., a phenotype that is more similar to a cardiomyocyte in a post-natal tissue or an adult tissue than the same cells cultured on a non-patterned substrate and without thyroid hormone T3 or Let7i microRNA treatment. It is also anticipated that culture of primary cardiomyocytes from fetal or even adult tissue could benefit from the conditions described herein, exhibiting a more mature phenotype or maintaining the cardiomyocyte phenotype for longer than in other conditions. By “more mature phenotype” in the context of cultured cardiomyocytes is meant that one or more phenotypic characteristics in one or more of sarcomere length, Z-band width, contraction velocity, field potential duration, upstroke velocity, cardiomyocyte diameter, cardiomyocyte length and mitochondrial capacity is increased (as defined herein) in a stem cell-derived cardiomyocyte cultured on a nanopatterned substrate with thyroid hormone T3 and Let7i treatment as described herein, relative to the same cardiomyocyte cultured on a non-patterned surface, without thyroid hormone T3 and Let7i treatment as described herein. In some embodiments, each of sarcomere length, Z-band width, contraction velocity, field potential duration, upstroke velocity, cardiomyocyte diameter, cardiomyocyte length and mitochondrial capacity is increased in a stem cell-derived cardiomyocyte cultured on a nanopatterned substrate with thyroid hormone T3 and Let7i treatment as described herein, relative to the same cardiomyocyte cultured on a non-patterned surface, without thyroid hormone T3 and Let7i treatment as described herein. In some embodiments, the increase in one or more of sarcomere length, Z-band width, contraction velocity, field potential duration, upstroke velocity, cardiomyocyte diameter, cardiomyocyte length and mitochondrial capacity occurs relative to a stem-cell derived cardiomyocyte cultured on a nanopatterned substrate without thyroid hormone T3 or Let7i miRNA, or, alternatively, relative to a stem-cell derived cardiomyocyte cultured on a nanopatterned surface with either, but not both of thyroid hormone T3 and Let7i miRNA.

Evidence of a “more mature phenotype” can also include upregulation or increased expression of one or more genes including Hair Growth Associated gene HR (a lysine demethylase and nuclear receptor corepressor), Krupel-like factor KLF9, cytochrome c oxidase subunit 6A2 (COX6A2), myosin light chain 2 (MYL2) and MYOM3 e.g., by at least 1.5 fold relative to their expression in stem cell-derived cardiomyocytes cultured on a flat surface, without Let7i miRNA or thyroid hormone T3, or by at least 10% relative to their expression in stem cell-derived cardiomyocytes cultured on a nanopatterned surface with either, but not both of thyroid hormone T3 and Let7i miRNA.

A “mature stem cell-derived cardiomyocyte” or “cardiomyocyte with an adult phenotype” as described herein can, when derived from a subject with an adult-onset muscular disease, or when derived from a stem cell modified to provide a disease model for a muscular disease with an adult-onset phenotype, present the disease phenotype of the adult onset muscular disease. For example, a stem cell-derived cardiomyocyte derived from a subject with Duchenne muscular dystrophy or from a stem cell modified to mutate the dystrophin gene can, when cultured under maturation-promoting conditions described herein (i.e., nanopatterned substrate, with thyroid hormone T3 and Let7i miRNA) express an arrhythmia evident by beat rate variability that is not seen when the same cells are cultured on a flat surface without thyroid hormone T3 or Let7i miRNA.

The term “reprogramming” as used herein refers to a process that alters or reverses the differentiation state of a differentiated cell (e.g. a somatic cell). Stated another way, reprogramming refers to a process of driving the differentiation of a cell backwards to a more undifferentiated or more primitive type of cell. The cell to be reprogrammed can be either partially or terminally differentiated prior to reprogramming. In some embodiments, reprogramming encompasses complete reversion of the differentiation state of a differentiated cell (e.g. a somatic cell) to a pluripotent state. In some embodiments, reprogramming also encompasses partial reversion of the differentiation state of a differentiated cell (e.g. a somatic cell) to a multipotent state. In some embodiments, reprogramming encompasses complete or partial reversion of the differentiation state of a differentiated cell (e.g. a somatic cell) to an undifferentiated cell. Reprogramming also encompasses partial reversion of the differentiation state of a somatic cell to a state that renders the cell more susceptible to complete reprogramming to a pluripotent state when subjected to additional manipulations.

Reprogramming involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

As used herein, the terms “hPSC cell” and “human pluripotent stem cell” are used interchangeably and refer to a pluripotent cell artificially derived from a differentiated somatic cell. hPSC cells are capable of self-renewal and differentiation into cell fate-committed stem cells, including cells of the cardiac lineages, as well as various types of mature cells.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell.

The term “substrate” as used herein refers in the broad sense to a composition comprising a biocompatible matrix, scaffold, or the like upon which a cell can be cultured. A cell capable of growing in monolayer or three-dimensional culture (adherent, as opposed to suspension culture) will generally attach to a substrate. Nanopatterned substrates as described herein provide structural cues that promote cardiomyocytes to assume morphological and functional properties that resemble cardiomyocytes in vivo. In certain embodiments, a substrate comprises synthetic or semi-synthetic materials. In certain embodiments, the substrate comprises a framework or support, such as a polymer scaffold.

The term “nanopatterned substrate” as used herein refers to a patterned surface with a parallel array of ridges and grooves that have a width and height less than 1000 nanometers. Patterning less than about 50-100 nanometers in depth or width is less likely than patterning with larger nanoscale dimensions to promote a more mature cardiomyocyte phenotype. Thus, depth/height of a groove can be 100 nanometers or more, or 150 nanometers or more, or 200 nanometers or more, or 250 nanometers or more, or 300 nanometers or more, or 350 nanometers or more, or 400 nanometers or more, or 450 nanometers or more, of 500 nanometers or more, or 550 nanometers or more, or 600 nanometers or more, or 650 nanometers or more, or 700 nanometers or more, or 750 nanometers or more, or 800 nanometers or more, or 850 nanometers or more, or 900 nanometers or more, or 950 nanometers or more, not to exceed 1000 nanometers. The width of a groove or ridge between grooves of a nanopattern on a nanopatterned substrate can be 100 nanometers or more, or 150 nanometers or more, or 200 nanometers or more, or 250 nanometers or more, or 300 nanometers or more, or 350 nanometers or more, or 400 nanometers or more, or 450 nanometers or more, or 500 nanometers or more, or 550 nanometers or more, or 600 nanometers or more, or 650 nanometers or more, or 700 nanometers or more, or 750 nanometers or more, or 800 nanometers or more, or 850 nanometers or more, or 900 nanometers or more, or 950 nanometers or more, not to exceed 1000 nanometers. In one embodiment, the depth of the grooves, width of the grooves, and width of the ridges between the grooves are all the same dimension, e.g., 100 nanometers or more, 150 nanometers or more, 200 nanometers or more, 250 nanometers or more, 300 nanometers or more, 350 nanometers or more, 400 nanometers or more, 450 nanometers or more, 500 nanometers or more, 550 nanometers or more, 600 nanometers or more, 650 nanometers or more, 700 nanometers or more, 750 nanometers or more, 800 nanometers or more, 850 nanometers or more, 900 nanometers or more, 950 nanometers or more, not to exceed 1000 nanometers. In another embodiment, the depth of the grooves, width of the grooves and width of the ridges between the grooves include, e.g., depth of 100, with groove width of 100, 150, 200, 250, 300, 350 400, 450, 500, 550, 600, 650, 700, 750, 800, 850, 900 or 950 nanometers and ridge width of 100, 150, 200, 250, 300, 350 400, 450, 500, 550, 600, 650, 700, 750, 800, 850, 900 or 950 nanometers. In another embodiment, the groove depth is 200 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 300 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 400 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 500 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 600 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 700 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 800 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 900 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. The dimensions provided here as examples are even multiples of 50 or 100 nm, but it is contemplated that other dimensions that are not even multiples of 50 or 100 nm can also provide benefit for the phenotype of stem cell-derived cardiomyocytes cultured on substrates with such nanopatterns.

It should be understood that relevant differences in nanopattern dimensions are those that result in a difference in cultured cardiomyocyte phenotype as described herein.

A nanopatterned substrate can be comprised of a polymer, hydrogel, polydimethylsiloxane, plastic, glass, resin, matrix, or any other material known in the art that permits the fabrication of nanopatterning and permits, either naturally or following surface treatment or coating, the attachment of stem cell derived cardiomyocytes. If desired to promote cardiomyocyte interaction, the nanopatterned substrate can be coated with an extracellular matrix protein such as fibronectin, collagen, laminin, or other extracellular matrix material known in the art.

The term “agent,” as used herein, means any compound or substance including, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogue, protein, antibody, peptide, aptamer, oligomer of nucleotides, amino acids, or carbohydrates including without limitation proteins, oligonucleotides, ribozymes, DNAzymes, glycoproteins, siRNAs, lipoproteins and modifications and combinations thereof. In certain embodiments, agents are small molecules comprising or consisting of chemical moieties including unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

As used herein, the term “small molecule” refers to a chemical agent which can include an organic or inorganic compound (including heterorganic and organometallic compounds) having a molecular weight less than about 5,000 grams per mole, an organic or inorganic compound having a molecular weight less than about 1,000 grams per mole, an organic or inorganic compound having a molecular weight less than about 500 grams per mole, and salts, esters, and other pharmaceutically acceptable forms of such compounds.

The term “cardiotoxicity” refers to the property of a drug or agent that inhibits one or more of cardiomyocyte viability, structure or function, including but not limited to contraction, biopotential or electrophysiological properties or rhythm thereof, or gene expression necessary for proper cardiac function.

As used herein, the terms “nucleic acid,” “polynucleotide,” and “oligonucleotide” generally refer to any polyribonucleotide or poly-deoxyribonucleotide, and includes unmodified RNA, unmodified DNA, modified RNA, and modified DNA. Polynucleotides include, without limitation, single- and double-stranded DNA and RNA polynucleotides. The term polynucleotide, as it is used herein, embraces chemically, enzymatically or metabolically modified forms of polynucleotides, as well as the naturally occurring chemical forms of DNA and RNA found in or characteristic of viruses and cells, including for example, simple (prokaryotic) and complex (eukaryotic) cells. A nucleic acid polynucleotide or oligonucleotide as described herein retains the ability to hybridize to its cognate complimentary strand.

Accordingly, as used herein, the terms “nucleic acid,” “polynucleotide,” and “oligonucleotide” also encompass primers and probes, as well as oligonucleotide fragments, and is generic to polydeoxyribonucleotides (containing 2-deoxy-D-ribose), to polyribonucleotides (containing D-ribose), and to any other type of polynucleotide which is an N-glycoside of a purine or pyrimidine base, or modified purine or pyrimidine bases (including, but not limited to, abasic sites). There is no intended distinction in length between the term “nucleic acid,” “polynucleotide,” and “oligonucleotide,” and these terms are used interchangeably. These terms refer only to the primary structure of the molecule. An oligonucleotide is not necessarily physically derived from any existing or natural sequence, but can be generated in any manner, including chemical synthesis, DNA replication, DNA amplification, in vitro transcription, reverse transcription or any combination thereof

As described herein, a “genetically modified cell” is a cell which either carries a heterologous genetic material or construct, or which comprises a genome that has been manipulated, e.g., by mutation, including but not limited to site-directed mutation. The introduction of a heterologous genetic material generally results in a change in gene or protein expression relative to an un-modified cell. Introduction of RNA can transiently promote expression of a foreign or heterologous product, as can the introduction of a vector that does not integrate or replicate within the cell. Introduction of a construct that integrates into a cell's genome or replicates with the cell's nucleic acid will be more stable through successive cell divisions. In one embodiment, genetic modification is in addition to or separate from the introduction of a construct or constructs that reprogram a somatic cell to a stem cell phenotype, such as an iPS cell phenotype. In another embodiment, the genetic modification is in addition to or separate from the introduction of a Let7i miRNA or a construct encoding one. Genetic modifications can include, but are not limited to the introduction of genetic material via viral vector or modification using CRISPR/Cas or similar system for site specific recombination.

The term “functional assay” as used herein refers to a test which assesses the properties of a cell, such as a cell's gene expression, metabolism, developmental state or maturity, among others, by measurement of a cellular activity. Functional assays include, for example, measurement of cell viability (e.g., by dye exclusion, nutrient uptake and/or conversion, metabolite production, etc.), measurement of electrical potential or other electrophysiological property, measurement of contraction strength, rate or rhythm, measurement of mitochondrial function, etc.

The term “disease model” as used herein refers to an animal or cell culture system that recapitulates one or more aspects of a human disease. An animal model of disease, e.g., a mouse or rat model, will often fairly closely resemble the human disease due to one or more mutations to disease-related genes or the introduction of a heterologous construct that expresses one or more disease-related products. Cell culture models of human disease can include cells from a human subject with the disease, or human or other cells modified to express or interfere with expression of one or more disease-related genes. As but one example, hPSCs derived from a human with Duchenne muscular dystrophy or from a cell in which the dystrophin gene has been inactivated, when differentiated to cardiomyocytes and treated as described herein to promote maturity can provide a cell culture model of DMD cardiomyopathy marked by a pronounced arrhythmia.

A “marker” as used herein refers to one or more characteristics that contribute to or are associated with the phenotype of a given cell. Markers can be used for selection of cells comprising characteristics of interest. Markers will vary with specific cells, and can be characteristics, whether morphological, functional or biochemical, that are particular to a cell or tissue type, or to a disease state or phenotype and include both extracellular and intracellular molecules, e.g., proteins, RNAs, glycosylation patterns, etc. expressed or exhibited by the cell or tissue. In some embodiments, such markers are proteins, including, but not limited to proteins that possess an epitope for an antibody or other binding molecule available in the art. Other markers can include peptides, lipids, polysaccharides, nucleic acids and steroids, among others. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, the ability to differentiate along particular lineages, contraction, beating, etc. Markers can be detected by any appropriate method available to one of skill in the art.

As used herein, the terms “electrophysiology,” or “biopotential,” refer to the electrical properties of a cell. Specifically, these terms describe the characteristics of ion channel behavior for the flow of ionic currents through cells and the electrical signals transmitted by, e.g., cell-cell contact. Biopotential or electrophysiological properties can be measured by a number of techniques, including but not limited to whole cell patch clamp electrophysiological recording (automated or manual), microelectrode arrays, calcium imaging, optical mapping, or xCelligence™ real time cell analysis (Acea Biosciences, Inc., San Diego, Calif.).

“Electromechanical stimulation” is any change in potential produced by a substrate in contact with a cell or population of cells and/or further produces a mechanical motion or stimulus to the cells. The electrical and mechanical stimuli can be produced by an instrumented substrate at the same time (in phase) or at different times (out of phase). Non-limiting examples of mechanical stimuli include stretch, changes in rigidity, phase changing agents, pressure, vibration, or any other type of mechanical stimuli known in the art.

As used herein, the term “instrumented,” when used in reference to a substrate, refers to a substrate or device adapted or designed to perform specified functions such as measuring, testing, controlling, or stimulating. An example of an instrumented substrate is a nanopatterned microelectrode array.

A “microelectrode array,” “multielectrode array,” or “MEA” is a device with an array of micro-sized or nano-sized wire electrodes, that measure biopotentials of cells in contact with the electrode array and/or can provide electrical stimuli to the cells in contact with the array. The electrodes of an MEA are typically composed of indium tin oxide, titanium, or gold but can be composed of other types of conductive material or a composition comprising conductive and non-conductive materials. An MEA is used for in vitro studies to determine or manipulate, for example, the field potentials of a tissue or an engineered tissue (e.g. stem cell-derived cardiomyocytes in contact with a nanopatterned substrate).

The terms “decrease,” “reduced,” “reduction,” “decrease,” or “inhibit” as used herein generally refer to a decrease by a statistically significant amount. However, for avoidance of doubt, “reduced”, “reduction” or “decrease” or “inhibit” mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased,” “increase,” or “enhance,” or “activate” as used herein generally refer to an increase by a statically significant amount. However, for the avoidance of doubt, the terms “increased”, “increase” or “enhance” or “activate” mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase or more, relative to a reference level.

As used herein, the term “modulates” refers to an effect including increasing or decreasing a given parameter as those terms are defined herein.

As used herein, the term “functional property,” as applied to a cardiomyocyte or culture of cardiomyocytes refers to any of the parameters described herein as measures of cardiomyocyte function or mature cardiomyocyte function. A “change in functional property” is indicated by a statistically significant increase or decrease in a functional property.

As used herein, a “muscular disease or disorder” is one that adversely affects normal muscle function as either a primary effect of the disease or disorder or as a result of the disease or disorder's effect on other systems that impact muscle function. Cardiac diseases or disorders necessarily impact the proper function of the heart muscle, and include, but are not limited to cardiac arrhythmias, cardiomyopathies (e.g. hypertrophic and dilated), long QT syndromes, arrhythmogenic right ventricular dysplasia (ARVD), catecholaminergic polymorphic ventricular tachycardia (CPVT), or Barth syndrome. Duchenne muscular dystrophy affects cardiac muscle function in late stages.

The term “statistically significant” or “significantly” refers to statistical significance and generally means a change of two standard deviations (2SD) relative to a reference. The term refers to statistical evidence that there is a difference. It is defined as the probability of making a decision to reject the null hypothesis when the null hypothesis is actually true.

The term “contractility” as used herein and refers to the behavior of muscle cells, including cardiomyocytes, whereby they contract, either alone or, more often, in groups. Contractility can be measured in terms of the rate of contraction or relaxation and, for example, the force of contraction. The contractility of a plurality of cells is measured by biophysical and biomechanical properties of the force transmission. Contractility is measured using phase-contrast microscopy of monolayers or multiple-layers of said cardiac cells and computational analysis, which is calibrated with direct force measurement using force transducers.

The term “tissue” refers to a group or layer of similarly specialized cells which together perform certain special functions. The term “tissue-specific” refers to a source or defining characteristic of cells from a specific tissue.

As used herein, the phrase “cardiovascular condition, disease or disorder” is intended to include all disorders characterized by insufficient, undesired or abnormal cardiac muscular function, e.g., arrhythmia, ischemic heart disease, hypertensive heart disease and pulmonary hypertensive heart disease, congenital heart disease and any condition which leads to failure of the heart musculature in a subject, particularly a human subject. Insufficient or abnormal cardiac muscular function can be the result of disease, injury and/or aging.

The articles “a” and “an” are used herein to refer to one or to more than one (e.g., to at least one) of the grammatical object of the article. By way of example, “an element” means one element or more than one element.

Other than in the operating examples, or where otherwise indicated, all numbers expressing dimensions, quantities of ingredients or reaction conditions used herein should be understood as modified in all instances by the term “about.” The term “about” when used in connection with percentages can mean±1%. When used in connection with nanopattern dimensions, the term “about” refers to variations within the resolution achievable for a given pattern. For example, if the resolution achievable for pattern generation is within ±50 nm, a dimension of 600 nanometers should be understood to encompass 550 nanometers to 650 nanometers. One of ordinary skill in the art will understand that different methods of generating nanopatterns will have different resolution, and will understand what the resolution is for a given method.

As used herein, the term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

The term “consisting essentially of” means “including principally, but not necessary solely at least one”, and as such, is intended to mean a “selection of one or more, and in any combination.” Stated another way, other elements can be included in the description of the composition, method or respective component thereof provided the other elements are limited to those that do not materially affect the basic and novel characteristic(s) of the invention (“consisting essentially of”). This applies equally to steps within a described method as well as compositions and components therein.

The term “consisting of” as used herein as used in reference to the inventions, compositions, methods, and respective components thereof, is intended to be exclusive of any element not deemed an essential element to the component, composition or method.

Cardiac Muscle Function and Muscular Diseases

There are many types of human muscle cells that can be derived from a stem cell using the methods and compositions described herein.

Muscle cells are soft tissue cells that contain protein filaments of actin and myosin that facilitate contraction of the muscle to perform various cellular, tissue, and organ-level functions. Muscle cells are required for the motion of limbs, but also include movement of food through the digestive system, beating of the heart, vascular constriction and dilation that regulates blood pressure, uterine contractions during birth, focusing of the iris for normal vision, and the constriction of the airway, among others.

The force produced by muscles is generated by the movement of myosin protein heads across actin filaments during a contraction. A series of signaling events take place within the muscle cell to produce a contraction that in general includes: 1) stimulation of electrical impulses of a nearby muscle cell and/or neuron; 2) the influx of intracellular calcium ions that trigger release of calcium from the sarcoplasmic reticulum of the cell; 3) calcium activation of calmodulin; 4) phosphorylation of myosin by a kinase in the presence of ATP (e.g. myosin light chain kinase, MLCK); and 5) “cross-bridge” formation between the myosin heads and actin filaments. These signaling events are important for muscle contractions to occur normally.

Muscular dystrophies such as Duchenne/Becker Muscular Dystrophy, and Limb-Girdle Muscular Dystrophies are the most common skeletal muscle disease in pediatric patients, and are for example, caused by genetic mutations in genes including dystrophin and its associated molecules. The principal symptoms of these degenerative muscle disorders include: progressive muscular wasting and muscle weakness, poor balance, frequent falls, walking difficulty, waddling gait, calf pain, limited range of movement, muscle contractures, drooping eyelids (ptosis), gonadal atrophy, scoliosis (curvature of the spine), and inability to walk. Often there is muscle necrosis which leads to high amounts of the muscle protein creatine kinase present in the blood.

Duchenne Muscular Dystrophy (DMD) is a hereditary (X-linked) progressive degeneration of muscles and is caused by the lack of a gene product, the cytoskeletal protein dystrophin, and affects approximately one in 3,500 male births. Lack of dystrophin is caused by mutation of the gene encoding this muscle fiber protein. Dystrophin associates with a large complex of membrane-bound proteins, termed the dystrophin glycoprotein complex (DGC). The loss of dystrophin, and the associated DGC, results in compromised structural integrity of the muscle plasma membrane, producing damaging cycles of muscle necrosis and regeneration. After constant damage to the muscles muscle cells are eventually replaced by non-contractile fibrous or fat tissues. The skeletal muscles of DMD patients undergo slow progressive damage which leads to the disease symptoms.

Skeletal muscles, diaphragm, and cardiac muscles can be affected. DMD is an example of a skeletal muscle disorder that also results in severe cardiovascular dysfunction. Patients with DMD often die from cardiac arrhythmias or sudden death. This is due to the progressive failure of cardiac muscle function in parallel with skeletal muscle function. However, the failure of the cardiac muscle in DMD patients occurs later in the disease and the cardiac onset generally begins in adulthood.

Cardiac electrophysiological and contractile function is a tightly controlled process. When ion channel regulation or contractile function is disrupted in a cardiac cell or tissue, this can result in cardiac arrhythmias that can sometimes be deadly. A number of diseases affect cardiac muscle, including but not limited to cardiac arrhythmias, cardiomyopathies (e.g. hypertrophic and dilated), long QT syndromes, arrhythmogenic right ventricular dysplasia (ARVD), catecholaminergic polymorphic ventricular tachycardia (CPVT), or Barth syndrome.

Stem cell-derived cardiomyocytes have been used to model these diseases and disease phenotypes (Itzhaki et al., Nature. 2011; Moretti et al., NEJM. 2010; Ma et al., European Heart Journal. 2012; Kim et al. Nature. 2013; Wang and McCain et al. Nature Medicine. 2014; Jung et al. EMBO Molecular Medicine. 2012). However, the functional maturity of hPSC-CMs in existing models is generally lacking and has not been well-controlled or remained consistent across normal and diseased hPSC-CMs. There is a need for quality control standards and methods for the maturation of hPSC-CMs. Reliable methods of hPSC-CM maturation are required, for example, to ensure that the disease phenotype represents, where appropriate, adult-onset of cardiac disease and disorders in order to identify therapeutics to treat these diseases and disorders effectively.

Apart from disease modeling, cardiotoxicity of drugs or agents for the treatment of diseases or disorders not generally related to cardiac function is a common reason for failure of investigative drugs. Where existing cell cultures of cardiomyocytes tend to have a more fetal or immature phenotype, they do not accurately reflect, likely in vivo effects of drugs on cardiac tissues. The methods and cell culture platforms described herein provide benefits in the evaluation of cardiotoxicity in this context as well. The methods and compositions provided herein allow for the generation of disease phenotypes such as DMD cardiomyopathy and can be applied to other cardiac diseases and disorders that have an adult-onset of the phenotype. The methods and compositions described herein produce hPSC-CMs that are functionally mature, with a more adult phenotype based on a number of functional assays described herein.

It is contemplated that the methods described herein can be applied to other types of muscle cells (e.g. skeletal muscle) using cell-specific microRNAs in the place of Let7i microRNA to mature stem cells from a fetal-like state to an adult form of the muscle cell or tissue. One example of a muscle tissue that is crucial for movement is skeletal muscle. Skeletal muscle is a striated muscle tissue that is under voluntary control of the somatic nervous system. It is contemplated that the methods described herein could be applied to disease modelling of muscular diseases such as DMD by stem cell derived-skeletal muscle cells.

It is further contemplated that the methods described herein can be applied to smooth muscle. Non-limiting examples of smooth muscles include those in the iris of the eye, bronchioles of the lung, laryngeal muscles (vocal cords), muscular layers of the stomach, esophagus, small and large intestine of the gastrointestinal tract, ureter, detrusor muscle of the urinary bladder, uterine myometrium, penis, or prostate gland. It is contemplated that analogous methods of stem cell-derived muscle cell maturation to those described herein can be applied to the maturation of stem cell derived-smooth muscle cells.

Preparation of Stem Cell-Derived Cardiomyocytes

Thus, in one embodiment, cardiomyocytes for use in the compositions and methods described herein can be obtained from cardiac tissue that is, primary cardiomyocytes. As a first matter, while the disclosure focuses on the use of cardiomyocytes differentiated in vitro from stem cells, the cues for maintaining or promoting cardiomyocyte phenotype provided by culture on a nanopatterned substrate, with thyroid hormone T3 and Let7i miRNA can benefit primary cells.

Also described herein are methods of producing stem cell-derived cardiomyocytes beginning with somatic cells derived from a subject, patient or donor. The somatic cells are reprogrammed to induced pluripotent stem cells (iPS cells, iPSCs), which are then differentiated to cardiomyocytes. Thus, described herein are methods of reprogramming somatic cells to iPS cells, and also described herein are methods of differentiating iPS cells to stem cell-derived cardiomyocytes (e.g. human pluripotent stem cell-derived cardiomyocytes, hPSC-CMs). Methods for differentiating embryonic stem cells to cardiomyocytes are known in the art and largely analogous to those for differentiating iPS cells to cardiomyocytes.

Sources of Somatic Cells for Reprogramming into iPS Cells:

Stem cell derived-cardiomyocytes are produced from a donor cell induced to a pluripotent stem cell phenotype that is then differentiated along the cardiomyocyte lineage. Without limitations, iPS cells can be produced from any animal in addition to humans. In some embodiments, the cell donor is a mammal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. The cell donor, patient, or subject can include any of the subset of the foregoing as appropriate for a given use. In certain embodiments of the aspects described herein, the subject is a mammal, e.g., a primate, e.g., a human.

iPS cells can also be produced from donor stem cells. Exemplary stem cells include adult stem cells, neural stem cells, liver stem cells, muscle stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including method for isolating and culturing them, can be found in, among other places, Embryonic Stem Cells, Methods and Protocols, Turksen, ed., Humana Press, 2002; Weisman et al., Annu. Rev. Cell. Dev. Biol. 17:387 403; Pittinger et al., Science, 284:143 47, 1999; Animal Cell Culture, Masters, ed., Oxford University Press, 2000; Jackson et al., PNAS 96(25):14482 86, 1999; Zuk et al., Tissue Engineering, 7:211 228, 2001 (“Zuk et al.”); Atala et al., particularly Chapters 33-41; and U.S. Pat. Nos. 5,559,022, 5,672,346 and 5,827,735.

More often, iPS cells will be generated from differentiated donor cells including nucleated somatic cells including, but not limited to fibroblasts, stromal cells, muscle cells or cells of any of a wide number of tissues in the adult organism. Indeed, the Examples described herein use human cells isolated from urine and reprogrammed into iPS cells (See the Examples and e.g. Zhou et al., J. Urology, 2012, Guan et al., Stem Cell Research, 2014). Donor cells can be obtained from the subject by a skin biopsy, urine sample, or by drawing blood, among other methods.

In some embodiments, the iPS cells can be reprogrammed from cardiac cells, e.g., cardiac fibroblasts or from ventricular cardiomyocytes obtained from a subject, e.g., a mammalian subject including a human subject. A mixture of cells from a suitable source of cardiac tissue can be harvested from a mammalian donor by methods known in the art. The heart tissue is dissociated and cells plated in culture. Cardiac fibroblasts will adhere to the surface of the culture dish, permit collecting of the cardiac fibroblasts to reprogram to iPS cells.

Reprogramming of Somatic Cells to iPS Cells:

Methods of reprogramming differentiated cells to iPS cells are well known in the art and generally involve forced expression of Oct¾, Sox2, Klf4, and c-Myc in the cells, although numerous variations are known in the art. For example, in the Examples provided herein, a polycistronic lentiviral vector encoding human Oct3/4, Sox2, Klf4, and c-Myc was used to reprogram cells collected from clean-catch urine samples into iPSCs. hiPSCs are cultured, expanded and passaged according to the methods described herein or other conditions favorable to cell viability and maintenance of the undifferentiated, pluripotent phenotype. hiPSCs are maintained, for example in hypoxic conditions (e.g. 37° C., 5% CO₂, 5% O₂).

Without limitations, iPS cells can also be generated using other methods, including, but not limited to non-viral methods, use of polycistronic vectors, mRNA species, miRNAs, and proteins, including methods described in, for example, International Patent Applications WO2010/019569, WO2009/149233, WO2009/093022, WO2010/022194, WO2009/101084, WO2008/038148, WO2010/059806, WO2010/057614, WO2010/056831, WO2010/050626, WO2010/033906, WO2009/126250, WO2009/143421, WO2009/140655, WO2009/133971, WO2009/101407, WO2009/091659, WO2009/086425, WO2009/079007, WO2009/058413, WO2009/032456, WO2009/032194, WO2008/103462, JP4411362, EP2128245, and U.S. Patent Applications US2004/0072343, US2009/0253203, US2010/0112693, US2010/07542, US2009/0246875, US2009/0203141, US2010/00625343, US2009/0269763, and US2010/059806, each of which are incorporated herein in their entirety by reference. While any tissue can provide source cells for generating iPS cells to differentiate into cardiomyocytes, it is contemplated that cells derived from cardiac tissue may have benefits, for example, epigenetic memory, that permit enhanced generation of mature cardiomyocytes when used in the methods described herein to generate iPS cell-derived cardiomyocytes.

Differentiating Stem Cells to Stem Cell-Derived Cardiomyocytes:

As described herein, iPSCs are dissociated into single cells using a cell dissociation reagent (e.g. trypsin or ethylenediaminetetraacetic acid) and plated on matrix-coated (e.g. Matrigel™ coated) plates in stem cell culture medium (See Current Protocols in Stem Cell Biology. 2:1C.2.1-1C2.16, 2007) in preparation for differentiation to cardiomyocytes. Other extracellular matrix protein surface treatments can be used for standard monolayer culture of the iPS cells. Non-limiting examples of surface treatments of culture dishes include proteins or mixtures thereof such as gelatin, collagen, fibronectin, etc.

To prepare iPS cells for differentiation to cardiomyocytes, the iPS cells are seeded at high density (250,000 cells/mm² or more). Once a monolayer has formed, cells can be differentiated to cardiomyocytes as described in the Examples herein, or, for example, as described by Macadangdang J, et al., Cell. Mol. Bioeng. 8: 320-332 (2015). Other approaches known in the art for differentiating pluripotent stem cells (iPS or ES cells) to cardiomyocytes can also be used.

In some embodiments, the iPS cells are genetically modified after or during the pluripotent state. For example, disease model cell lines, (e.g. DMD mutant human iPS cells) can be generated using CRISPR-Cas technology to create an isogenic pair from a normal parental cell line (e.g. UC3-4). Other approaches for genetically modifying iPS or donor cells can also be used, including but not limited to the introduction of viral vectors, liposome-mediated transfections, microinjection, etc. Genetic modifications can include both changes that increase expression of one or more factors, or, conversely, that inactivate or otherwise inhibit expression of one or more factors.

In the CRISPR-Cas approach, guide sequences targeting a muscle-specific exon of the target dystrophin gene are used to mutate the gene. Mutation is then confirmed by sequencing methods known in the art. The disease model cell lines are then maintained in the same manner and conditions as the normal or wild type cell lines as described herein.

Thyroid Hormone and Let7i microRNA Treatment for Stem Cell-Derived Cardiomyocyte Maturation

The methods and compositions described herein include the delivery of Let7i microRNA and thyroid hormone T3 for the maturation of stem cell derived-cardiomyocytes.

Delivery of Thyroid Hormone and Let7i miRNA:

Let7i miRNA

In the context of delivering Let7i miRNA, the term “contacting,” “delivering” or “delivery” is intended to encompass both delivery of a Let7i miRNA from outside the cell, and delivery from within the cell. For example, miRNAs can be introduced from outside the cell, e.g., in a lipid complex (e.g., liposomes). Alternatively, the miRNA can be delivered by expression within the cell from an exogenous construct, e.g., a viral or other expression vector. Such a construct can be episomal or stably integrated within the cell's genome. In one embodiment, the step of contacting a cardiomyocyte with a Let7i miRNA comprises the use of cardiomyocytes that stably express Let7i from a construct. It is also contemplated, that expression of the endogenous Let7i miRNA gene sequence can be upregulated to effect Let7i miRNA delivery.

The combination of a nanopatterned substrate, thyroid hormone T3 and Let7i miRNA provides efficient maturation of stem cell-derived cardiomyocytes, and the timing of these various stimuli can influence the maturation. In one embodiment, in vitro-differentiated cardiomyocytes are infected with a Let7i miRNA-encoding viral vector at a point where they have, at a minimum, formed beating monolayers on a non-patterned substrate and express cardiac troponin T. Timing can vary, but in one embodiment, this is at about 15 days post-induction of differentiation. Then thyroid hormone T3 is added to the medium and the cells are introduced to a nanopatterned substrate. While not wishing to be bound by theory, it is thought that one function of the Let7i miRNA is to at least partially inhibit the effects of thyroid hormone T3, which was found to have beneficial effects on maturation, but to include detrimental effects after long term exposure. One might expect that in this situation, the Let7i miRNA would provide the best benefit if administered after, rather than before the thyroid hormone. However, the expression of Let7i miRNA from a viral vector involves some delay in establishing expression of the transgene; it is contemplated that other means of Let7i miRNA delivery that provide the miRNA more quickly may be effective if administered concurrently with or after the thyroid hormone.

Thyroid Hormone T3:

Thyroid hormone T3 or Triiodothyronine (also referred to herein as, simply “T3”), is a tyrosine-based hormone produced and released by the thyroid gland. T3 is primarily responsible in vivo for regulation of metabolism, growth and development, body temperature, and heart rate. T3 binds to nuclear hormone receptors within their target cell where the thyroid hormone receptors then bind to and activate transcription via response elements in the regulatory regions of genes involved in metabolism, cell growth and development. For most cell types, T3 typically results in an increase in the basal metabolic rate, which can overall increase the body's oxygen and energy consumption.

In the heart, thyroid hormone T3 increases the heart rate and force of contraction, which increases cardiac output, by increasing myosin and β-adrenergic receptor levels in the myocardium. Furthermore, the action of T3 can shorten the time between the QRS complexes on an electrocardiogram (which has been demonstrated by animal models with hyperthyroidism). T3 can also improve the calcium handling of stem cell derived-cardiomyocytes. Following birth, the serum levels of thyroid hormone T3 immediately spike. This allows for further maturation and improved contractile function of the infant heart.

Thyroid hormone T3 at a concentration of 20 ng/ml is demonstrated to provide beneficial effects on cardiomyocyte maturation in the Examples provided herein. However, the concentration can vary, e.g., between 1 ng/ml and 50 ng/ml, or any concentration therebetween. Further, while thyroid hormone T3 is the metabolically active form of the hormone, it is contemplated that thyroid hormone T4, which is deiodinated by cellular deiodinase enzymes to the active T3 form, can be used, provided that the cells have the capacity to deiodinate the T4 form. The deiodinase enzymes involved in deiodination of T4 contain selenium, such that medium containing selenium can promote this activity. Analogs of thyroid hormone T3 that promote thyroid hormone receptor activity are also contemplated for use in the cardiomyocyte maturation process described herein. Examples include 3,3′,5′-triiodothyroacetic acid (Triac), 3,3′,5,5′-tetraiodothyroacetic acid (Tetrac), 3,5-diiodothyropropionic acid (DIPTA) and dextro(D)-T4 (choloxin). Analogs, and how they are assayed for activity are described in, for example, Shoemaker et al., Endocrin. Pract. 18: 954-964 (2012), and Groeneweg et al., Mol. Cell. Endocrinol. 458: 82-90 (2017).

It is contemplated that T3 can be provided through use of conditioned medium from the culture of T3-producing thyroid follicular cells, e.g., the cell line PCCL3 (Palmero et al. Mol. Cell Endocrinol. 376: 12-22 (2013).

Let-7i miRNA:

miRNAs are transcribed by RNA polymerase II as part of capped and polyadenylated primary transcripts (pii-miRNAs). The primary transcript is cleaved by the Drosha ribonuclease III enzyme to produce an approximately 70-nt stem loop precursor miRNA (pre-miRNA). The pre-miRNA is further cleaved by the cytoplasmic Dicer ribonuclease to generate the mature miRNA and antisense miRNA (sometimes referred to as miRNA star or miRNA*) products. The mature miRNA is incorporated into an RNA-induced silencing complex (RISC), which binds target mRNAs through imperfect base pairing with the miRNA, and most generally results in translational inhibition or destabilization of the target mRNA.

The Let7 family of microRNAs in vertebrates are conserved sequences across species with temporal expression during many developmental processes to promote terminal differentiation. The Let7 family of microRNAs have many targets which include, for example, protooncogenes, cell cycle regulators, regulators of cell proliferation, apoptosis, and immunity, such as RAS, HMGA2, cyclin A2, CDC34, Aurora A and B kinases, E2F5, CDK8, CDC25A, CDK6, Casp3, Bcl2, Map3k1, Cdk5, cytokines, toll-like receptors, among others.

Let-7i regulates, among other genes, the expression of the Hair Growth Associated gene HR, that acts as a transcriptional co-repressor of many nuclear receptors, including the thyroid hormone receptor.

Members of the Let-7 family are characterized by a highly conserved 18 nucleotide core sequence, with members Let-7a, Let-7b, Let-7c, Let-7a and Let-7i sharing exact sequence identity in the 16 nucleotide sequence UGAGGUAGUAGUUUGU. Human Let-7i is encoded at chromosome 12q14.1. The human Let-7i precursor sequence is (5′-3′) (SEQ ID NO: 5):

CUGGCUGAGGUAGUAGUUUGUGCUGUUGGUCG
GGUUGUGACAUUGCCCGCUGUGGAGAUAACUG
CGCAAGCUACUGCCUUGCUAG

The human Let-7i mature form molecule is a duplex with the sequence (5′-3′) (SEQ ID NO: 6):

UGAGGUAGUAGUUUGUGCU

and its complement (SEQ ID NO: 7).

The sequences for the mature forms of Let-7a to Let-7i shown below show that the most significant variation relative to other members of the family is at the 3′ end of the sequence of the mature form. As such, changes to the Let-7i molecule at or near the 3′ end might be expected to affect the specificity or function of the Let-7i molecule in cardiomyocyte maturation. In contrast, changes towards the 5′ end or center of the mature form sequence would be more likely to be tolerated in terms of permitting: function of the molecule iii cardiomyocyte maturation. Let-7 miRNAs tend to undergo imperfect base pair hybridization interactions with their target sequences, meaning that 100% complementarily with target RNA is not necessary. As such, it is contemplated that sequence variation of 5 or fewer, e.g., 4 or fewer, 3 or fewer, 2 or fewer or a single nucleotide (preferably in the 5′ or more central regions of the sequence) can be tolerated while retaining function iii cardiomyocyte maturation. Where there is more than one sequence difference relative to the human mature form sequence, it can be preferable that the successive differences are not located next to each other—for example, if there are 3, 4 or 5 differences, it can be preferable that they not be at contiguous nucleotides. In the end, what matters most for a Let-7i miRNA in the context of the methods and compositions described herein is that the miRNA promotes a more mature cardiomyocyte phenotype in stem-cell derived cardiomyocytes, either alone, or in combination with thyroid hormone T3 and a nanopatterned substrate. It is a straightforward matter to assay a given sequence variant for such activity using the methods described herein.

As used herein, then, “a Let-7i iniRNA” is a duplex RNA molecule with the 5′-3′ sequence of UGAGGUAGUAGUUUGUGCU (SEQ ID NO: 6) and its complement (SEQ ID NO: 7), or a molecule that differs, as described above, at 5 or fewer nucleotide positions, that promotes a more mature cardiomyocyte phenotype (i.e., promotes cardiomyocyte maturation) as that term is defined herein. Let-7i miRNAs can be expressed, e.g., from a vector, as a pri-mRNA or as a pre-miRNA that is processed in the cell to the mature form duplex. Alternatively, it is contemplated that mature form duplex can be introduced to cells, e.g., via lipid complexes or other direct delivery form, if so desired. It is also contemplated that direct delivery of a precursor miRNA or a hairpin molecule bearing the miRNA sequence can be employed; in this situation, the cell's processing enzymes are used to generate the mature form miRNA in the cell.

TABLE 1
Human Mature Let-7 microRNA Sequences.

| Mature | | Corresponding | |
|---|---|---|---|
| miRNA | Mature miRNA Sequence | precursor | SEQ |
| Name | (5′ to 3′) | microRNA(s) | ID NO |
| let-7a | UGAGGUAGUAGGUUGUAUAGUU | let-7a-1; let-7a-2; | 10 |
| | | let-7a-3; let-7a-4; | |
| let-7b | UGAGGUAGUAGGUUGUGUGGUU | let-7b | 11 |
| let-7c | UGAGGUAGUAGGUUGUAUGGUU | let-7c | 12 |
| let-7d | AGAGGUAGUAGGUUGCAUAGU | let-7d; let-7d-v1 | 13 |
| let-7e | UGAGGUAGGAGGUUGUAUAGU | let-7e | 14 |
| let-7f | UGAGGUAGUAGAUUGUAUAGUU | let-7f-1; let-7f-2-1; | 15 |
| | | let-7f-2-2 | |
| let-7g | UGAGGUAGUAGUUUGUACAGU | let-7g | 16 |
| let-7i | UGAGGUAGUAGUUUGUGCU | let-7i | 17 |

Where a Let-7i miRNA is delivered directly, i.e., not expressed in the cell, the miRNA can include modifications that enhance, for example, the stability of the molecule and/or its interactions with target molecules. Modifications can be, for example, to the nucleobase or to the backbone. Non-limiting examples of modified nucleobases include 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine, and 3-deazaguanine and 3-deazaadenine.

MicroRNA backbone modifications can include, but are not limited to deoxyribonucleic acid (DNA, SEQ ID NO: 1-5), peptide nucleic acid (PNA), morpholinos, locked nucleic acids (LNA), glycol nucleic acids (GNA), threose nucleic acids (TNA), or other xeno nucleic acid (XNA) forms known in the art. For any nucleobase or backbone modification, it should be understood that the promotion of cardiomyocyte maturation or a more mature cardiomyocyte phenotype as described herein is necessary for the modified miRNA to be used in the methods and compositions described herein.

A Let7i miRNA vector may also contain a selectable marker that permits positive or negative selection of stem cell-derived cardiomyocytes that express the miRNA. In most cases the selectable marker will impart resistance of the cells to an agent such as an antibiotic. Non-limiting examples of selectable markers include resistance genes for puromycin, ampicillin, kanamycin, geneticin, neomycin, bleomycin, methotrexate, G418 sulfate, among others. The selection can occur by adding the appropriate amount of the selection agent to the cell culture medium to identify successfully transduced stem cell-derived cardiomyocytes that express the Let7i miRNA.

Fabrication and Use of Biomimetic Nanopatterned Substrates for Stem Cell-Derived Muscle Cell Maturation

For compositions and methods as described herein that use biomimetic nanopatterned substrates for culture and maturation of stem cell-derived cardiomyocytes, the nanopatterned substrates can be produced by any method known in the art that is appropriate for the substrate material used. In some embodiments, nanopatterning can be introduced to a substrate as described, for example, by Kim et al., Proc. Natl. Acad. Sci. U.S.A. 107: 565-570 (2010), by Macadangdang et al., J. Vis. Exp. 88: 50039 (2014), or as described in U.S. Pat. No. 9,994,812, each of which are incorporated herein by reference in their entireties. A nanopatterned array parallel grooves and ridges can be produced using a process including capillary force lithography, nanoindentation, e-beam lithography, electrospinning or other methods known to those of ordinary skill in the art. In some embodiments, capillary force lithography is used.

In some embodiments, the polymer substrate is composed of a biocompatible hydrogel compatible with thermal or UV-based curing methods or a material that permits the use of capillary force lithography. In some embodiments, the polymer substrate is composed of PEG, PUA, PLGA, PMMA, PUA-PGMA, or a chemical variant thereof. In some embodiments, the polymer substrate comprises a UV curable hydrogel polymer, a thermosensitive hydrogel polymer or a polymer produced by solvent evaporation. In some embodiments, the thermosensitive polymer is PNIPAM.

Anisotropically nanofabricated substrata can be produced, for example, with 800 nanometer wide ridges and grooves that are 600 nanometers in depth using UV-assisted capillary force lithography (Macadangdang J, et al., Cell Mol Bioeng. 2015, WO2013151755A1). Other dimensions can be used. Patterning less than about 50-100 nanometers in depth or width is less likely than patterning with larger nanoscale dimensions to promote a more mature cardiomyocyte phenotype. Thus, depth/height of a groove can be 100 nanometers or more, or 150 nanometers or more, or 200 nanometers or more, or 250 nanometers or more, or 300 nanometers or more, or 350 nanometers or more, or 400 nanometers or more, or 450 nanometers or more, of 500 nanometers or more, or 550 nanometers or more, or 600 nanometers or more, or 650 nanometers or more, or 700 nanometers or more, or 750 nanometers or more, or 800 nanometers or more, or 850 nanometers or more, or 900 nanometers or more, or 950 nanometers or more, not to exceed 1000 nanometers. The width of a groove or ridge between grooves of a nanopattern on a nanopatterned substrate can be 100 nanometers or more, or 150 nanometers or more, or 200 nanometers or more, or 250 nanometers or more, or 300 nanometers or more, or 350 nanometers or more, or 400 nanometers or more, or 450 nanometers or more, or 500 nanometers or more, or 550 nanometers or more, or 600 nanometers or more, or 650 nanometers or more, or 700 nanometers or more, or 750 nanometers or more, or 800 nanometers or more, or 850 nanometers or more, or 900 nanometers or more, or 950 nanometers or more, not to exceed 1000 nanometers. In one embodiment, the depth of the grooves, width of the grooves, and width of the ridges between the grooves are all the same dimension, e.g., 100 nanometers or more, 150 nanometers or more, 200 nanometers or more, 250 nanometers or more, 300 nanometers or more, 350 nanometers or more, 400 nanometers or more, 450 nanometers or more, 500 nanometers or more, 550 nanometers or more, 600 nanometers or more, 650 nanometers or more, 700 nanometers or more, 750 nanometers or more, 800 nanometers or more, 850 nanometers or more, 900 nanometers or more, 950 nanometers or more, not to exceed 1000 nanometers. In another embodiment, the depth of the grooves, width of the grooves and width of the ridges between the grooves include, e.g., depth of 100, with groove width of 100, 150, 200, 250, 300, 350 400, 450, 500, 550, 600, 650, 700, 750, 800, 850, 900 or 950 nanometers and ridge width of 100, 150, 200, 250, 300, 350 400, 450, 500, 550, 600, 650, 700, 750, 800, 850, 900 or 950 nanometers. In another embodiment, the groove depth is 200 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 300 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 400 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 500 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 600 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 700 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 800 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. In another embodiment, the groove depth is 900 nanometers, and the groove width is 100, or 150, or 200, or 250, or 300, or 350, or 400, or 450, or 500, or 550, or 600, or 650, or 700, or 750, or 800, or 850, or 900, or 950 nanometers, and the ridge width is 100, or 150, or 200, or 250, or 300, or 350, or 400, or 450, or 500, or 550, or 600, or 650, or 700, or 750, or 800, or 850, or 900, or 950 nanometers. The dimensions provided here as examples are even multiples of 50 or 100 nm, but it is contemplated that other dimensions that are not even multiples of 50 or 100 nm can also provide benefit for the phenotype of stem cell-derived cardiomyocytes cultured on substrates with such nanopatterns.

In some embodiments, the nanopatterned substrate comprises a nanotextured array of parallel grooves and ridges on one or both sides of a substantially planar substrate. In some embodiments, the cardiomyocytes or stem cells can be present on one or both sides of the nanopatterned substrate.

The grooves and ridges described and demonstrated in the Examples provided herein are generally rectangular where, for example, the side of a groove meets the ridge between grooves at a perpendicular angle, or similarly where the side of a groove meets the bottom of a groove. However, the patterning of the extracellular matrix in vivo is not comprised of features with perpendicular or rectangular transitions. Thus, it is also contemplated herein that the transitions between ridges and grooves can be, for example, sinusoidal. What is important is that the patterning have substantially parallel grooves and ridges of nanoscale dimensions of height and width as described herein. The term parallel is used in its standard meaning, i.e., that the adjacent grooves or ridges do not intersect in the span of the culture surface. However, it is recognized that some degree of imperfection, or even some degree of designed intersection of adjacent grooves can be tolerated without sacrificing the benefit of the texture. Thus, while absolute numbers may not fully capture the degree of non-parallel or intersecting grooves and ridges that can be tolerated, it should be understood that groves and ridges are “substantially parallel” if, over the span of culture surface where cardiomyocyte structure or function will be measured in use of the nanopatterned substrate, two adjacent grooves or ridges do not intersect, or intersect at most 20% of the time and only at an acute angle less than 20 degrees when viewed down the length of the grooves or ridges. Overall, the topographic features chosen should permit sarcomere formation of normal or wild-type stem cell-derived cardiomyocytes such that they present ordered Z-bands and H-zones of the sliding actin and myosin filaments. The Z-band width in cells, an indicator of myofibril bundling, is significantly larger in mature cardiomyocytes, as compared with non-patterned cells alone. Sarcomere formation can be verified, for example, by immunohistochemistry of stem cell-derived cardiomyocytes.

Nanopatterned substrates can be treated to promote cell adhesion, for example, by coating with an extracellular matrix protein preparation or other biocompatible surface treatment. In some embodiments, a biocompatible surface treatment can include, for example, poly-L-lysine, poly-D-lysine, poly-orinithine, or an extracellular matrix protein such as vitronectin, erythronectin. gelatin, collagen type I, collagen type IV, fibronectin, fibronectin domains, laminin, or engineered extracellular matrix proteins or peptides. Engineered proteins can be, for example, peptide segments including CS1, RGD, domains in extracellular matrix proteins that bind to integrin receptors, and others commonly known to person of ordinary skill in the art.

In some embodiments, the nanopatterned substrate is directly fabricated on a multielectrode array for field potential recordings of the stem cell-derived cardiomyocytes. In some embodiments, the nanopatterned substrate is fabricated with nano or microposts that allow for deflection of the posts by a cardiomyocyte to measure contractility. In some embodiments, the nanopatterned substrate is fabricated on a laminar thin film that deflects when cardiomyocytes contract or are electrically simulated. In some embodiments, the nanopatterned substrate is fabricated on a laminar thin film that deflects when cardiomyocytes contract or are electrically simulated. In some embodiments, the nanopatterned substrate is fabricated onto a device that allows for stretch of the cardiomyocytes (e.g. silicone or hydrogels). While other materials can be used, an example of an elastomeric substrate that permits mechanical stimulation and stretching of cardiomyocytes to further improve their maturity is polydimethylsiloxane (PDMS). Other examples of platforms on which the nanopatterned substrates can be fabricated include those described in Du et al., Nat Commun. 8: 400 (2017), U.S. Pat. No. 9,994,812, U.S. 20120027807 A1, and U.S. 20120004716 A1.

As noted above, subjecting cardiomyocytes to mechanical stimulation by stretching, e.g., subjecting them to repeated cycles of stretch and relaxation can promote a more mature phenotype. While the combination of nanopatterned substrate, thyroid hormone T3 and Let7i treatment provides superior cardiomyocyte maturation, it is contemplated that this maturation can be even further promoted by combination of these cues with other stimuli or conditions. Non-limiting examples include, as noted above, the application of mechanical stretch/relaxation cycles, and the addition of fatty acids to the culture medium. Thus, in one embodiment, cardiomyocytes subjected to the nanopatterned substrate/thyroid hormone T3/Let7i treatment described herein can further be subjected to stretching, e.g., through use of an elastomeric nanopatterned substrate engaged with a device for application of stretch/relaxation cycles, to further promote a more mature phenotype as the term is described herein. Assays and measurements as described herein can provide a measure of further matured phenotype with this additional stimulus.

In another embodiment, culture in the presence of fatty acids, including, but not limited to palmitate, oleic acid, and linoleic acid, can further promote a more mature phenotype as the term is described herein. As non-limiting examples, the addition of palmitate conjugated to bovine serum albumin (BSA) (e.g., approximately 50 □M), oleic acid (e.g., approximately 15 □g/ml), or linoleic acid (e.g., approximately 10 □g/ml), or combinations thereof, to the basal medium can further promote a more mature phenotype as described herein.

In another embodiment, the nanopatterned substrate/thyroid hormone T3/Let7i treatment can be combined with both mechanical stimulation and fatty acid exposure to further promote a more mature phenotype as described herein.

Functional Characterization of Stem Cell-Derived Cardiomyocytes

Stem cell-derived cardiomyocytes matured under the conditions described herein permit evaluation of the response of mature cardiomyocytes to various treatments or stimuli. In various embodiments, quantifiable parameters of stem cell-derived cardiomyocytes can include contractile force, contractility, altered contraction, frequency of contraction, contraction duration, contraction stamina, cardiomyocyte size, sarcomere organization, length, circumference, structure, multinucleate status, metabolic respiratory capacity, oxygen consumption, electrophysiological and biophysical parameters. In some embodiments, quantifiable parameters include survival and/or division or regeneration of the stem cell-derived cardiomyocytes.

While most parameters will provide a quantitative readout, in some instances a semi-quantitative or qualitative result will be acceptable. Readouts can include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods to provide useful values.

Measurements of various parameters useful for evaluating cardiomyocyte status or function are described in the following.

Immunoassays:

Standard methods in immunology known in the art and not specifically described are generally followed as in Stites et al. (Eds.), Basic and Clinical Immunology, 8th Ed., Appleton & Lange, Norwalk, Conn. (1994); and Mishell and Shigi (Eds.), Selected Methods in Cellular Immunology, W. H. Freeman and Co., New York (1980).

In general, immunoassays are employed to assess a specimen for cell surface or intracellular markers, depending upon how the cell is prepared for assay. Immunocytochemical assays are well known to those skilled in the art. Both polyclonal and monoclonal antibodies can be used in such assays. Where appropriate, other immunoassays, such as enzyme-linked immunosorbent assays (ELISAs), radioimmunoassays and flow cytometry can be used to detect cell type specific markers.

Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771; and 5,281,521 as well as Sambrook et al., Molecular Cloning: A Laboratory Manual, Cold Springs Harbor, N.Y., 1989, among others.

Non-limiting examples of cardiac-specific markers one can assay for using immuno-based methods include cardiac troponin T, cardiac troponin-C, tropomyosin, caveolin-3, GATA-4, myosin heavy chain, myosin light chain-2a, myosin light chain-2v, ryanodine receptor, atrial natriuretic factor, among others.

Sarcomere Organization:

The methods described herein include the use of sarcomere morphology and structural characterization of actin and myosin filaments as a measure of stem cell-derived cardiomyocyte differentiation and maturation. A sarcomere is defined as the segment between two neighboring Z-lines (or Z-discs, or Z bodies) of a muscle cell that appear in micrographs as a series of dark lines. The Z-line acts as an anchoring point for the actin and myosin filaments for muscle contraction. A cardiac sarcomere comprises (i) thick myosin protein filaments with myosin heads are nearly perpendicular to the filaments, (ii) thin actin filaments, and (iii) titin filaments. Between each Z-line is an I band of thin filaments that are not superimposed by thick myosin filaments. Other structural (and variable) features of cardiac muscle cell organization include the A band, which is the entire length of a single thick myosin filament. The H zone is the zone of the thick filaments that are not superimposed by actin thin filaments. The M-line is formed by the cross-connecting elements of the cellular cytoskeleton.

Actin filaments and titin molecules are cross-linked in the Z-disc via the Z-line protein alpha-actinin Within the laminar tissue of the heart, the alpha-actinin is perpendicular to the actin and myosin filaments. Because the structure of the cardiac sarcomeres is highly ordered, one with ordinary skill in the art would recognize these proteins (actin, myosin, alpha-actinin, titin) and their arrangement in tissues or collections of cultured cells as markers to identify mature muscle cells and tissues. Developing cardiac cells undergo “sarcomerogenesis,” which creates new sarcomere units within the cell. The degree of sarcomere organization provides a measure of cardiomyocyte maturity.

Immunofluorescence assays for myosin, actin, cTnT, tropomyosin, among others and electron microscopy can be used to identify and measure sarcomere structures.

Immunofluorescent images can be quantified for sarcomere alignment, pattern strength, and sarcomere length. This can be accomplished using a scanning gradient and Fourier transform script to determine the position of the proteins that. This is done by taking each image broken into several small segments for individual analysis. Using a directional derivative, the image gradient for each segment can be calculated to determine the local alignment of sarcomeres. The pattern strength can be determined by calculating the maximum peaks of one-dimensional Fourier transforms in the direction of the gradient. The lengths of sarcomeres can be calculated by measuring the intensity profiles of the sarcomeres along this same gradient direction. The frequency at which the intensity profiles cross their mean allows an accurate calculation of local sarcomere length within each image segment. This analysis allows for unbiased subcellular-resolution mapping of sarcomere patterning, even in cells lacking the alignment cues provided by nanotopography.

Cellular morphology can be used to identify structurally mature stem cell-derived cardiomyocytes. Non-limiting examples of morphological and structural parameters include but are not limited to sarcomere length, Z-band width, binucleation percentages, nuclear eccentricity, cell area, and cell aspect ratio.

Alternatively, qualitative analysis of sarcomeric structure can also be used. For one with ordinary skill in the art, the standard for an adult cardiomyocyte phenotype is that the alpha-actin is about 90 degrees to the actin filaments. A fetal cardiomyocyte does not express alpha-actinin, or the alpha actinin structure is disordered from the sarcomere structure described herein.

Electrophysiology:

Mature cardiomyocytes have functional ion channels that produce electrical potentials that produce signals between cardiomyocytes, allowing for the synchronization of cardiac muscle contraction. The electrical function of stem cell-derived cardiomyocytes can be measured by a variety of methods. Non-limiting examples of such methods include whole cell patch clamp (manual or automated), multielectrode arrays, calcium imaging and optical mapping among others. Stem cell-derived cardiomyocytes can be electrically stimulated during whole cell current clamp or multielectrode array recordings to produce an electrical or contractile response. Furthermore, stem cell-derived cardiomyocytes can be genetically modified, for example, to express a channel rhodopsin that allows for optical stimulation of the cells.

Measurement of field potentials and biopotentials stem cell-derived cardiomyocytes can be used to determine differentiation stage and cell maturity. Without limitations, the following parameters can be used to determine electrophysiological function of stem cell-derived cardiomyocytes: change in FPD, quantification of FPD, beat frequency, beats per minute, upstroke velocity, resting membrane potential, amplitude of action potential, maximum diastolic potential, time constant of relaxation, action potential duration of 90% repolarization, interspike interval, change in beat interval, current density, activation and inactivation kinetics, among others.

During a disease state, the electrophysiological function of cardiomyocytes can be compromised, and this can be recapitulated by disease models using cardiomyocytes matured as described herein. For example, stem cell-derived cardiomyocytes used to model a cardiac arrhythmia such as long QT syndrome, may exhibit prolonged FPD and APD when compared with normal stem cell-derived cardiomyocytes.

Metabolic Assays:

Adult cardiomyocytes have been shown to have enhanced cellular metabolism compared with fetal cardiomyocytes marked by increased mitochondrial function and spare respiratory capacity. Metabolic assays can be used to determine the differentiation stage and cell maturity of the stem cell-derived cardiomyocytes as described herein. Non-limiting examples of metabolic assays include cellular bioenergetics assays (e.g. Seahorse Bioscience XF Extracellular Flux Analyzer), and oxygen consumption tests.

More specifically, cellular metabolism can be quantified by oxygen consumption rate (OCR), OCR trace during a fatty acid stress test, maximum change in OCR, maximum change in OCR after FCCP addition, and maximum respiratory capacity among other parameters.

Furthermore, a metabolic challenge or lactate enrichment assay can provide a measure of stem cell-derived cardiomyocyte maturity or a measure of the effects of various treatments of such cells. Mammalian cells generally use glucose as their main energy source. However, cardiomyocytes are capable of energy production from different sources such as lactate or fatty acids. In some embodiments, lactate-supplemented and glucose-depleted culture medium, or the ability of cells to use lactate or fatty acids as an energy source is useful to identify mature stem cell-derived cardiomyocytes and variations in their function.

Methods to Measure Cardiac Contractility:

Contractility is typically measured by video tracking methods. Functional outputs such as contraction magnitude, velocity, and angle are output as a vector field for each video frame and can be averaged spatially or temporally. For video tracking methods, contraction (or systole) of the stem cell-derived cardiomyocytes is considered to be the point in time and space where the cell or cardiac tissue is at the shortest length. Relaxation (diastole) is considered to be the point in time or space where the cell or cardiac tissue is at the largest length. These parameters are determined by using a reference frame within the video and tracking the motion of the cardiomyocyte or cardiac tissue using the reference frame as a guide.

In addition to video tracking, impedimetric measurements can also be performed. For example, the stem cell-derived cardiomyocytes described herein can have contractility or beat rate measurements determined by xCelligence™ real time cell analysis (Acea Biosciences, Inc., San Diego, Calif.).

An important parameter of stem cell-derived cardiomyocyte function is beat rate. The frequency of the contraction, beat rate, change in beat interval (ΔBI), or beat period, can be used to determine stem cell differentiation stage, stem cell-derived cardiomyocyte maturity, and the effects of a given treatment on such rate. The beat rate is typically elevated in fetal cardiomyocytes and is reduced as cardiomyocytes develop. During disease states the change in beat rate can be variable and lack a constant frequency due to electrophysiological or structural instability. Without limitations, contractile parameters can also include contraction velocity, relaxation velocity, contraction angle distribution, or contraction anisotropic ratio.

Disease Models

DMD is but one of a number of diseases affecting cardiac function for which stem cell-derived cardiomyocytes prepared and matured as described herein can be adapted to model. A number of cardiac disease models have been described in which the cardiac cells are differentiated from iPS cells, derived from subjects with various cardiac diseases. See, e.g., Ebert and Svendsen, Nat. Rev. Drug Discov 9: 367-372 (2010).

Cardiac diseases that can be modeled using stem cell-derived cardiomyocytes prepared and matured as described herein include those discussed, for example, in Smith et al., Biotechnol. Adv. 35: 77-94 (2017). More specifically, such diseases include ion channelopathies, such as Long QT Syndrome, for which a model using cardiomyocytes derived from iPS cells is described by Moretti et al., New Engl. J. Med. 363: 1397-1409 (2010). Other models include a model for the LQT1 subtype described by Egashira et al., Cardiovasc. Res. 95: 419-429 (2012), models for the LQT2 subtype described by Itzhaki et al., Nature 471: 225-229 (2011), Lahti et al., Dis. Model Mech. 5: 220-230 (2012), and Matsa et al., Eur. Heart J. 32: 952-962 (2011), and models for the LQT3 subtype described by Ma et al., Int. J. Cardiol. 168: 5277-5286 (2013) and Terrenoire et al., J. Gen. Physiol. 141: 61-72 (2013). A model giving rise to both LQT3 and Brugada Syndrome subtypes is described by Davis et al., Circulation 125: 3079-3091 (2012), and a model for LTQ8 from Timothy Syndrome patients is described by Yazawa et al., J. Cardiovasc. Transl. Res. 6: 1-9 (2013).

Further cardiac diseases that can be modeled using cardiac cells differentiated from patients with such disease include the channelopathy catecholaminergic polymorphic ventricular tachycardia (CPVT). Models for this disease based on iPS cells differentiated to cardiomyocytes include those described by Itzhaki et al., J. Am. Coll. Cardiol. 60: 990-1000 (2012), Fatima et al., Cell Physiol. Biochem. 28: 579-592 (2011), Jung et al., EMBO Mol. Med. 4: 180-191 (2012) DiPasquale et al., Cell Death Dis. 4: e843 (2013), and Paavola et al., Europace: European pacing, arrhythmias, and cardiac electrophysiology: journal of the working groups on cardiac pacing, arrhythmias, and cardiac cellular electrophysiology of the European Society of Cardiology (2015) for CPVT1, and Novak et al., Rambam Maimonides Med. J. 3: e0015 (2012) for CPVT2.

Other disorders that have been modeled using iPS cell differentiation from subjects with diseases include hypertrophic cardiomyopathy (HCM) (Carvajal-Vergara et al., Nature 2010 465: 808-812 (2010) and Lan et al., Cell Stem Cell 12: 101-113 (2013)), and familial dilated cardiomyopathy (DCM) (Sun et al., Sci Transl. Med. 4: 130ra47 (2012), Siu et al., Aging 4: 803-822 (2012), Tse et al., Hum. Mol. Genet. 22: 1395-1403 (2013), Pompe disease (Huang et al., Hum. Mol. Genet. 20: 4851-4864 (2011), Friedrich's ataxia (FRDA) (Hick et al., Dis. Model. Mech. 6: 608-621 (2013), Barth Syndrome (Wang et al., Nat. Med. 20: 616-623 (2014)), arrhythmogenic right ventricular dysplasia/cardiomyopathy (ARVD/C) Calkins, Circulation Journal: official Journal of the Japanese Circulation Society 79: 901-913 (2015), Caspi et al., Circulation Cardiovascular Genetics 6: 557-568 (2013), Kim et al., Nature 494: 105-110 (2013), Malik and Rao, Meth. Mol. Biol. 997: 23-33 (2013).

Any or all of the disease models described above that use cardiac cells differentiated from iPS cells derived from subjects with the various diseases can benefit from the methods described herein for preparing and maturing stem cell-derived cardiomyocytes.

Drug Screening Platforms Using Stem Cell-Derived Cardiomyocytes

The matured cardiomyocytes prepared as described herein provide a platform for the study or evaluation of the likely effects of known or experimental drugs on cardiomyocytes or cardiac tissue in vivo. This can be used to evaluate drugs to be used for treating non-cardiac indications for possible cardiac side-effects or cardiotoxicity. Alternatively, by screening with, for example, a library or collection of potential drugs or agents, cardiomyocytes prepared and matured as described herein can also be used to identify new drugs with beneficial effects on cardiomyocyte or cardiac function. Cardiomyocytes derived from normal donor cells can provide useful information in both situations, and cardiomyocytes derived from donors with a cardiac or other disease, or derived from cells engineered to mimic a cardiac disease or disorder can be very useful in identifying new drugs or agents to treat such diseases. In either instance, the evaluation of functional or structural parameters as described herein or as known in the art can be informative with regard to the effects of a given agent. In general, such assays comprise contacting cardiomyocytes prepared and matured as described herein with an agent and measuring one or more parameters of the cardiomyocytes described herein as an indicator of the agent's effect(s). Where effects are observed, dose responses can also be evaluated by varying the concentration of the agent and/or the duration of contacting. One benefit of the matured cardiomyocytes as described herein is that they maintain their mature phenotype for an extended period in culture (weeks or more, under optimal conditions) relative to less mature cultured cardiomyocytes. This can also permit the collection of data on, for example, long term, low level exposure to an agent that would not be possible for cardiomyocytes in another platform.

Accordingly, stem cell-derived cardiomyocytes prepared and matured as described herein can be used to identify an agent or evaluate an agent for its effect on parameters such as expression of markers, cell viability, sarcomere arrangement, contractility, electrophysiological responses, beat rate, or other parameters described herein or known in the art.

In some embodiments, stem cell-derived cardiomyocytes can be used in assays to screen agents, selected from small molecules, nucleic acids or analogues thereof, aptamers; proteins or polypeptides or analogues or fragments thereof, among other agents for effects, detrimental or beneficial, on the cells. To the extent that cardiomyocytes prepared and matured as described herein can recapitulate or mimic the effects of drugs on cardiac tissue, it is also contemplated that cardiomyocytes prepared and matured as described herein can be used to screen for an agent that counters the cardiac side effect of another drug, useful for a non-cardiac indication.

In some embodiments, the agent is an agent of interest including known and unknown compounds that encompass numerous chemical classes, primarily organic molecules, which may include organometallic molecules, inorganic molecules, genetic sequences, etc. An important aspect of the use of stem cell-derived cardiomyocytes as described herein is to evaluate candidate drugs, including toxicity testing; and the like. Candidate agents also include organic molecules comprising functional groups necessary for structural interactions, particularly hydrogen bonding, and typically include amine, carbonyl, hydroxyl or carboxyl groups, frequently more than one of such functional chemical groups. The candidate agents often comprise cyclic carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof

Also included as agents are pharmacologically active drugs, genetically active molecules, etc. Compounds of interest include, for example, chemotherapeutic agents, hormones or hormone antagonists, growth factors or recombinant growth factors and fragments and variants thereof. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Water, Salts and Ions; Drugs Affecting Renal Function and Electrolyte Metabolism; Drugs Affecting Gastrointestinal Function; Chemotherapy of Microbial Diseases; Chemotherapy of Neoplastic Diseases; Drugs Acting on Blood-Forming organs; Hormones and Hormone Antagonists; Vitamins, Dermatology; and Toxicology, all incorporated herein by reference. Also included are toxins, and biological and chemical warfare agents, for example see Somani, S. M. (Ed.), “Chemical Warfare Agents,” Academic Press, New York, 1992).

Compounds, including candidate agents, can be obtained from a variety of sources including libraries of synthetic or natural compounds. Various means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, etc.; biological samples, e.g. lysates prepared from crops, tissue samples, etc.; manufacturing samples, e.g. time course during preparation of pharmaceuticals; as well as libraries of compounds prepared for analysis; and the like.

In some embodiments, the effect of the agent is determined by quantifiable parameters of stem cell-derived cardiomyocytes can include contractile force, contractility, altered contraction, frequency of contraction, contraction duration, contraction stamina, cardiomyocyte size, sarcomere organization, length, structure, metabolic respiratory capacity, oxygen consumption, and electrophysiological and biophysical parameters. In some embodiments, quantifiable parameters include differentiation, survival and regeneration of the stem cell-derived cardiomyocytes.

A plurality of assays comprising stem cell-derived cardiomyocytes can be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Optionally, the stem cell-derived cardiomyocytes used in the screen can be manipulated to express desired gene products.

Kits

In some embodiments, the compositions as described herein can be prepared as a kit. In one embodiment, a kit can comprise a nanopatterned substrate as described herein, thyroid hormone T3 or an analogue thereof, a vector encoding a Let7i microRNA or a preparation comprising a Let7i miRNA, and packaging materials therefor. In another embodiment, the kit further comprises an iPS cell or ES cell preparation, which can be metabolically active or frozen, and can optionally include reagents as described herein for differentiating cells of the iPS cell or ES cell preparation to a cardiomyocyte phenotype. In another embodiment, the stem cell-derived cardiomyocytes are pre-plated on or attached to the nanopatterned substrate.

In other embodiments, a kit further comprises cell culture medium and instructions to permit preparation of mature in vitro differentiated cardiomyocytes from the stem cell-derived cardiomyocytes. In one embodiment, the kit comprises a fatty acid preparation suitable for addition to tissue culture medium. Fatty acid preparations can include, for example, preparations comprising palmitate, oleic acid, linoleic acid, or combinations thereof.

In another embodiment, the kit comprises stem cell-derived cardiomyocytes, which can be metabolically active or frozen. In another embodiment, the kit and/or any of its constituents can be shipped and/or stored at ambient or room temperature, or at, e.g., 4° C.

In some embodiments, the iPS cells, ES cells, or stem cell-derived cardiomyocytes are human cells, rodent cells, canine cells, and the like. In some embodiments, the stem cell-derived cardiomyocytes are derived from a subject with a muscular disease or disorder or are genetically modified to mimic a muscular disease or disorder, including, for example, a cardiac disease or disorder.

### EXAMPLES

### Example 1: Engineered Developmental Niche Enables Predictive Phenotypic Screening in Human Dystrophic Cardiomyopathy

Cardiovascular disease remains the leading cause of death for both men and women worldwide, with a rapidly growing impact on developing nations¹. Inherited cardiomyopathies are the major cause of heart disease in all age groups, including children and young, otherwise healthy adults². Human pluripotent stem cell-derived cardiomyocytes (hPSC-CMs) are a promising tool for studying cardiomyopathy because they vastly increase the accessibility to human cardiomyocytes that can theoretically express the full array of ion channels, protein isoforms, and genetic and metabolic machinery found in the human heart³. A major goal of current cardiac disease modeling efforts is to gain insight into the onset and progression of cardiomyopathies as a first step towards designing more effective therapeutic strategies. Many patients with inherited cardiomyopathies, however, do not present with cardiac symptoms until late adolescence or early adulthood². This limits the potential of hPSC-CM disease modeling since current differentiation protocols produce cardiomyocytes with structural, functional, and biochemical properties that most closely resemble cells of the fetal heart⁴.

Developmental maturation presents a technical hurdle for hPSC-CMs in cardiac disease models. To date, hPSC-CMs exhibiting a fetal-like phenotype have been used to study channelopathies⁵⁻⁷, metabolic syndromes⁸, hypertrophic and dilated cardiomyopathies^{9,10}, as well as other delayed-onset cardiac diseases such as Duchenne Muscular Dystrophy (DMD) cardiomyopathyl^{1,12}. While such reports have certainly been groundbreaking and valuable¹³, the majority of the findings are based on the performance of cardiomyocytes that are still quite naive, having incomplete structural organization, and a mixture of fetal and adult protein isoform expression. Much effort has been invested to develop methods to accelerate the development of hPSC-CMs^{4,13}. While such strategies have proven to be effective at enhancing specific aspects of hPSC-CM development, it is unlikely that a single method can fully recreate the developing cardiac microenvironmental niche. A multifaceted and combinatorial maturation (ComboMat) platform was generated, incorporating nanotopographic substrate cues, thyroid hormone T3, and Let-7i miRNA overexpression aimed at more comprehensively mimicking the developmental cardiac niche in order to accelerate the maturation of hPSC-CMs in vitro. Anisotropic extracellular matrix cues, critical for proper structure-function relationship in the fully developed heart and morphological development in hPSC-CMs¹⁴, have also been found to be an important factor during cardiac development and looping¹⁵. Thyroid hormone treatment, simulating the spike in serum T3 levels immediately following birth, has been shown to improve force production and calcium handling in hPSC-CMs¹⁶, while upregulation of Let-7 miRNAs enhance metabolic development in hPSC-CMs¹⁷.

As a means to highlight the utility of a mature cardiac phenotype for in vitro cardiac tissue engineering applications, a more mature model of dystrophic cardiomyopathy was developed. Dystrophic cardiomyopathies, such as Duchenne muscular dystrophy (DMD) and Becker muscular dystrophy (BMD), are X-linked genetic disorders resulting from a mutated dystrophin gene. Cardiac complications have replaced respiratory failure as the leading cause of death in DMD patients due to the emergence of supportive ventilation as a standard of care¹⁸. However, cardiomyopathy in dystrophic rodent models, such as the mdx mouse, have proven to be poor predictors of patients' responses to therapeutic treatment in the clinic^{19,20}. In fact, a recent clinical trial with sildenafil, which showed promise in alleviating cardiac dysfunction in the mdx mouse²¹, exacerbated cardiac symptoms in DMD patients and had to be terminated early²⁰. Thus, an unmet medical need exists for improved human models of dystrophic cardiomyopathy. It has been previously reported that dystrophin mutant human cardiomyocytes displayed phenotypic differences compared to healthy controls, but these differences were mild or were induced by exogenous acute stress, such as hypotonic challenge^{11,22.} Other dystrophin mutant models that, developed by Lin et al.¹² and Young et al.²³, provide valuable insight into the DMD-specific disease mechanisms and potential therapeutic treatment options, but both reports were based on fetal-like hPSC-CMs. To not be bound by a particular theory, it was hypothesized that more mature hPSC-CMs will improve the predictive model of dystrophic cardiomyopathy found in patients.

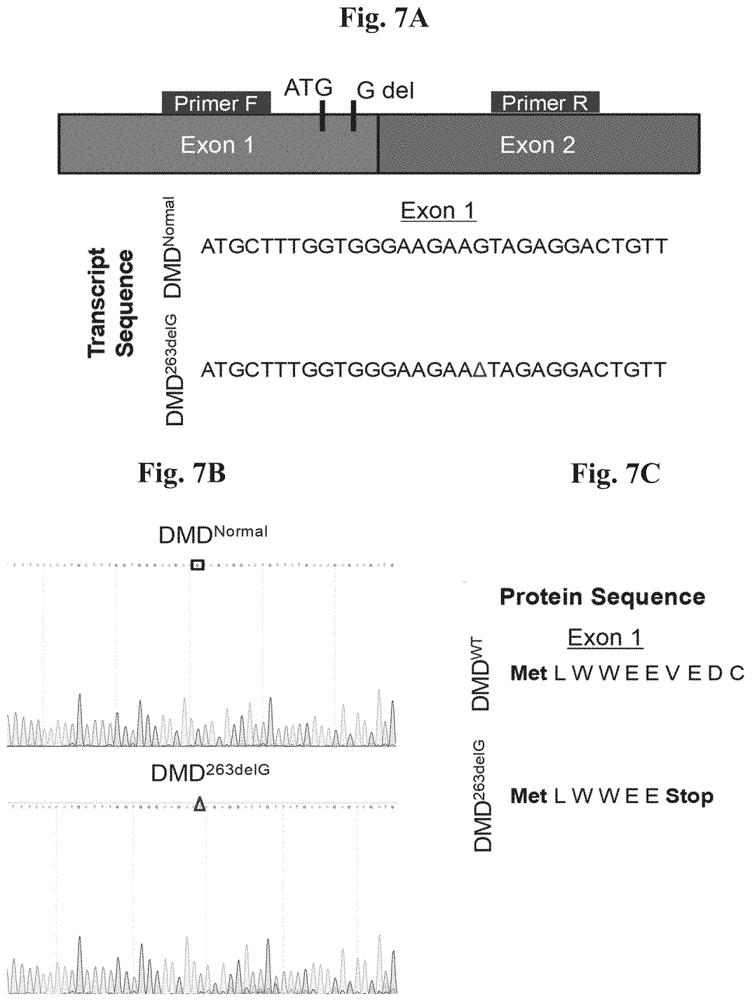

To test this hypothesis, the ComboMat platform was applied to CRISPR-generated dystrophin mutant (DMD 263delG) hPSC-CMs in order to facilitate the manifestation of adult-onset cardiac disease phenotypes in vitro. Patients with similar exon 1 mutations in the DMD gene (FIG. 7) express a truncated dystrophin protein and present with a BMD phenotype²⁴. When matured using a combinatorial, DMD mutant hPSC-CMs exhibit a much greater propensity for arrhythmia on a custom nanopattemed multielectrode array (nanoMEA) cardiac screening platform. Without developmental cardiac niche cues, it was not possible to distinguish the functional profile of DMD mutant hPSC-CMs from healthy isogenic control cells. Thus, the ComboMat platform produced more physiologically relevant hPSC-CMs for disease modeling and drug screening applications.

Results: Profiling the Transcriptome of ComboMat-Treated Cardiomyocytes

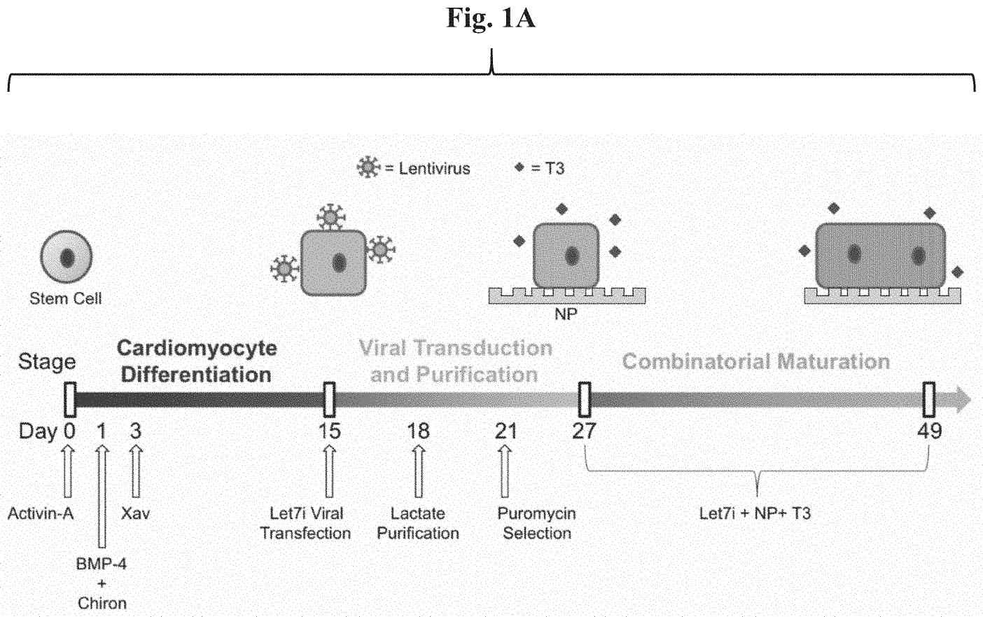

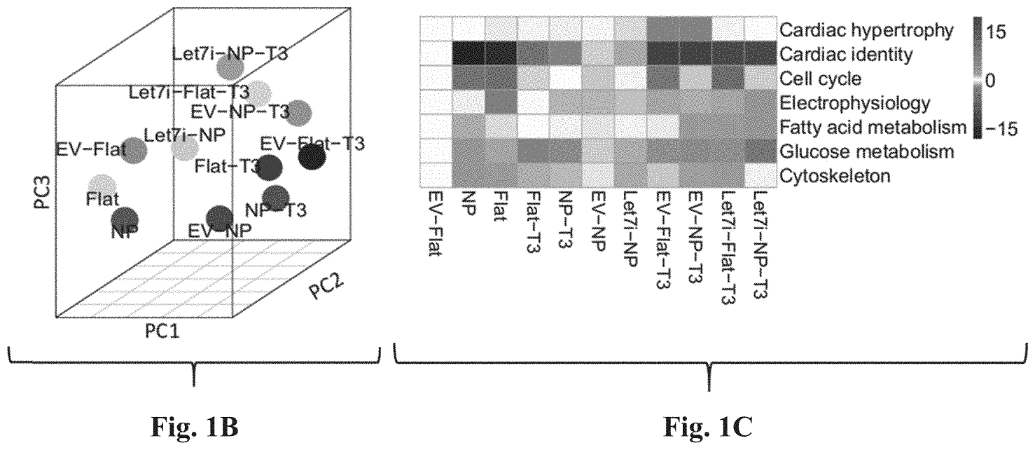

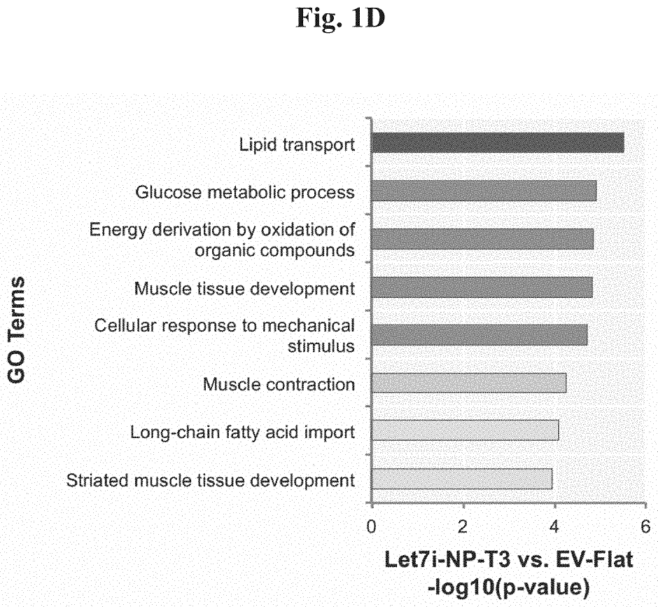

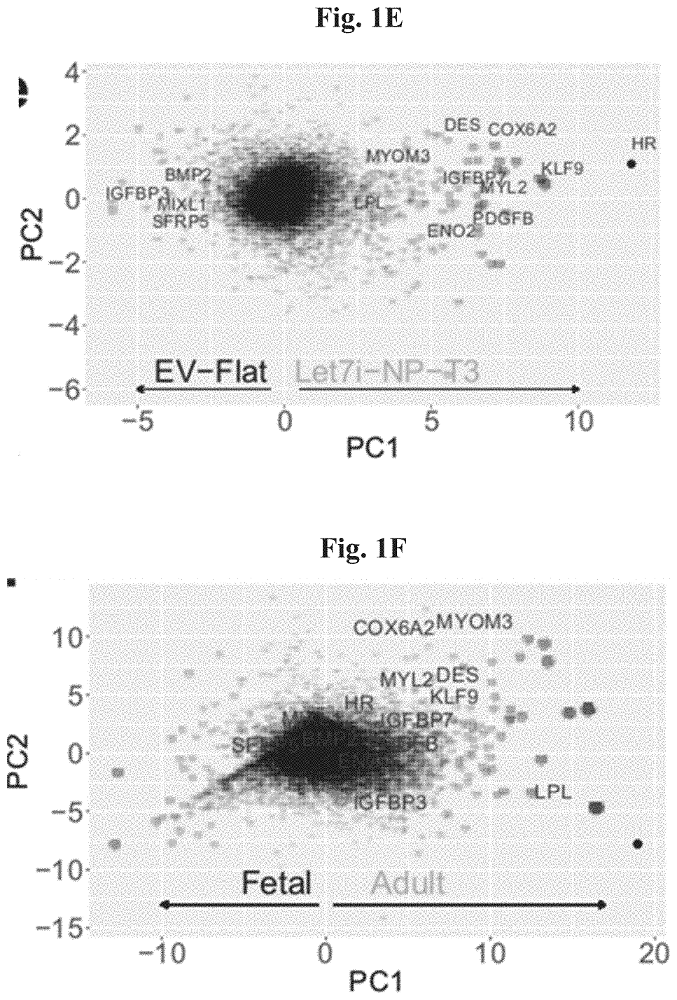

The combinatorial impact of three distinct cues were investigated which include biomimetic, nanopatterned topography (NP)^{14,15}, thyroid hormone T3 (T3)¹⁶, and Let7i miRNA overexpression (Let7i)¹⁷, incorporated into a single procedure termed the ComboMat platform (schematically depicted in FIG. 1A). RNA-seq was performed to look at the whole transcriptome of hPSC-CMs exposed to different combinations of the three maturation cues. Principal component analysis (FIG. 1B) demonstrated that the ComboMat group (all three factors combined together) separated from all other conditions. Next, an enrichment heat map was generated that consists of seven hallmark pathways of cardiomyocyte maturation, (FIG. 1C). As more maturation conditions were layered on top of one another, cardiomyocytes appeared to be progressively more mature. The ComboMat group showed the most robust maturation, with up-regulation in six of the pathways and down-regulation in the cell cycle pathway. FIG. 1D shows 8 up-regulated gene ontology (GO) terms based on P-value between the ComboMat and Control (Empty Vector cells on flat surfaces; EV-Flat) groups. Pathways related to metabolism such as glucose metabolic processes, long-chain fatty acid import, and glycolytic process were up-regulated. GO terms associated with muscle processes including muscle tissue development, muscle contraction, and striated muscle cell differentiation were also up-regulated.

A bubble plot was generated (FIG. 1E) to compare gene expression of the ComboMat and Control groups along PC1. Fifty-three (53) genes were discovered that were significantly higher and 13 genes that were significantly lower in both Adult vs. Fetal and ComboMat vs. Control. The gene that was most up-regulated along PC1 in the ComboMat group was Hair Growth Associated gene (HR), a thyroid hormone co-repressor. Krupel-like factor (KLF9), a transcription factor, was also significantly up-regulated. KLF9 has been reported to bind to the PPAR-gamma (γ) promoter, a critical gene in fatty acid metabolism²⁵. Cytochrome c oxidase subunit 6A2 (COX6A2), another gene associated with cardiac metabolism, was also up-regulated in the ComboMat group. Furthermore, myosin light chain 2 (MYL2), the ventricular isoform and a hallmark of cardiomyocyte maturation, was up-regulated. A bubble plot displays human fetal and adult cardiomyocyte gene expression (FIG. 1F) compared to the same genes highlighted in FIG. 1E. The highly up-regulated genes in the ComboMat group are also up-regulated in adult cardiomyocytes when compared to fetal controls.

Structural Maturation of ComboMat-Treated Cardiomyocytes

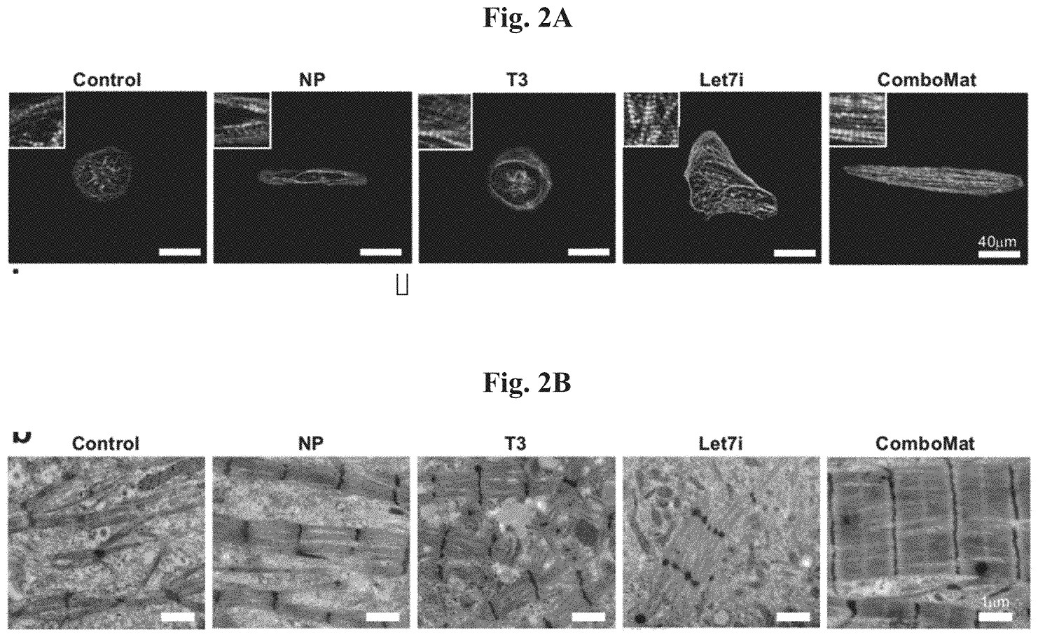

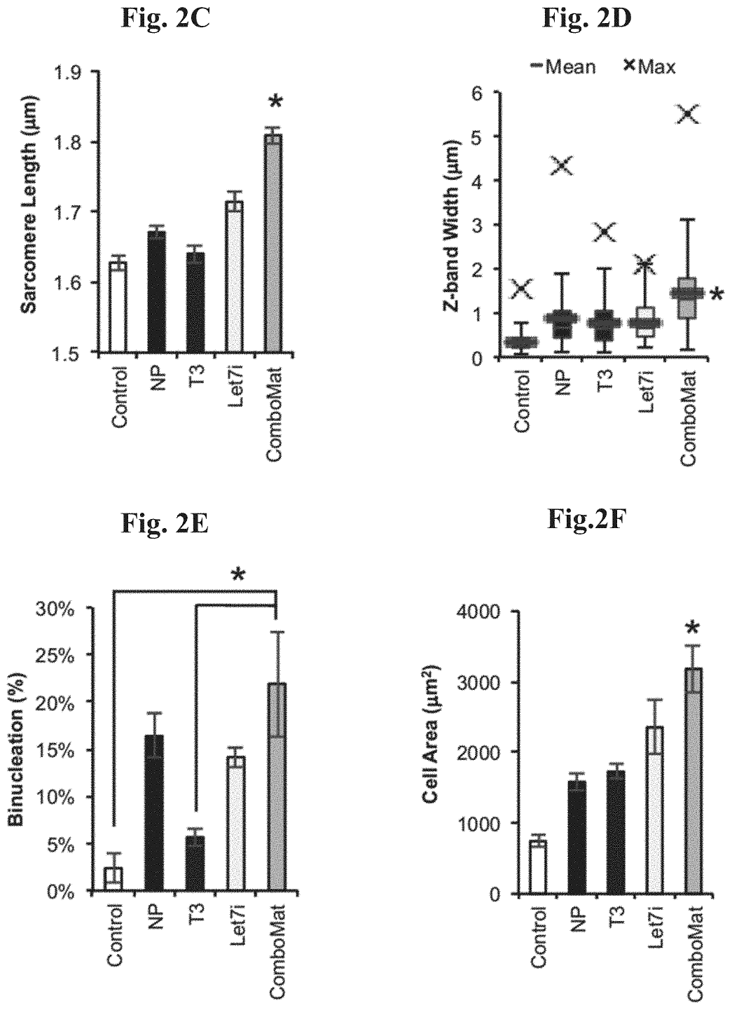

For these studies, and all subsequent functional assays, the full ComboMat platform (Let7i+NP+T3) was compared to a viral vector Control (EV+Flat+No T3) and each maturation cue in isolation: NP (EV+NP+No T3), T3 (EV+Flat+T3), and Let7i (Let7i+Flat+No T3). Immunofluorescence imaging (FIG. 2A) confirmed that hPSC-CMs cultured on NPs elongated in the direction of the nanotopography and exhibited more anisotropic, rod-shaped morphologies. Sarcomeres developed with a higher degree of directionality and order on NPs, resulting in more sarcomeres in register with one another (FIG. 2B). Compared to Control, hPSC-CMs exposed to just T3 or Let7i were significantly larger (FIG. 2F) but formed rounded or irregular morphologies. When exposed to all three cues in the ComboMat platform, hPSC-CMs became rod-shaped and were larger than with each cue in isolation (FIG. 2F, *p<0.05). hPSC-CMs exposed to the ComboMat platform also displayed a repetitive sarcomere banding pattern along the length of the cell in contrast to the circumferential banding found in the Control group, and had longer resting sarcomere lengths of approximately 1.8 micrometers (μm) ±0.012 micrometers (μm) (FIG. 2C, *p<0.05). hPSC-CMs in the ComboMat group also exhibited a higher binucleation percentage (FIG. 2E, *p<0.05).

The ultrastructure of the hPSC-CMs was investigated via transmission electron microscopy (TEM). It was observed that hPSC-CMs exposed to Control conditions exhibited low density, disorganized myofibrils and only punctate Z-body formation (FIG. 2B). The application of NPs, T3, or Let7i individually improved the development of more organized and wider Z-bands (FIG. 2D) but overall sarcomeres remained rather disorganized and at a low density. In contrast, hPSC-CMs cultured with the ComboMat platform developed much more ordered sarcomeres, with the emergence of Z-bands and H-zones (FIG. 2B). Z-band width in cells, an indicator of myofibril bundling, was also significantly larger in the ComboMat group than Control or each cue in isolation.

Electro-Mechanical and Metabolic Maturation of ComboMat-Treated Cardiomyocytes

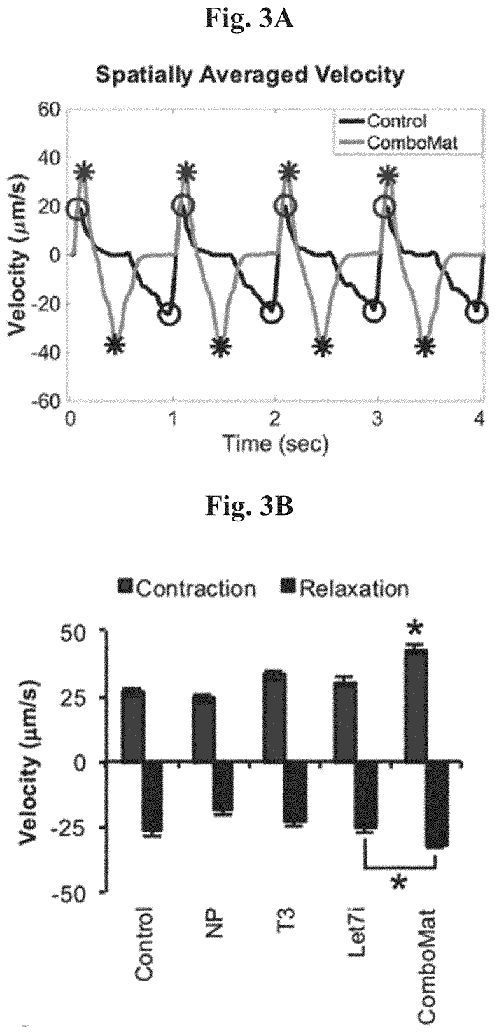

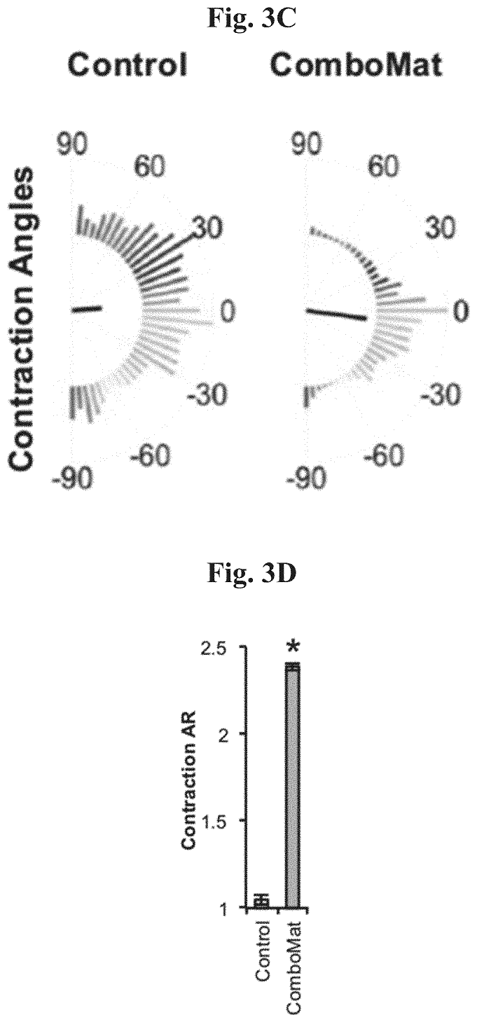

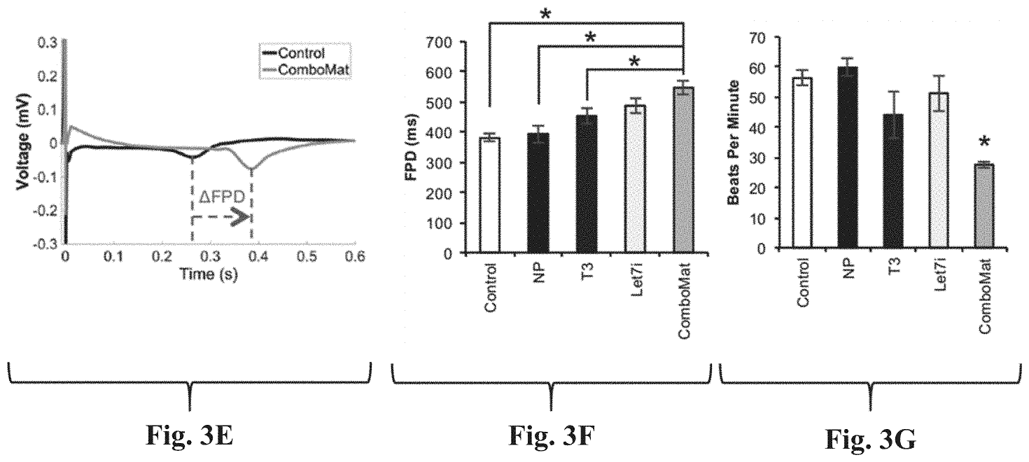

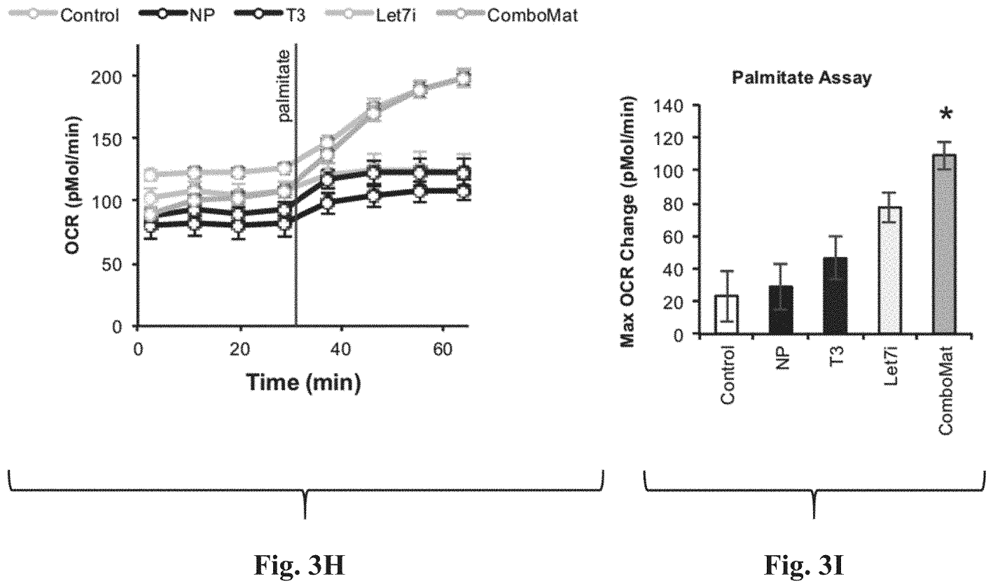

To test if the genetic and structural changes imparted by the ComboMat platform manifested into enhanced functional performance of the hPSC-CMs, two noninvasive measurements were employed to determine electromechanical function. Using correlation-based contraction quantification (CCQ)²², contractions of hPSC-CM monolayers were measured and paced at 1 Hz and filmed at 30 frames per second. By spatially averaging these contraction vectors together, maximum contraction and relaxation velocities were measured (annotated as red and blue marks in FIG. 3A) for the Control, NP, T3, Let7i, and ComboMat conditions and found that cells exposed to the ComboMat platform had significantly faster contractions compared to Control or any maturation cue in isolation (FIG. 3B). To help explain this, the angles of displacement during contractions were examined and it was discovered that hPSC-CMs cultured on NPs contract in a more uniform direction than cells cultured on flat substrates (FIG. 3C). To quantify this preferred directionality, the contraction anisotropic ratio (AR) was measured and it was determined that cells on NPs, such as those in the ComboMat platform, had a contraction magnitude over two times greater in the direction parallel with the underlying nanotopography than perpendicular to it (ComboMat AR=2.39±0.01) (FIG. 3D). This was in contrast to cells grown on traditional flat substrates, where contractions were randomly oriented, and exhibited similar magnitudes in all directions (Control AR=1.04±0.02).

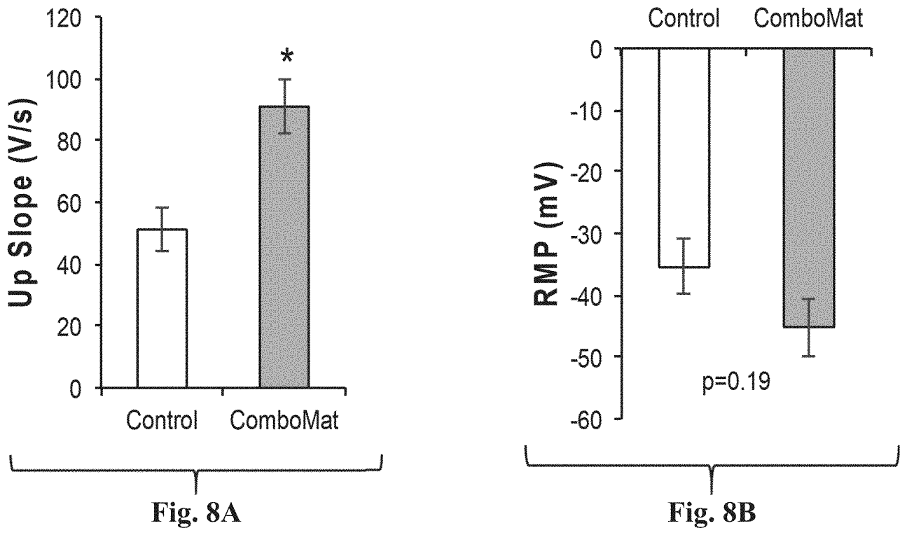

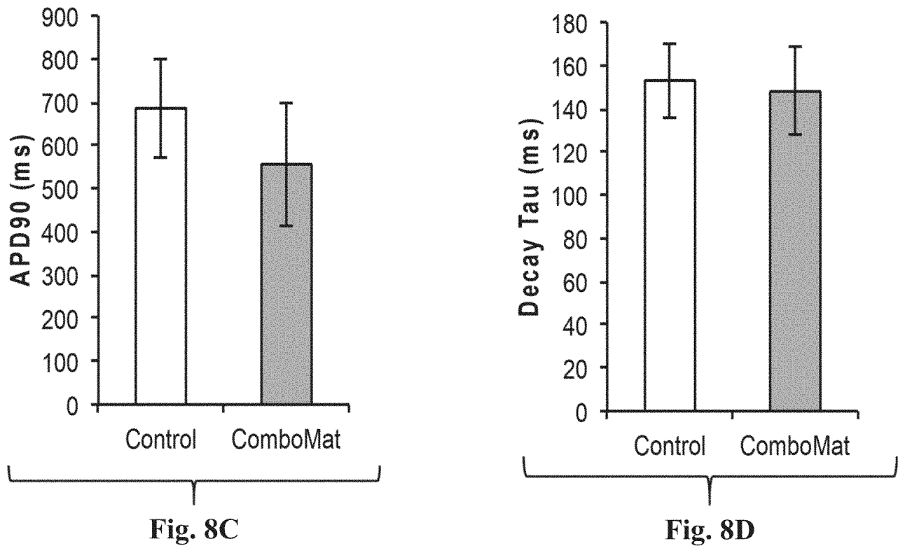

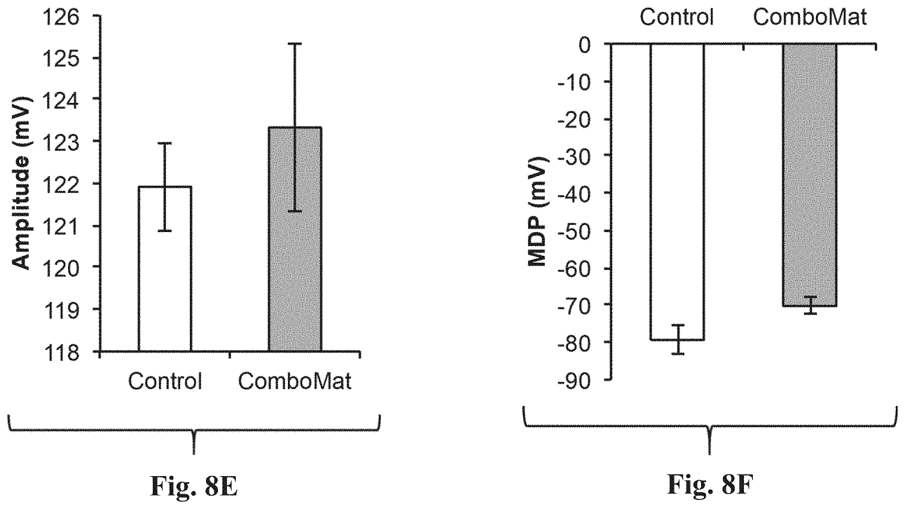

In addition to contraction dynamics, cardiomyocyte electrical activity was measured using microelectrode arrays (MEAs). An ion permeable resin, Nafion, was used to generate nanotopographic surfaces on MEAs (nanoMEAs) in order to facilitate hPSC-CM alignment in NP and ComboMat conditions while still enabling high resolution electrophysiological data capture from underlying electrodes. FIG. 3E shows temporally averaged field potential recordings from Control and ComboMat cultures. Measuring spontaneous electrical activity of cardiac monolayers, it was discovered that hPSC-CMs exposed to the ComboMat platform had significantly longer field potential durations than the Control, NP, and T3 groups (FIG. 3F). Additionally, the ComboMat platform significantly slowed the beat rate of the cardiomyocytes compared to Control or each maturation cue in isolation (FIG. 3G). hPSC-CMs exposed to the ComboMat platform also exhibited faster upstroke velocity as measured via patch clamp (FIG. 8)

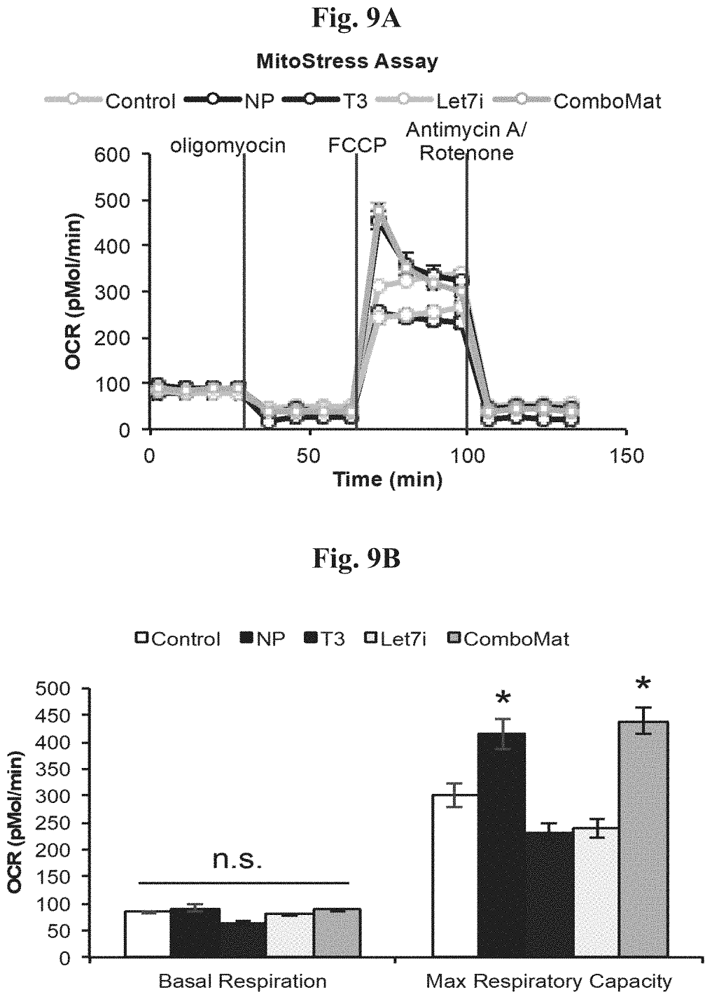

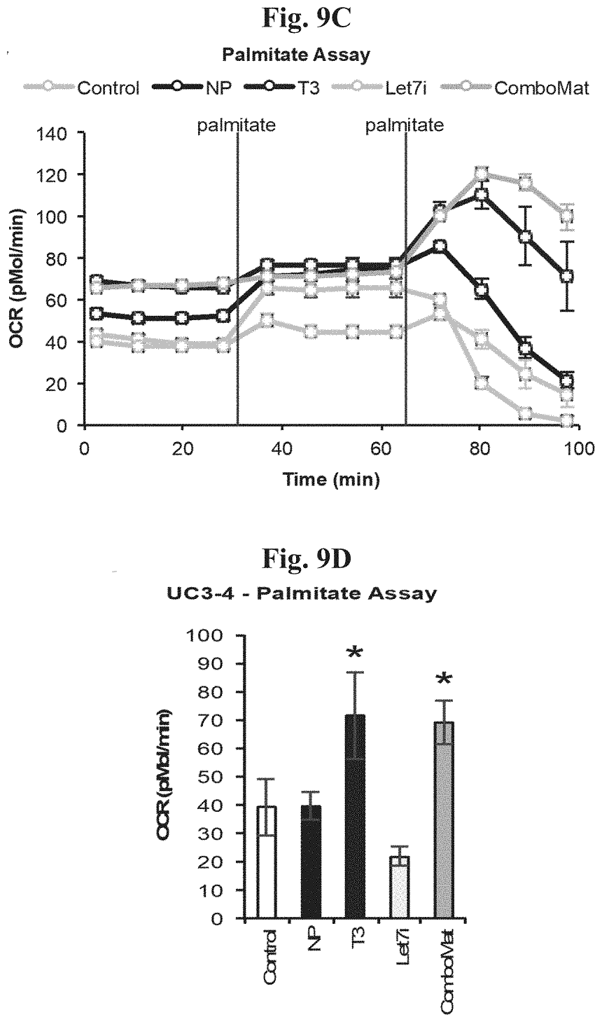

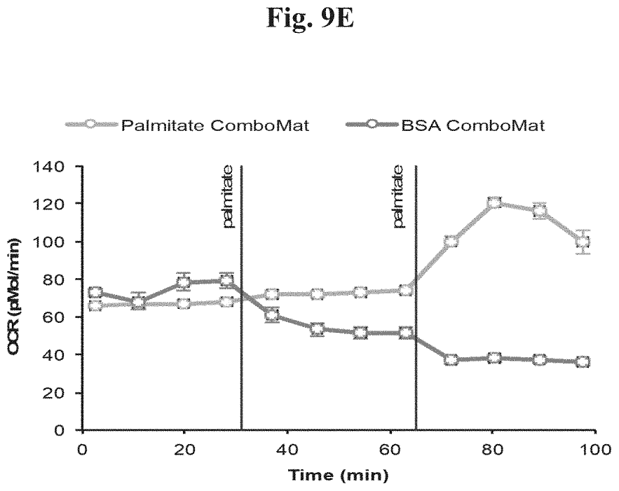

RNA-seq analysis suggested significant changes to cardiac metabolism imparted by the ComboMat platform. Thus, the ability of the cardiomyocytes to utilize exogenous fatty acids was probed via the “Palmitate assay” on a Seahorse mitochondrial flux analyzer (FIG. 3H). It was observed that hPSC-CMs exposed to the ComboMat platform exhibited significantly greater maximum change in oxygen consumption rate (OCR) measured between palmitate concentrations of 200 μM and 400 04. (FIG. 3I). Maximum respiratory capacity was also significantly greater in the ComboMat group compared to Control (FIG. 9)

ComboMat-Treated Dystrophin-Mutant hPSC-CMs Manifest Disease Phenotype

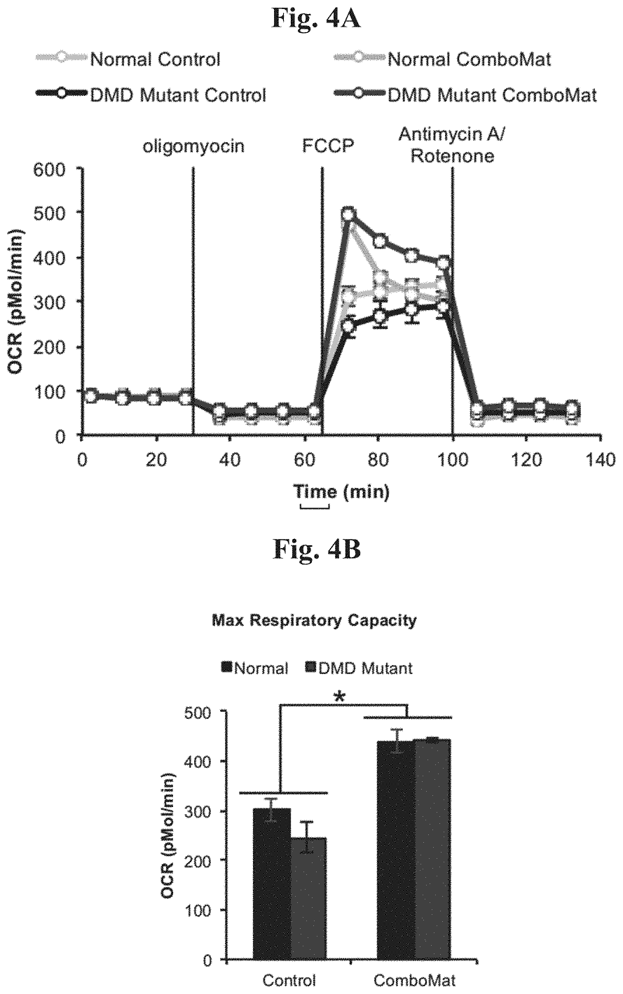

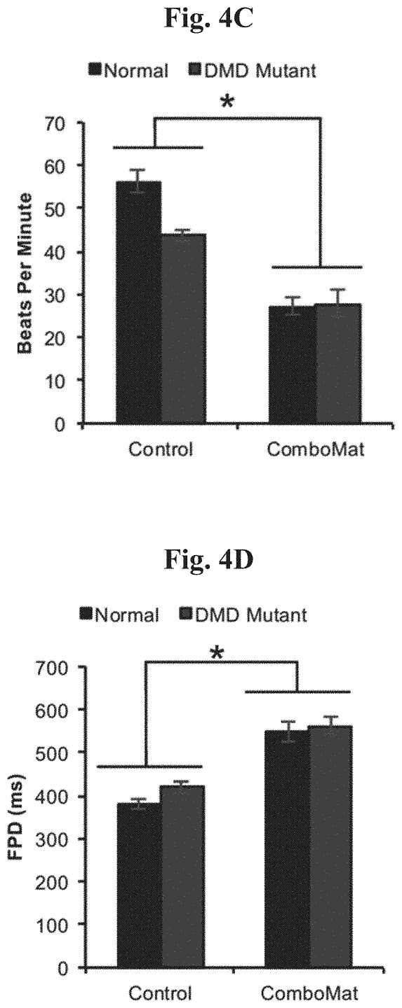

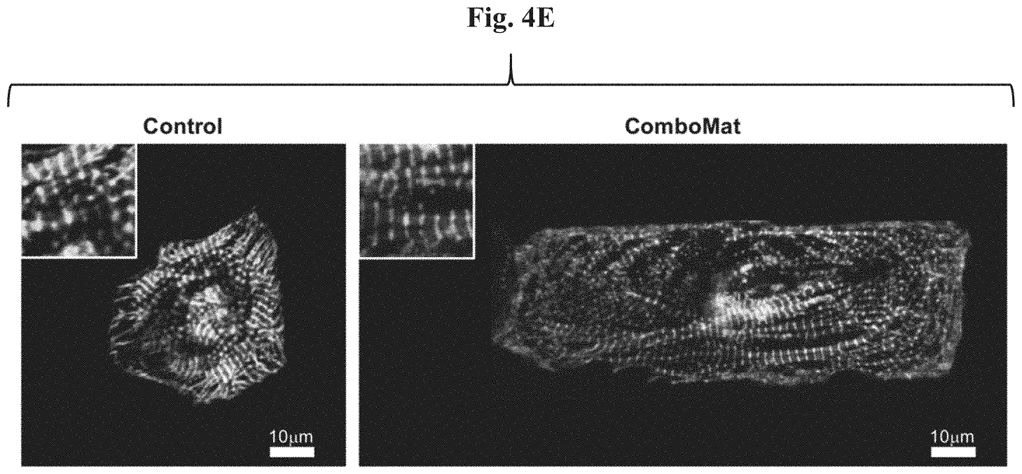

To begin, it was tested whether DMD mutant hPSC-CMs responded in a similar manner to the ComboMat platform as their Normal (UC3-4) isogenic counterparts. Using the Seahorse MitoStress assay (FIG. 4A), it was observed that the DMD mutant hiPSC-CMs had an increased maximum respiratory capacity when cultured under the ComboMat platform compared to Control conditions (FIG. 4B). Within the Control or ComboMat conditions, however, there was no statistical difference between the Normal and DMD mutant groups. The baseline electrophysiological characteristics of the DMD mutant cells were measured using a custom nanoMEA platform. The beat rate of the DMD mutant hiPSC-CMs decreased in response to the ComboMat platform to the same extent as the Normal hPSC-CMs (FIG. 4C). Additionally, the field potential duration (FPD) of DMD mutant hPSC-CMs increased when exposed to ComboMat compared to Control, mirroring observations made from Normal hPSC-CMs (FIG. 4D). From a structural standpoint, the DMD mutant hiPSC-CMs developed a characteristic elongated morphology with in-register sarcomeres when exposed to the ComboMat platform, whereas DMD mutant hiPSC-CMs exposed to Control conditions were more rounded, with more disorganized sarcomeres (FIG. 4E).

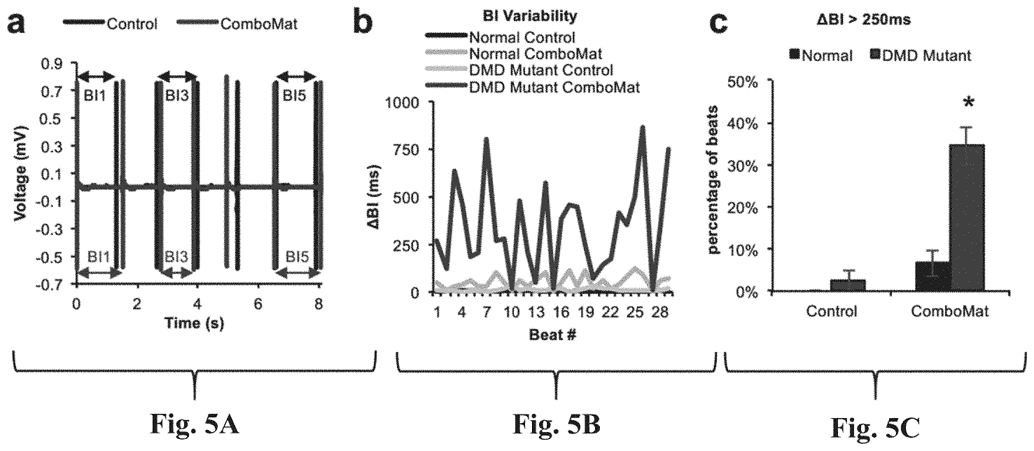

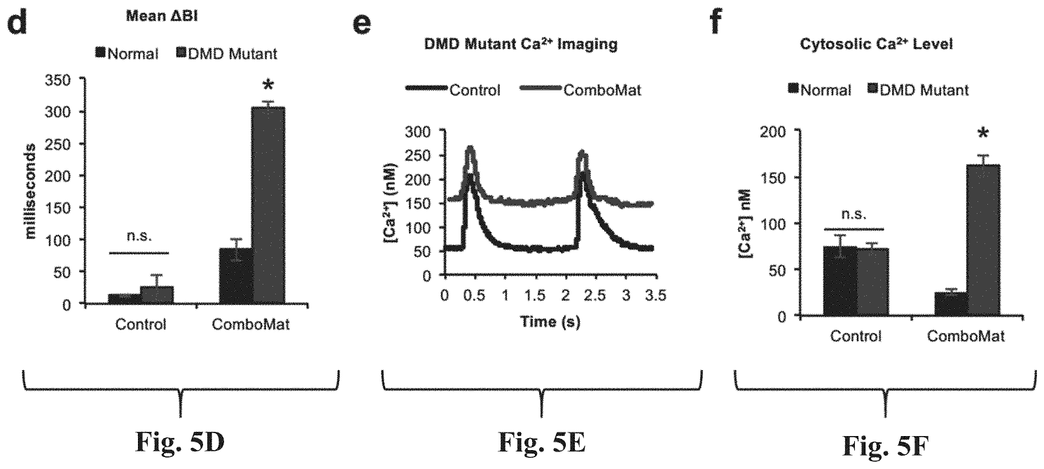

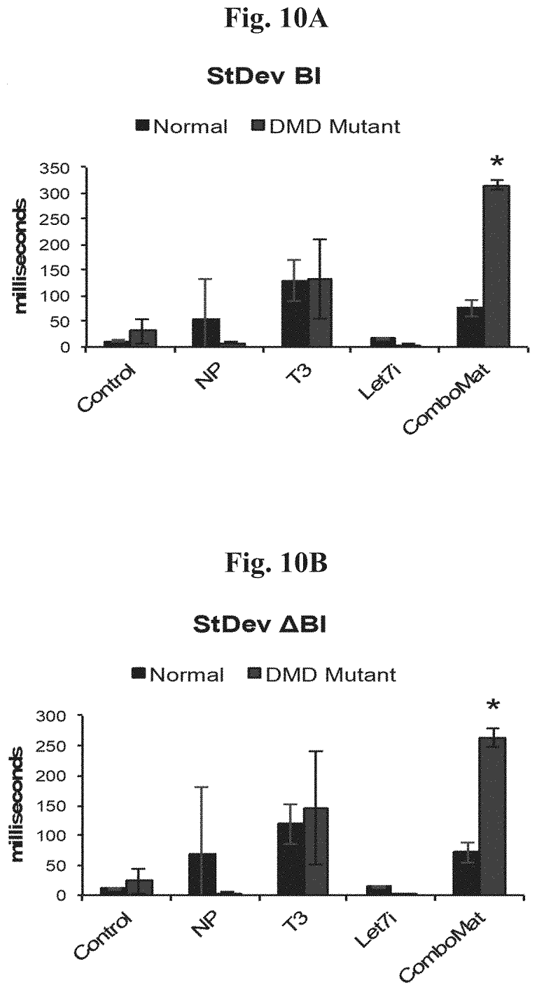

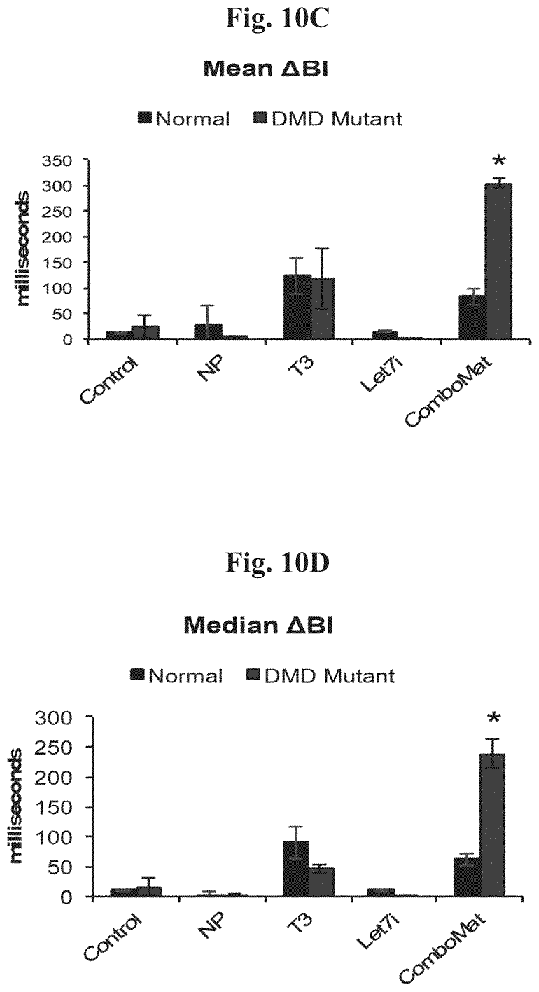

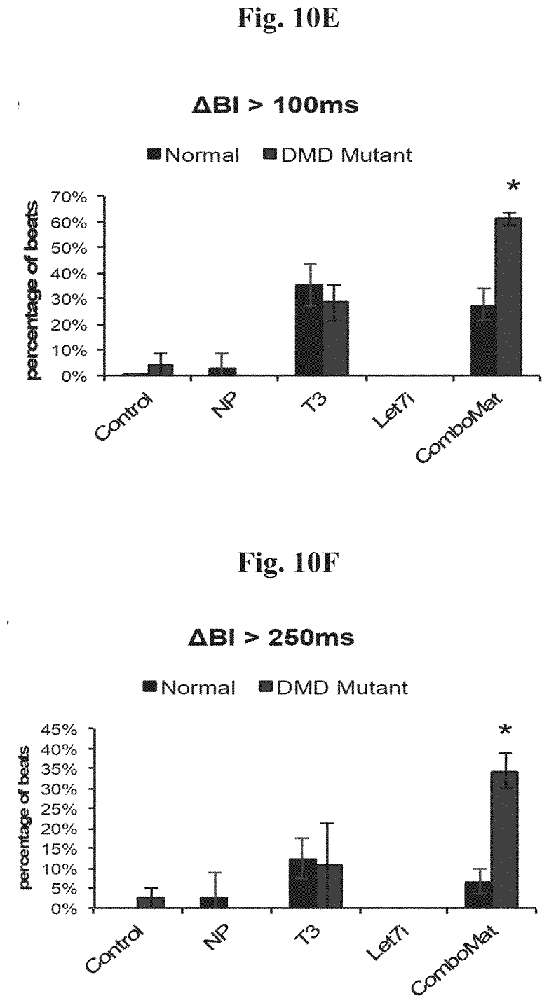

Without being bound by a particular theory, it was hypothesized that by inducing hypertrophy and overall cardiomyocyte maturation, dystrophic hPSC-CMs would undergo greater mechanical stress and cellular damage, resulting in irregular functional activity, which would be absent in immature cells. A nanoMEA platform was used to record the electrical activity of spontaneously beating monolayers of the isogenic pair of Normal and DMD mutant hPSC-CMs exposed to Control or ComboMat conditions. Using a method of arrhythmia detection relying on beat rate variability²⁶, it was observed that the DMD mutant hPSC-CMs exposed to the ComboMat platform were significantly more arrhythmogenic than less mature DMD mutant hPSC-CMs in the Control condition. FIG. 5A shows representative field potential traces from DMD mutant hPSC-CMs exposed to Control or ComboMat conditions with individual beat intervals annotated. By measuring the difference in time of the beat interval from one beat to the next (ΔBI) and plotting this change for 30 consecutive beats (FIG. 5B), it was discovered that matured DMD mutant hPSC-CMs had a striking instability in beat rate. By measuring the percentage of beats with a ΔBI>250 ms (FIG. 5C) and the arithmetic mean for ΔBI over 30 consecutive beats (FIG. 5D), it was observed that relatively large changes in beat intervals indicative of a more arrhythmogenic state. Without an autonomous nervous system in vitro, hPSC-CMs should develop a steady beating pattern without much variation from beat to beat. This steady behavior is apparent in the Control conditions for both normal and DMD cardiomyocytes as well as in healthy mature Normal cells (FIG. 5B). Importantly, it was observed that the full ComboMat platform is necessary to observe functional differences between Normal and DMD mutant hPSC-CMs. When the maturation cues are applied in isolation, there is no separation between the healthy Normal cells and the DMD mutant hPSC-CMs (FIG. 10).

To identify a potential mechanism for this beat rate variability, intracellular calcium (Ca²⁺) was measured using the ratiometric Ca²⁺ indicator Fura-2 on an IonOptix imaging setup calibrated to calculate nanomolar intracellular Ca²⁺ concentrations. FIG. 5E depicts representative Ca′ traces from DMD mutant hPSC-CMs exposed to Control or ComboMat conditions. Matured DMD mutant hPSC-CMs were found to have an elevated baseline, or diastolic resting Ca²⁺ level, compared to the less mature cells in the Control group. Quantifying this diastolic Ca²⁺ level, a similar trend to the electrical instability was observed as presented previously. Normal and DMD mutant hPSC-CMs in the Control groups had cytosolic Ca²⁺ levels that were not statistically different from one another (FIG. 5F). DMD mutant hPSC-CMs matured using the ComboMat platform, however, have an elevated cytosolic Ca²⁺ concentration compared to Normal hPSC-CMs.

Phenotypic Drug Screen Using Mature hPSC-CMs

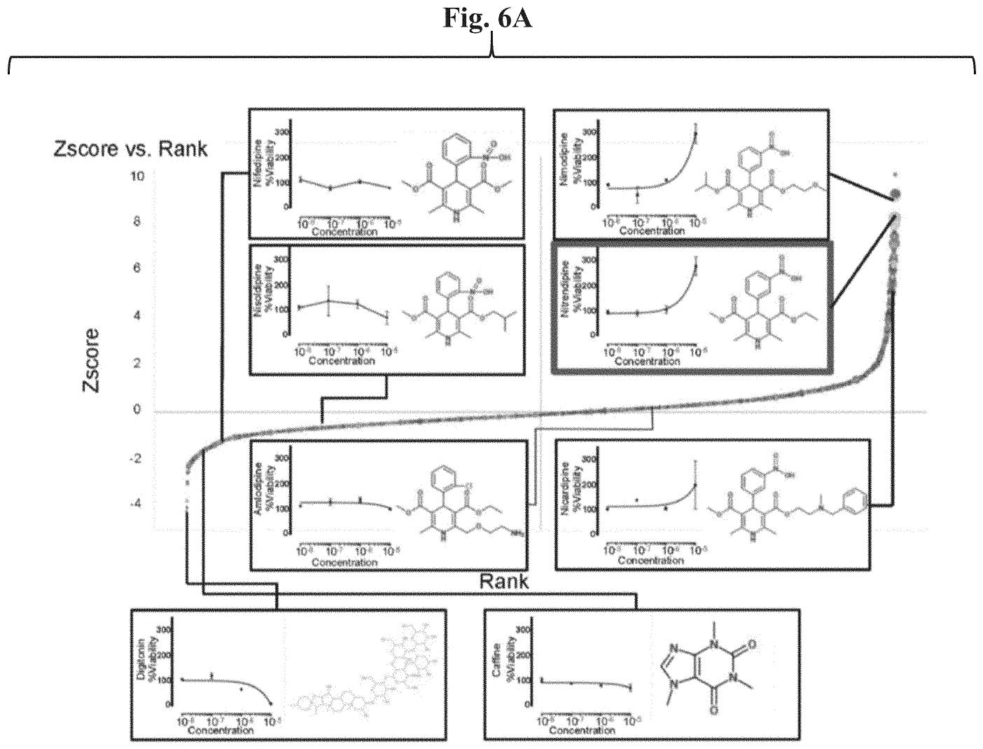

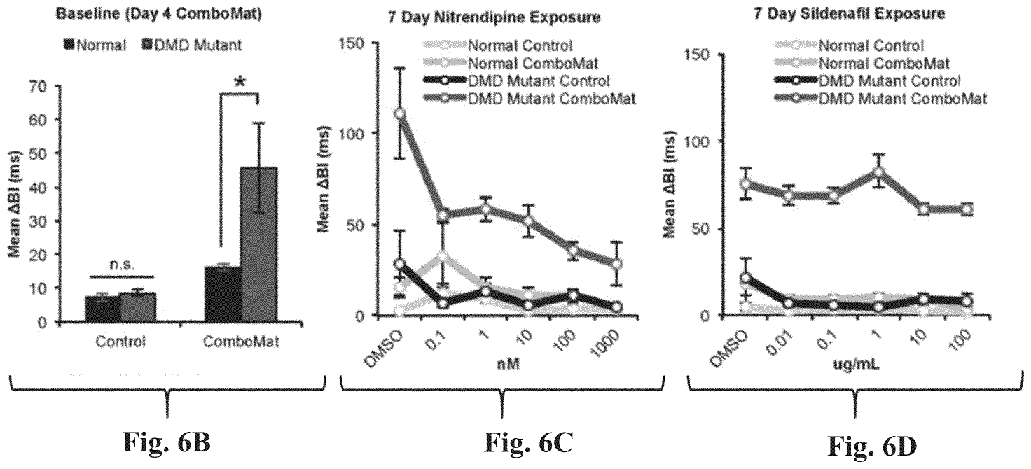

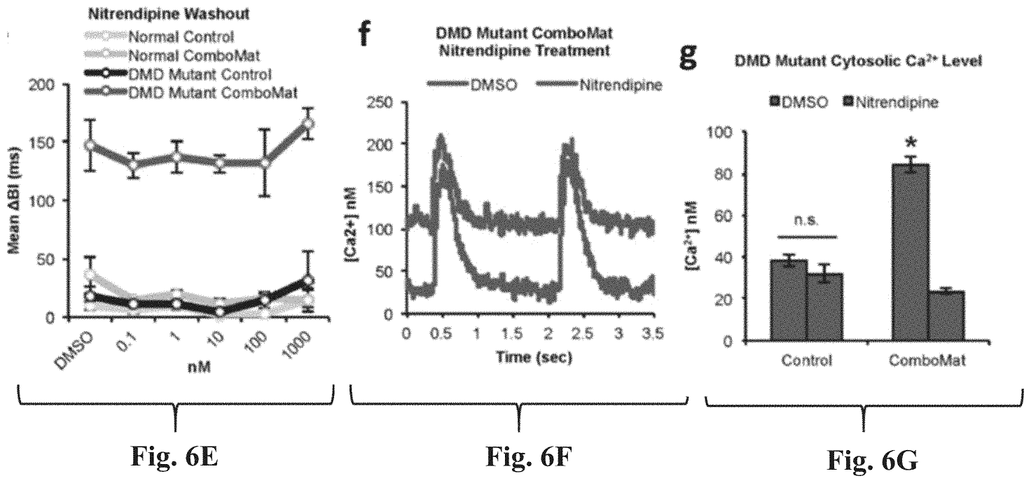

In order to identify potential drug targets or classes of drugs for a more detailed phenotypic drug screen, a preliminary medium throughput, semi-automated screen was performed with a small library of 2,000 diverse molecules with various mechanisms of action. Intracellular ATP (a widely used assay of cell viability in high throughput screens) was measured in DMD mutant hPSC-CMs after hypotonic stress. Overall the Z-prime of the assay was 0.71. The 8,000 data points (2,000 compounds at 4 concentrations) were ranked and plotted based on the Z-score (FIG. 6A). Taking into consideration the Z-score ranking (>3), standard deviation of replicates, and escalating dose-ranging response, 39 hits were identified from the 2000 input compounds (˜2% hit rate) (Table 2). Of these hits, 9 (˜23%) were classified as Ca²⁺ channel blockers.

TABLE 2
Primary screening hits.

| | Average of % | | |
|---|---|---|---|
| Compound | Viability | Z score | Mechanism of action |
| ACEPROMAZINE MALEATE | 239.1 ± 38.37 | 6.57 | Sedative |
| ACYCLOVIR | 258.33 ± 14.16 | 7.44 | Antiviral |
| AKLOMIDE | 276.60 ± 12.37 | 8.26 | Antiprotozoal, Coccidiostat |
| ALTRETAMINE | 188.22 ± 2.87 | 4.27 | Antineoplastic |
| AMINOHIPPURIC ACID | 207.49 ± 7.09 | 5.14 | Renal function diagnosis |
| AMIODARONE | 168.14 ± 8.45 | 3.37 | Adrenergic agonist, coronary |
| HYDROCHLORIDE | | | vasodilator, Ca channel |
| | | | blocker |
| ANTIMYCIN A | 180.04 ± 1.89 | 3.90 | Antifungal, antiviral, interferes |
| | | | in cytochrome oxidation |
| ASCORBIC ACID | 181.48 ± 23.68 | 3.97 | Antiscorbutic, antiviral |
| ATENOLOL | 171.27 ± 1.77 | 3.51 | Beta adrenergic blocker |
| BENZALKONIUM CHLORIDE | 194.64 ± 23.10 | 4.56 | Anti-infective (topical) |
| BEPRIDIL HYDROCHLORIDE | 178.65 ± 3.48 | 3.84 | Antiarrhythmic |
| BIOTIN | 315.95 ± 51.89 | 10.04 | Vitamin B complex |
| CANTHARIDIN | 239.59 ± 14.65 | 6.59 | Blister agent (terpenoid) |
| CEFDINIR | 185.70 ± 45.76 | 4.16 | Antibacterial |
| CHLORCYCLIZINE | 238.84 ± 35.23 | 6.56 | H1-antihistamine |
| HYDROCHLORIDE | | | |
| CINCHOPHEN | 168.44 ± 44.63 | 3.38 | Analgesic, antipyretic, |
| | | | antiinflammatory |
| CLOPIDOGREL SULFATE | 194.84 ± 50.3 | 4.57 | Platelet aggregation inhibitor |
| CORTISONE ACETATE | 205.92 ± 12.32 | 5.07 | Glucocorticoid |
| COTININE | 188.79 ± 56.0 | 4.30 | Antidepressant |
| CROTAMITON | 178.25 ± 27.22 | 3.82 | Antipruritic, Scabicide |
| DACARBAZINE | 177.63 ± 45.35 | 3.79 | Antineoplastic |
| DOXORUBICIN | 207.98 ± 34.29 | 5.16 | Antineoplastic |
| ESTROPIPATE | 208.65 ± 30.77 | 5.19 | Estrogen |
| GITOXIGENIN DIACETATE | 177.20 ± 1.17 | 3.77 | Cardiac glycoside |
| HALOPERIDOL | 197.30 ± 19.03 | 4.68 | Antidyskinetic, antipsychotic |
| HYDROXYUREA | 187.65 ± 49.61 | 4.25 | Antineoplastic, inhibits |
| | | | ribonucleoside diphosphate |
| | | | reductase |
| MEBHYDROLIN | 172.81 ± 39.86 | 3.58 | H1 antihistamine |
| NAPHTHALENESULFONATE | | | |
| NAPROXOL | 168.76 ± 2.61 | 3.39 | Antinflammatory, analgesic, |
| | | | antipyretic |
| NICOTINYL ALCOHOL | 262.12 ± 57.89 | 7.61 | Vasodilator |
| TARTRATE | | | |
| NIMODIPINE | 292.38 ± 56.17 | 8.97 | Calcium channel blocker, |
| | | | vasodilator |
| NITRENDIPINE | 275.48 ± 52.16 | 8.21 | Calcium channel blocker, |
| | | | vasodilator |
| OXYPHENBUTAZONE | 176.96 ± 42.53 | 3.76 | Anti-inflammatory |
| PODOFILOX | 168.46 ± 28.98 | 3.38 | Antineoplastic. inhibits |
| | | | microtubule assembly, and |
| | | | human DNA topoisomerase II; |
| | | | antimitotic agent |
| PROGLUMIDE | 175.69 ± 50.35 | 3.71 | Anticholinergic |
| PUROMYCIN | 187.84 ± 39.68 | 4.25 | Antineoplastic, antiprotozoal |
| HYDROCHLORIDE | | | |
| RETINYL PALMITATE | 236.79 ± 46.63 | 6.46 | Provitamin, |
| | | | antixerophthalamic |
| SELAMECTIN | 172.91 ± 25.29 | 3.58 | Antiparasitic, antimite |
| SULFADOXINE | 195.32 ± 23.52 | 4.59 | Antibacterial |
| TERFENADINE | 211.28 ± 22.46 | 5.31 | H1 antihistamine, nonsedating, |
| TILORONE | 172.53 ± 15.79 | 3.56 | Antiviral |

Incorporating this preliminary drug screen data with the findings of elevated intracellular Ca²⁺ content (FIG. 5F), the nanoMEA platform was utilized to test whether nitrendipine (highlighted with blue box in FIG. 6A), a dihydropyridine Ca²⁺ channel blocker, could reduce the arrhythmogenic behavior of the mature DMD mutant hiPSC-CMs. Sildenafil, an agent previously demonstrated not to benefit DMD patients²⁰ was also tested. Even at an early time point (four day ComboMat exposure) there was a significant difference in baseline beat rate variability between mature Normal hPSC-CMs and DMD mutant hPSC-CMs, while immature cells were indistinguishable from each other (FIG. 6A).

Cells were exposed to various concentrations of nitrendipine or sildenafil citrate for seven days and measured their beat rate variability. FIG. 6C-D shows the dose-response curves for nitrendipine and sildenafil, respectively. Nitrendipine significantly reduced the Mean ΔBI in mature DMD mutant hPSC-CMs compared to DMSO carrier control. Doses up to 1 μM nitrendipine restored beat rate variability to levels within physiologically relevant ranges (<50 ms). Higher doses, however, ceased spontaneous beating. In contrast, sildenafil had no effect on beat rate variability, regardless of the dose. Neither nitrendipine nor sildenafil had a significant impact on beat rate variability for cells exposed to Control conditions or Normal hPSC-CMs exposed to the ComboMat platform. The benefits of nitrendipine were washed out within 48 hours if removed from the cell culture medium (FIG. 6E). Ca²⁺ imaging revealed that 100 nM nitrendipine reduced the diastolic Ca²⁺ content in the mature DMD mutant hPSC-CMs compared to DMSO control (FIG. 6F-G). Nitrendipine had no impact on the diastolic Ca²⁺ content in cells exposed to the Control condition (FIG. 6G).

### DISCUSSION

The ComboMat platform proved to be a powerful tool in modulating cardiomyocyte development in vitro, increasing cell area, producing longer resting sarcomere lengths, and achieving more physiologically relevant binucleation percentages. Many of the functional enhancements that were observed from the ComboMat platform were predicted by RNAseq analysis. GO terms for muscle development and contraction were upregulated in the ComboMat group, which resulted in faster contraction kinetics as measured by CCQ contraction mapping. Metabolic GO terms such as fatty acid import were also upregulated in the ComboMat group and subsequently it was observed that cells exposed to the ComboMat group had a larger maximum respiratory capacity and ability to utilize exogenous fatty acids. Thus, there was agreement between the transcriptional and functional changes imparted by the ComboMat platform providing validation of the methods provided herein.

An important aspect of the methods described herein is the development of a successful phenotypic drug screen platform. Methods such as the ComboMat platform, in conjunction with the nanoMEA electrophysiological screening system, can be scaled up for higher throughput screens of novel compounds. The noninvasive measurements can be used to monitor cell function longitudinally and the strong stratification of disease phenotype allows for easy comparison to healthy controls to see if drug treatment correlates with a restoration of function. Important Ca²⁺ channel blockers were validated in the screen and it was possible to exclude a false positive drug hit from mdx mouse model studies²⁰.

Previous hPSC-CM models of dystrophic cardiomyopathy have relied heavily on hypotonic challenge to bring out a measurable disease phenotype^{11,23.} While effective, hypotonic challenge is not physiologically accurate and is only a transient insult. Thus, by maturing the DMD mutant hPSC-CMs with the ComboMat platform, cells present with beat rate variability due in part to calcium overload. Along with its involvement in mediating cell injury through reactive oxygen species (ROS) and mitochondrial apoptosis signaling pathways²⁷, Ca²⁺ overload has long been known to mediate cardiac arrhythmias²⁸ and has been implicated both in vitro and in vivo to be a driving force behind DMD pathophysiology²⁹. Thus, a maturation-dependent disease phenotype in DMD mutant hPSC-CMs has been demonstrated that recreates an early disease phenotype found in patients. This is an important distinction from previous DMD disease models and one that represents a logical progression in the cardiac disease modeling field. Wen et al. also reported the need for maturation in their hPSC-CM model of arrhythmogenic right ventricular dysplasia in order to bring out a disease phenotype³⁰ and Tiburcy et al. demonstrated maturation via chronic catecholamine stimulation improved their ability to model heart failure³¹. Based on these findings, other cardiomyopathies can be faithfully modeled by using hPSC-CMs matured beyond the typical fetal stage of standard directed differentiation methods using the methods and compositions described herein.

While it is known that DMD patients exhibit diastolic dysfunction and arrhythmias, the pathogenesis of this disease phenotype is not fully understooe. Ca²⁺ overload has long been postulated to play a central role in the pathophysiology of dystrophic cardiomyopathy²⁹. Leaky Ca²⁺ channels on the cell membrane and sarcoplasmic reticulum, abnormal NO signaling, and tears in the sarcolemma have all been proposed as the means for Ca²⁺ entry into the cell but no consensus exists. The present model, with an endogenous electrophysiological disease phenotype due to elevated Ca²⁺ levels, prove to be a valuable tool for addressing some of these questions. Taken together, the present data show that cardiac maturation is an important factor in accurately modeling adult onset diseases, such as DMD cardiomyopathy, and that the ComboMat platform is essential in generating more mature hPSC-CMs. Furthermore, nanoMEA platform can be used in conjunction to effectively screen potential therapeutic targets.

### Example 2: Methods

### Experimental Groups

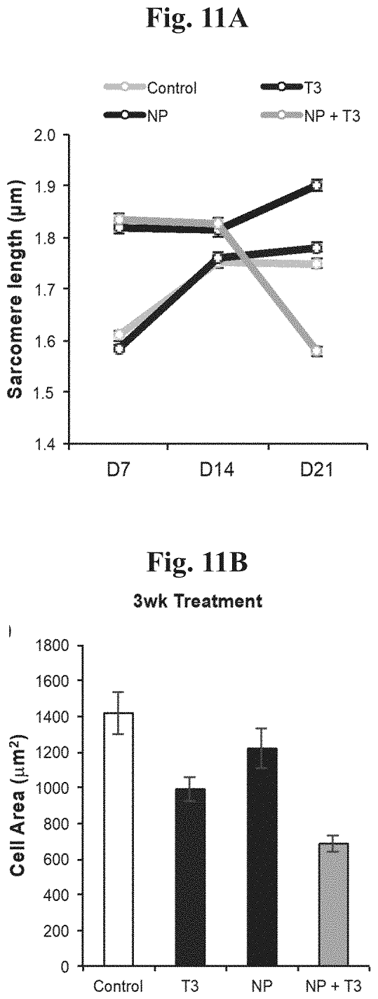

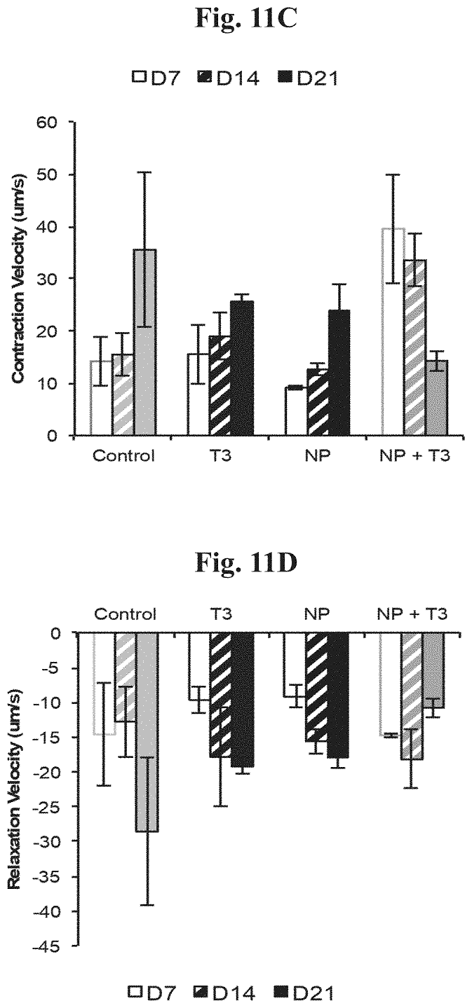

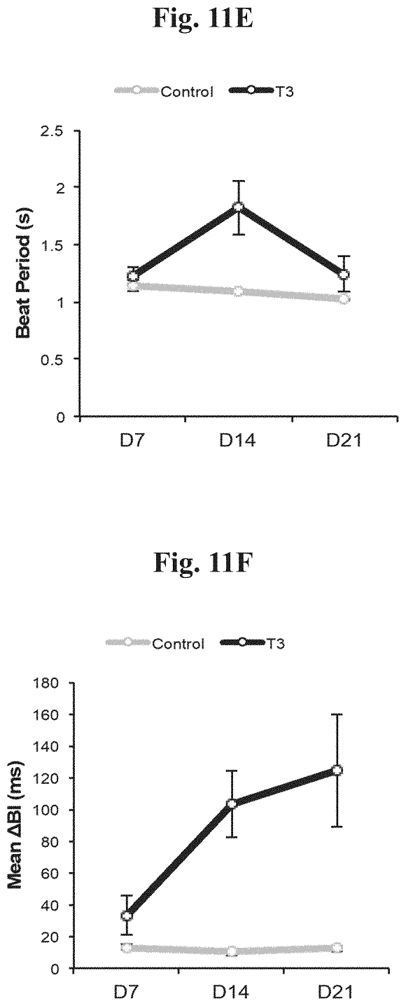

RNAseq was performed on cells exposed to all different combinations of the three maturation cues: NPs, T3, and Let7i. An empty vector (EV) negative control was included for virus exposure. For all other assays, the full ComboMat platform (Let7i+NP+T3) was compared to a viral vector Control (EV+Flat+No T3) and each maturation cue in isolation: NP (EV+NP+No T3), T3 (EV+Flat+T3), and Let7i (Let7i+Flat+No T3). The ComboMat platform utilizes nanotopographic substrate cues, thyroid hormone T3, and Let-7i microRNA (miR) overexpression at specific time-points during the culture period to induce advanced structural and phenotypic development of hPSC-CMs. Each cue was chosen to impact specific aspects of hPSC-CM development. Nanotopographic substrate cues have been shown to promote the structural maturation of primary and hPSC-CMs^{14,32}. These biophysical cues, however, have minimal impact on the metabolic development of hPSC-CMs. Therefore, thyroid hormone T3 was introduced as a biochemical cue to boost the metabolic, calcium handling, and contractile performance of the hPSC-CMs¹⁶. Preliminary results found, though, that long-term T3 exposure had a negative impact on sarcomere development and beat rate variability (FIG. 11), reminiscent of complications from hyperthyroidism. To counteract these detrimental effects, the transcriptome was manipulated using miRs such as the Let7 family. Along with improving metabolic capacity, hypertrophy, and the genetic profile″, Let7i helps to regulate the expression of the Hair Growth Associated gene that acts as a transcriptional co-repressor of many nuclear receptors, including the thyroid hormone receptor

hiPS Cell Line Generation and Maintenance

Normal urine-derived human induced pluripotent stem cells (hiPSCs) were obtained from an IRB-approved protocol as previously described“. Briefly, a polycistronic lentiviral vector encoding human Oct3/4, Sox2, Klf4, and c-Myc³³ was used to reprogram cells collected from clean-catch urine samples into iPSCs”. The derivative hiPSC line (UC3-4) was karyotyped and shown to be normal 46, XY karyotype. hiPSCs were cultured on Matrigel-coated tissue culture plastic, fed mTeSR culture medium (StemCell Technologies), and passaged using 1.9 U/mL Dispase (ThermoFisher). Cells were cultured in hypoxic conditions at 37° C., 5% CO₂, and 5% 02. DMD mutant hiPSCs (UC1015-6) were generated using CRISPR-Cas9 technology to create an isogenic pair from the Normal parental line UC3-4. Briefly, guide sequences targeting the first muscle specific exon of human dystrophin were used to delete a single G base at position 263 of the dystrophin gene to create a mutant allele (FIG. 7). Mutation of the dystrophin gene was confirmed via sequencing. DMD mutant hiPSCs were maintained in the same manner as the Normal UC3-4 parental line.

Cardiac Directed Differentiation and Cell Culture

A monolayer-based directed differentiation protocol was used as previously described²². hiPSCs were dissociated into single cells using Versene (ThermoFisher) and plated at a high density (250,000 cells/cm²) in mTeSR onto Matrigel-coated plates. Once a monolayer had formed, cells were treated with luM Chiron 99021 (Stemgent) in mTeSR until induction with 100 nM Activin-A (R&D Systems) in RPMI 1640 (ThermoFisher) supplemented with B27 minus insulin (RPMI/B27/Ins-) and a switch to normoxic conditions (37° C., 5% CO₂, ambient 02) at day 0. After 18 hours, cells were fed 5 ng/mL BMP4 (R&D Systems) plus 1 μM Chiron 99021 in RPMI/B27/Ins-medium. At day 3 post-induction, cells were fed 1 μM Xav 939 (Tocris) in RPMI/B27/Ins-medium. At day 5 post-induction, cells were fed fresh RPMI/B27/Ins-medium. At day 7 post-induction, cells were switched to RPMI-B27 medium containing insulin and medium (RPMI/B27/Ins+) was refreshed every other day. For T3 treated groups, 20 ng/ml T3 was freshly added to the RPMI/B27/Ins+.

Lentiviral Transduction of Let7i miRNA and Purification of Cardiomyocytes

The full description of the creation of the Let7i overexpression (OE) construct can be found in Kuppusamy et al.¹⁷. The control vector to Let7i was an eGFP expression pLKO.1 TRC vector (Addgene) under the U6 promoter. An eGFP construct was cloned between Agel and EcroRl sites of the pLKO.1 TRC vector. To generate lentiviral particles, 293FT cells were plated one day before transfection. On the day of transfection, 293FT cells were co-transfected with psPAX2 (Addgene #12260), pMD2.G (Addgene #12259), vector and polyethylenimine (PEI). Medium was changed 24 hours later and the lentivirus particles were then harvested 24 and 48 hours later. Lentiviral particles were concentrated using PEG-it Virus Precipitation Solution (5×) (System Biosciences).

At day 15 post-induction, beating monolayers of hiPSC-CMs were dissociated with 10 U/mL collagenase type I in DPBS containing calcium and 10 U/mL DNAse for 1 hour at 37° C. Cells were then collected, spun down, and resuspended in TrypLE (ThermoFisher) containing 10 U/mL DNAse for 5 minutes. A P1000 micropipette was used to triturate clumps of hiPSC-CMs in the TrypLE until single cells were obtained. Cells were then plated onto Matrigel-coated plated at 100,000 cells/cm². The following day, viral transduction of the cells was performed by diluting virus in the presence of hexadimethrine bormide (Polybrene, 6 μg/ml) in RPMI/B27/Ins+medium overnight. Cells were washed with PBS the following day and RPMI/B27/Ins+ was replaced.

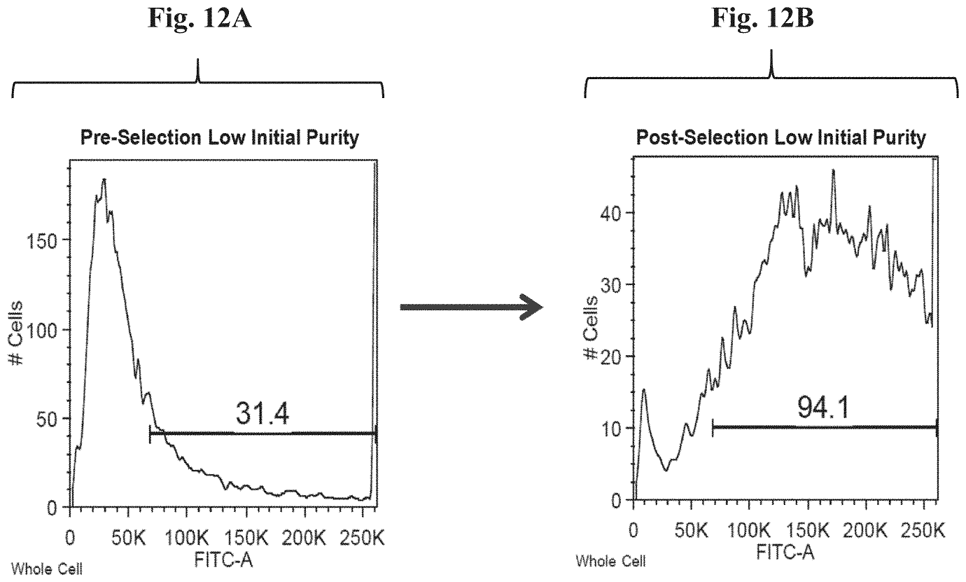

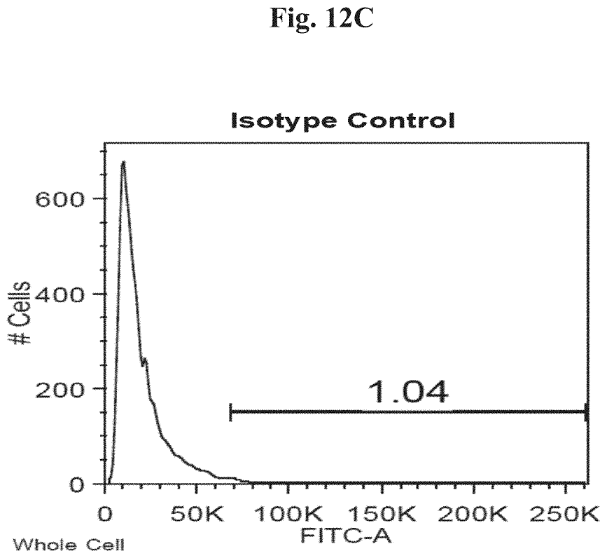

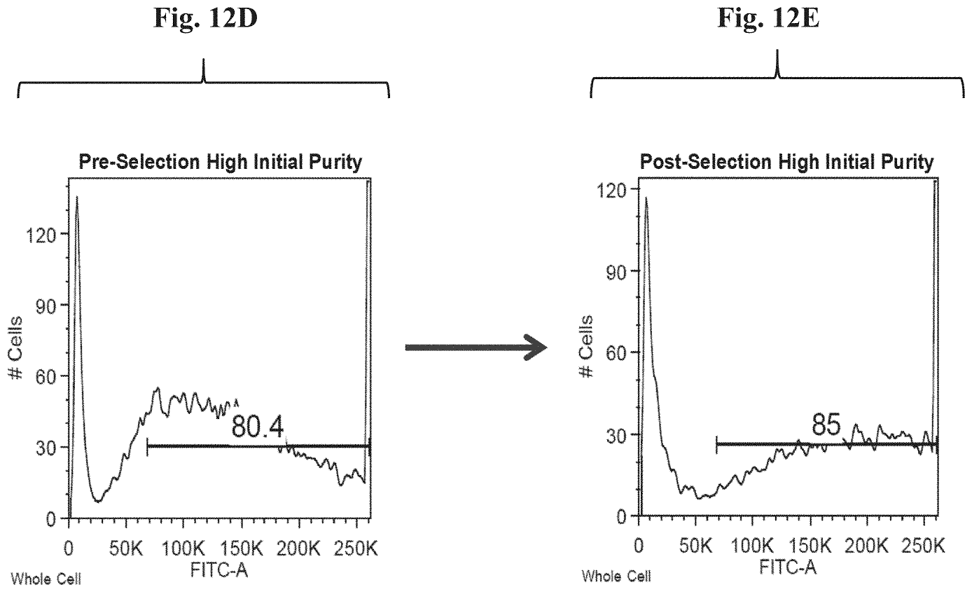

Cardiomyocytes were then purified via metabolic challenge³⁴ by feeding cells DMEM without glucose, sodium pyruvate, or glutamine (ThermoFisher) containing 2% horse serum and 4 mM lactate for three days without media change. Flow cytometry for cardiac purity was performed to confirm high purity samples (FIG. 12). Afterward, hiPSC-CMs were switched back to RPMI/B27/Ins+ media containing 2 μg/mL puromycin dihydrochloride for three days to select for successfully transduced hiPSC-CMs.

Fabrication of Nanopatterned Substrates and nanoMEAs

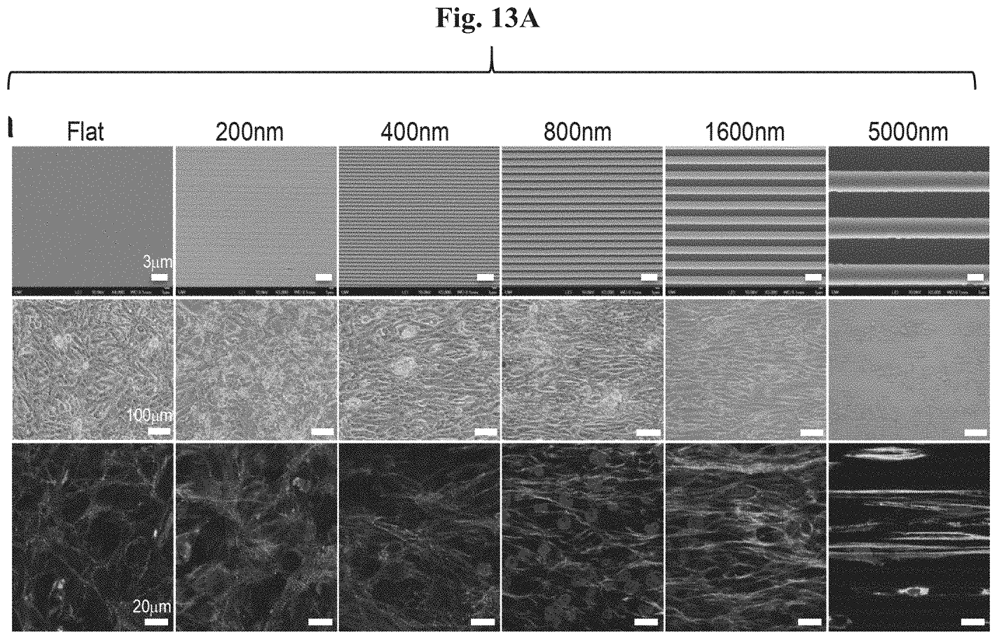

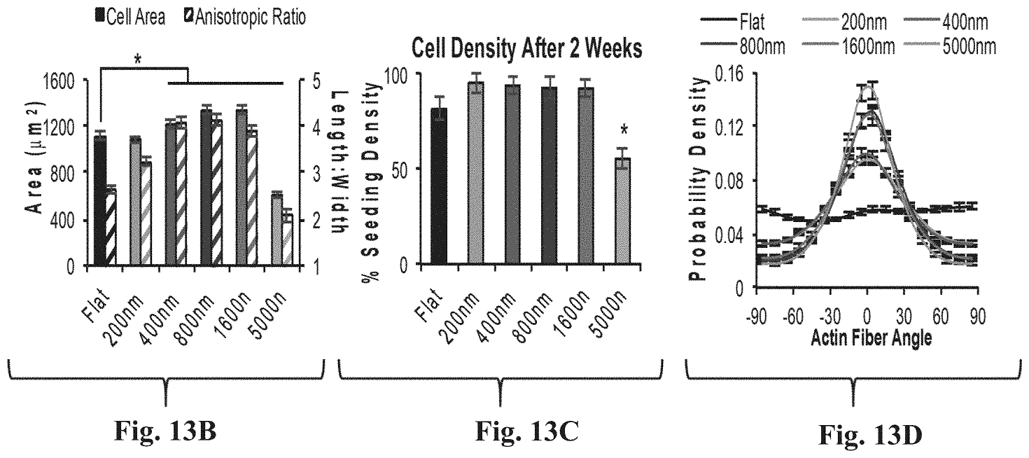

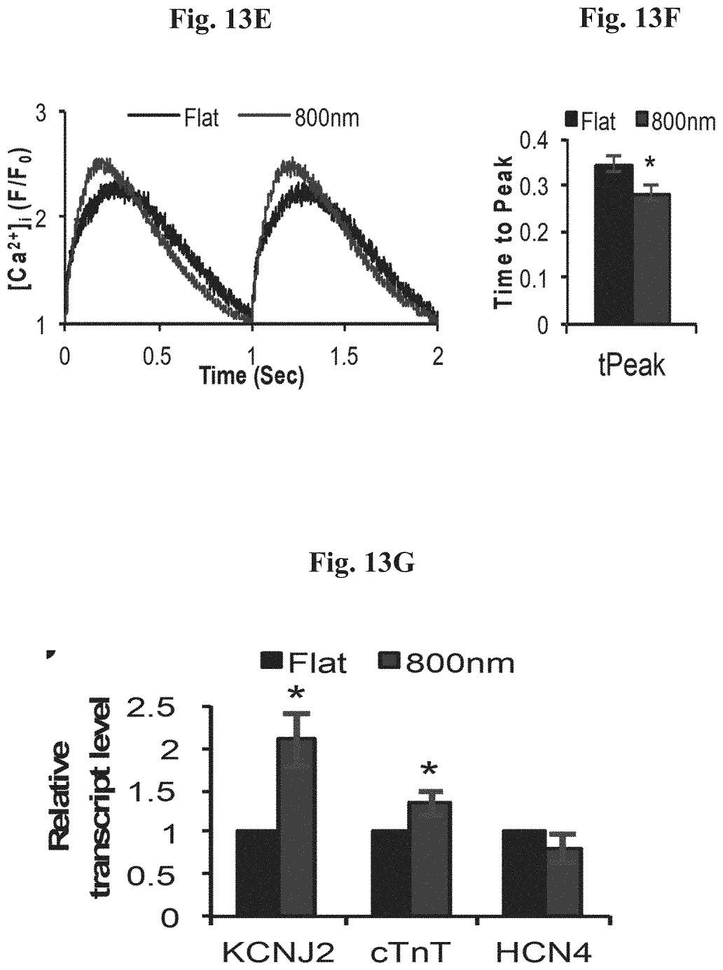

Nanopatterned substrates were fabricated via capillary force lithography as previously described³⁵. Polyurethane acrylate (PUA) master molds were fabricated by drop dispensing liquid PUA pre-polymer onto a deep-ion etched silicon master with the intended nanotopographic geometries (800 nm wide ridges and grooves, with 600 nm depth) and placing a transparent polyester (PET) film on top. The nanopattern dimensions were chosen to enhance the structure and function of hPSC-CMs (FIG. 13) After curing under a high-powered UV light source, the PUA and PET film were peeled from the silicon master to create a PUA master mold. To create the nanopatterned cell culture substrates, a polyurethane-based UV-curable polymer (NOA76, Norland Optical Adhesive) was drop dispensed onto glass primer treated 18 mm, #1 coverslips (Fisher Scientific) and the PUA master mold was placed on top. After curing with a UV light source, the PUA master mold was peeled away, leaving behind the nanopatterned surface (NPs). Control flat substrates were fabricated using the same process but replacing the nanopatterned PUA master with an unpatterned PET film.

To prepare surfaces for cell culture, PU-based NPs and control flat substrates were washed with 70% ethanol, oxygen plasma treated at 50 W for 5 minutes, placed under a UV-C light source for 1 hour to sterilize, then incubated for 24 hours at 37° C. with 5 μg/cm² human fibronectin (Life Technologies). hPSC-CMs were plated at a density of 150,000 cells/cm².

Nanopatterned MEAs (nanoMEAs) were fabricated as described in US Pat No. 20160017268A1. Briefly, the ion permeable polymer Nafion (Sigma-Aldrich) was patterned onto commercial multi-well MEA plates (Axion Biosystems) via solvent-mediated capillary force lithography. Prior to cell seeding, control flat or nanoMEA Nafion surfaces were sterilized with UV-C light and then incubated for 6 hours at 37° C. with 5 μg/cm² human fibronectin (Life Technologies). hPSC-CMs were plated in droplet fashion at 25,000 cells per well of the MEA plate.

Immunofluorescent Imaging and Morphological Analysis

After three weeks of the ComboMat platform, cells were prepared for immunofluorescent analysis by fixing in 4% paraformaldehyde (Affymetrix) for 15 minutes at room temperature. Samples were then permeabilized with 0.1% Triton X-100 (Sigma-Aldrich) in PBS and blocked with 5% goat serum. Cells were then incubated with mouse anti-α-actinin monoclonal antibody (1:1000, Sigma-Aldrich) in 1% goat serum overnight at 4° C. After washing with PBS, the samples were stained with Alexa-594 conjugated goat-anti-mouse secondary antibody (1:200, Invitrogen) as well as Alexafluo 488 labeled Phalloidin (1:200, Invitrogen). Nuclei counterstaining was performed with Vectashield containing DAPI (Vector Labs). A Nikon AIR confocal microscope with a Nikon CFI Plan Apo VC 60× water immersion objective was used to capture detailed immunofluorescent images for sarcomere analysis while a 20× air objective was used to obtain low-powered images of cells for measuring cell binucleation percentage. Cells from both objective powers were used to measure cell size. For morphological and sarcomere analysis, hPSC-CMs were plated at 10,000 cells/cm². For each condition, at least three biological specimens were plated with at least 7 cells imaged per specimen.

Quantitative Sarcomere Analysis

Immunofluorescent images were quantified for sarcomere alignment, pattern strength, and sarcomere length using a scanning gradient/Fourier transform script in MATLAB (Mathworks). Each image was broken into several small segments which were individually analyzed. Using a directional derivative, the image gradient for each segment was calculated to determine the local alignment of sarcomeres. The pattern strength was then determined by calculating the maximum peaks of one-dimensional Fourier transforms in the direction of the gradient. The lengths of sarcomeres were calculated by measuring the intensity profiles of the sarcomeres along this same gradient direction. The frequency at which the intensity profiles crossed their mean allowed an accurate calculation of local sarcomere length within each image segment. Once completed, this analysis allowed for unbiased subcellular-resolution mapping of sarcomere patterning, even in cells lacking the alignment cues provided by nanotopography.

Calcium Imaging

Intracellular calcium content was measured using the ratiometric indicator dye fura2-AM as described previously³⁶. In brief, cells were incubated in 0.2 μM fura2-AM dye in Tyrode's solution for 20 minutes at 37° C. and washed with PBS. Spontaneous Ca²⁺ transients were then monitored with the lonoptix Stepper Switch system coupled to a Nikon inverted fluorescence microscope. The fluorescence signal was acquired using a 40× Olympus objective and passed through a 510 nm filter, and the signal was quantified using a photomultiplier tube. Diastolic resting calcium concentration was quantified in the IonOptix software IonWizard using the formula:

$$[ {Ca}^{2 +} ] = {K}_{d} * \frac{R - {R}_{m t n}}{{R}_{max} - R} * \frac{S f 2}{S b 2} ,$$

where
K_{d}=fura2 calcium dissociation constant=225 nM
Rₘₐₓ and Rₘᵢₙ=ratio values measured under saturating calcium levels and in the absence of calcium, respectively.
Sf2/Sb2=ratio between the background subtracted wavelength 2 excited fluorescence in zero and saturating calcium solutions, respectively.

Electron Microscopy and Ultrastructural Analysis

After three weeks under the ComboMat platform, hiPSC-CMs were fixed in 4% glutaraldehyde in sodium cacodylate buffer for 2 hours at room temperature for transmission electron microscopy (TEM) analysis. Samples were then washed in buffer and stained in buffered 1% osmium tetroxide for 30 minutes on ice. Next, samples were washed in water and dehydrated in a graded series of ethanol. Samples were then treated by infiltration of 1:1 ethanol:Epon-Araldite epoxy resin followed by two changes of pure Epon-Araldite. At this point, a second coverslip was placed on top of the samples in a 60° C. oven for polymerization. The following day, the coverslips were dissolved using hydrofluoric acid and the samples were mounted onto blocks for sectioning Ultra-thin (70-80 nm) axial sections of single cells were contrasted with lead citrate prior to imaging with a JEOL JEM 1200EXII at 80 kV. ImageJ (National Institutes of Health) was used to measure Z-band width as defined as the width of contiguous electron-dense bands within a sarcomere structure.

MEA Electrophysiological Analysis

Electrophysiological analysis of spontaneously beating cardiomyocytes was collected for 2 minutes using the AxIS software (Axion Biosystems). After raw data collection, the signal was filtered using a Butterworth band-pass filter and a 90 μV spike detection threshold. Field potential duration was automatically determined using a polynomial fit T-wave detected algorithm. Beat interval analysis on 30 consecutive beats was performed as previously published²⁶ (see Table 3).

TABLE 3
Beat Interval Analysis Parameters.

| Parameter | Description |
|---|---|
| StDev BI | The standard deviation of beat intervals. |
| Mean ΔBI | The mean absolute value of the difference |
| | between consecutive beats. |
| Median ΔBI | The median absolute value of the differences |
| | between consecutive beats. |
| StDev ΔBI | The standard deviation of the absolute value |
| | of the difference between consecutive beats. |
| % ΔBI > 100 ms | The percentage of absolute ΔBI values |
| | exceeding 100 ms. |
| % ΔBI > 250 ms | The percentage of absolute ΔBI values |
| | exceeding 250 ms. |

Mitochondrial Functional Assay

Cellular metabolic function was measured using the Seahorse XF96 extracellular flux analyzer as previously published¹⁷. After three weeks of treatment with the ComboMat platform, cells were trypsinized and replated onto fibronectin coated (5 μg/cm²) XF96 plates at a density of 30,000 cells per well. The MitoStress and Palmitate assays were performed three days post-replating onto the XF96 plates. One hour prior to experiments, RPMI/B27/Ins+ media was replaced with Seahorse XF Assay media supplemented with sodium pyruvate (Gibco/Invitrogen, 1 mM) and 25 mM glucose for the MitoStress assay or 25 mM glucose with 0.5 mM Carnitine for the Palmitate assay. For the MitoStress assay, injection of substrates and inhibitors were applied during the measurements to achieve final concentrations of 4-(trifluoromethoxy) phenylhydrazone at 1 (FCCP; Seahorse Biosciences), oligomycin (2.5 μM), antimycin (2.504) and rotenone (2.5 μM). For the Palmitate assay, injection of substrates and inhibitors were applied during the measurements to achieve final concentrations of 200 mM palmitate or 3304 BSA. The OCR values were further normalized to the number of cells present in each well, quantified by the Hoechst staining (Hoechst 33342; Sigma-Aldrich) as measured using fluorescence at 355 nm excitation and 460 nm emission. Basal respiration in the MitoStress assay was defined as the OCR prior to oligomycin addition while maximum respiratory capacity was defined as the change in OCR in response to FCCP from the OCR after the addition of oligomycin. Exogenous fatty acid utilization was defined as the maximum change in OCR from baseline after the addition of Palmitate.

Contraction Analysis

A video-based contraction analysis algorithm was used, termed Correlation-based Contraction Quantification (CCQ), utilizes particle image velocimetry and digital image correlation algorithms³⁷ to track the motion of cells from bright field video recordings as previously published²². Functional outputs such as contraction magnitude, velocity, and angle are output as a vector field for each video frame and can be averaged spatially or temporally. In brief, a reference frame is divided into a grid of windows of a set size. To track motion, each window is run through a correlation scheme comparing a second frame, providing the location of that window in the second frame. The correlation equation used provided a Gaussian correlation peak with a probabilistic nature that provides sub-pixel accuracy. The videos used for this analysis were taken with a Nikon camera at 20× magnification and 30 frames per second.

RNA-Seq Analysis

Total RNA was extracted using trizol (Thermo Fisher Scientific) from RUES2 hESC-CMs. RNA-seq samples were aligned to hg19 using Tophat (PMID: 19289445, version 2.0.13). Gene-level read counts were quantified using htseq-count (PMID: 25260700) using Ensembl GRCh37 gene annotations. Genes with total expression above 3 RPKM summed across RNA-seq samples were kept for further analysis. princomp function from R was used to for Principal Component Analysis. DESeq (PMID: 20979621) was used for differential gene expression analysis. Genes with fold change >1.5 were considered differentially expressed. topGO R package (PMID: 16606683) was used for Gene Ontology enrichment analysis.

For the heat map of pathway enrichment, each condition was compared against the Control (EV-Flat) condition, and up-regulated genes (>1.5 fold change) and down-regulated genes were identified (<−1.5 fold change). Hypergeometric test was performed on up- and down-regulated genes separately for enrichment against a curated set of pathways that are beneficial for cardiac maturation, resulting in a m by n matrix, where m is the number of pathways (m=7) and n is the number of conditions (n=10). The negative log 10 of the ratio between enrichment p-value for up- and down-regulated genes were calculated to represent the overall net “benefit” of a treatment: large positive value (>0) means the treatment results in more up-regulation of genes in cardiac maturation pathways than down-regulation of these genes, and more negative values means the treatment results in more down-regulation of genes in cardiac maturation pathways.

Flow Cytometry

Cells were dissociated and prepared for flow cytometry before and after lactate purification and puromycin selection to determine selection efficiency. Cells were first fixed in 4% paraformaldehyde for 15 minutes. Cells were then permeabilized with 0.75% Saponin and stained in PBS containing 5% FBS with either 1:100 mouse anti-cTnT or isotype control mouse IgG antibodies. Alexa-488 conjugated goat-anti-mouse secondary antibody (1:200, Invitrogen) was used for visualization. Samples were run on a BD Sciences FACS Canto flow cytometer and analyzed with FlowJo software.

High-Throughput Drug Screening

Cardiomyocytes were dissociated 14 days post differentiation into a single cell suspension by 5 minute incubation in TrypLE (Life Technologies) and plated at 10,000 cells per well on opaque-bottom 384-well plates (Nunc) pre-coated with 1 mg/mL Matrigel (Corning) for one hour at 37° C. Cells were then cultured on the 384-well plates for an additional 16 days, with media exchanges every 72 hours. After 15 days, compounds dissolved in DMSO were distributed to the plates using the CyBi Well Vario 384/25 liquid handler (Cybio, Germany) to achieve concentrations of 10⁻⁸, 10⁻⁷, 10⁻⁶, 10⁻⁵ M in duplicate. After 18 hours of incubation, medium was aspirated and pure water was injected to reach 12.5% normal osmolarity using BioTek EL406 Washer Dispenser (BioTek, Winooski, Vt.). After 30 minutes incubation, supernatants were removed and plates were assayed by adding CellTiter Glo (Promega) according to the manufacturer's instructions. After 5 minutes, the luminescence was measured using EnVision Multilable Resaer (PerkinElmer). All data were processed and visualized by Tibco Spotfire (Tibco Spotfire, Boston, Mass.). Percentage viability was calculated by comparing signal from each well to the average of the control wells treated with DMSO alone (32 control wells per plate).

Statistical Analyses

Statistical significance between groups was determined using either a One-Way or Two-Way ANOVA with Tukey's pairwise post hoc analysis from SigmaPlot software unless otherwise stated. For all statistical analyses, a p-value less than 0.05 was considered significant. Error bars represent standard error of the mean (SEM).

### REFERENCES

1. Celermajer, D. S., Chow, C. K., Marijon, E., Anstey, N. M. & Woo, K. S. Cardiovascular Disease in the Developing World. JAC 60, 1207-1216 (2012).
2. Watkins, H., Ashrafian, H. & Redwood, C. Inherited Cardiomyopathies. The New England Journal of Medicine 364, 1-14 (2011).
3. Kim, C. iPSC technology-Powerful hand for disease modeling and therapeutic screen. BMB Reports 48, 256-265 (2015).
4. Yang, X., Pabon, L. & Murry, C. E. Engineering Adolescence: Maturation of Human Pluripotent Stem Cell-Derived Cardiomyocytes. Circulation Research 114, 511-523 (2014).
5. Moretti, A. et al. Patient-Specific Induced Pluripotent Stem-Cell Models for Long-QT Syndrome. N Engl J Med 363, 1397-1409 (2010).
6. Itzhaki, I. et al. Modelling the long QT syndrome with induced pluripotent stem cells. Nature 471, 225-229 (2012).
7. Novak, A. et al. Cardiomyocytes generated from CPVT. J. Cell. Mol. Med. 16, 468-482 (2012).
8. Huang, H.-P. et al. Human Pompe disease-induced pluripotent stem cells for pathogenesis modeling, drug testing and disease marker identification. Human Molecular Genetics 20, 1-14 (2011).
9. Lan, F. et al. Abnormal calcium handling properties underlie familial hypertrophic cardiomyopathy pathology in patient-specific induced pluripotent stem cells. Cell Stem Cell 12, 101-113 (2013).
10. Sun, N. et al. Patient-specific induced pluripotent stem cells as a model for familial dilated cardiomyopathy. Science Translational Medicine 4, 130ra47-130ra47 (2012).
11. Guan, X. et al. Dystrophin-deficient cardiomyocytes derived from human urine: New biologic reagents for drug discovery. Stem Cell Research 12, 467-480 (2014).
12. Lin, B. et al. Modeling and study of the mechanism of dilated cardiomyopathy using induced pluripotent stem cells derived from individuals with Duchenne muscular dystrophy. Disease Models & Mechanisms 8, 457-466 (2015).
13. Smith, A. S. T., Macadangdang, J., Leung, W., Laflamme, M. A. & Kim, D.-H. Human iPSC-derived cardiomyocytes and tissue engineering strategies for disease modeling and drug screening. Biotechnology Advances 35, 77-94 (2017).
14. Carson, D. et al. Nanotopography-Induced Structural Anisotropy and Sarcomere Development in Human Cardiomyocytes Derived from Induced Pluripotent Stem Cells. ACS Appl. Mater. Interfaces 8, 21923-21932 (2016).
15. Shiraishi, I., Takamatsu, T. & Fujita, S. Three-dimensional observation with a confocal scanning laser microscope of fibronectin immunolabling during cardiac looping in the chick embryo. Anatomy and Embryology 191, 1-8 (1995).
16. Yang, X. et al. Tri-iodo-1-thyronine promotes the maturation of human cardiomyocytes-derived from induced pluripotent stem cells. Journal of Molecular and Cellular Cardiology 72, 296-304 (2014).
17. Kuppusamy, K. T. et al. Let-7 family of microRNA is required for maturation and adult-like metabolism in stem cell-derived cardiomyocytes. Proceedings of the National Academy of Sciences 112, E2785-E2794 (2015).
18. Finsterer, J. & Cripe, L. Treatment of dystrophin cardiomyopathies. 11, 168-179 (2014).
19. Mourkioti, F. et al. Role of telomere dysfunction in cardiac failure in Duchenne muscular dystrophy. 15, 895-904 (2013).
20. Leung, D. G. et al. Sildenafil does not improve cardiomyopathy in Duchenne/Becker muscular dystrophy. Ann Neurol. 76, 541-549 (2014).
21. Adamo, C. M. et al. Sildenafil reverses cardiac dysfunction in the mdx mouse model of Duchenne muscular dystrophy. Proceedings of the National Academy of Sciences 107, 19079-19083 (2010).
22. Macadangdang, J. et al. Nanopatterned Human iPSC-Based Model of a Dystrophin-Null Cardiomyopathic Phenotype. Cellular and Molecular Bioengineering 8, 320-332 (2015).
23. Young, C. S. et al. A Single CRISPR-Cas9 Deletion Strategy that Targets the Majority of DMD Patients Restores Dystrophin Function in hiPSC-Derived Muscle Cells. 1-9 (2016). doi:10.1016/j.stem.2016.01.021
24. Gurvich, O. L. et al. DMDexon 1 truncating point mutations: Amelioration of phenotype by alternative translation initiation in exon 6. Hum. Mutat. 30, 633-640 (2009).
25. Pei, H., Yao, Y., Yang, Y., Liao, K. & Wu, J.-R. Kruppel-like factor KLF9 regulates PPARgamma transactivation at the middle stage of adipogenesis. Cell Death and Differentiation 18, 315-327 (2010).
26. Gilchrist, K. H., Lewis, G. F., Gay, E. A., Sellgren, K. L. & Grego, S. High-throughput cardiac safety evaluation and multi-parameter arrhythmia profiling of cardiomyocytes using microelectrode arrays. Toxicology and Applied Pharmacology 288, 249-257 (2015).
27. Vassalle, M. & Lin, C.-I. Calcium Overload and Cardiac Function. J Biomed Sci 11, 542-565 (2004).
28. Thandroyen, F. T. et al. Intracellular calcium transients and arrhythmia in isolated heart cells. Circulation Research 69, 810-819 (1991).
29. van Westering, T., Betts, C. & Wood, M. Current Understanding of Molecular Pathology and Treatment of Cardiomyopathy in Duchenne Muscular Dystrophy. Molecules 2015, Vol. 20, Pages 8823-8855 20, 8823-8855 (2015).
30. Wen, J.-Y. et al. Maturation-Based Model of Arrhythmogenic Right Ventricular Dysplasia Using Patient-Specific Induced Pluripotent Stem Cells. Circ J 1-7 (2015). doi:10.1253/circj.CJ-15-0363
31. Tiburcy, M. et al. Defined Engineered Human Myocardium With Advanced Maturation for Applications in Heart Failure Modeling and RepairClinical Perspective. Circulation 135, 1832-1847 (2017).
32. Kim, D.-H. et al. Nanoscale cues regulate the structure and function of macroscopic cardiac tissue constructs. Proceedings of the National Academy of Sciences 107, 565-570 (2010).
33. Warlich, E. et al. Lentiviral Vector Design and Imaging Approaches to Visualize the Early Stages of Cellular Reprogramming. Mol Ther 19, 782-789 (2009).
34. Tohyama, S. et al. Distinct Metabolic Flow EnablesLarge-Scale Purification of Mouse and Human Pluripotent Stem Cell-Derived Cardiomyocytes. Stem Cell 12, 127-137 (2013).
35. Macadangdang, J. et al. Capillary Force Lithography for Cardiac Tissue Engineering. e50039 (2014).
36. Lundy, S. D., Zhu, W.-Z., Regnier, M. & Laflamme, M. A. Structural and functional maturation of cardiomyocytes derived from human pluripotent stem cells. Stem Cells and Development 22, 1991-2002 (2013).
37. Milde, F. et al. Cell Image Velocimetry (CIV): boosting the automated quantification of cell migration in wound healing assays. Integr. Biol. 4, 1437 (2012).

SEQUENCES

SEQ ID NO: 1
CTGGCTGAGG TAGTAGTTTG TGCTGTTGGT CGGGTTGTGA CATTGCCCGC
TGTGGAGATAACTGCGCAAG CTACTGCCTT GCTA
SEQ ID NO: 2
CTGGCTGAGG TAGTAGTTTG TGCTGTTGGT CGGGTTGTGA CATTGCCCGC
TGTGGAGATAACTGCGCAAG CTACTGCCTT GCTAG
SEQ ID NO: 3
CTGGCTGAGG TAGTAGTTTG TGCTGTTGGT CGGGTTGTGA CATTGCCCGC
TGTGGAGATAACTGCGCAAG CTACTGCCTT GCT
SEQ ID NO: 4
GGGCCCTGGC TGAGGTAGTA GTTTGTGCTG TTGGTCGGGT TGTGACATTG
CCCGCTGTGGAGATAACTGC GCAAGCTACT GCCTTGCTAG TG
SEQ ID NO: 5
CUGGCUGAGGUAGUAGUUUGUGCUGUUGGUCGGGUUGUGACAUUGCCCGCUGU
GGAGAUAACUGCGCAAGCUACUGCCUUGCUAG
SEQ ID NO: 6
UGAGGUAGUAGUUUGUGCU
SEQ ID NO: 7
UCTCCUTCUTCUUUCUCGU
SEQ ID NO: 8 (Reverse Complement of SEQ ID NO: 6)
UGCUCUUUCTUCTUCCTCU
SEQ ID NO: 9 (Reverse Sequence of SEQ ID NO: 6)
UCGUGUUUGAUGAUGGAGU
SEQ ID NO: 10
UGAGGUAGUAGGUUGUAUAGUU
SEQ ID NO: 11
UGAGGUAGUAGGUUGUGUGGUU
SEQ ID NO: 12
UGAGGUAGUAGGUUGUAUGGUU
SEQ ID NO: 13
AGAGGUAGUAGGUUGCAUAGU
SEQ ID NO: 14
UGAGGUAGGAGGUUGUAUAGU
SEQ ID NO: 15
UGAGGUAGUAGAUUGUAUAGUU
SEQ ID NO: 16
UGAGGUAGUAGUUUGUACAGU
SEQ ID NO: 17
UGAGGUAGUAGUUUGUGCU
SEQ ID NO: 18
ATGCTTTGGTGGGAAGAAGTAGAGGACTGTT
SEQ ID NO: 19
ATGCTTTGGTGGGAAGAATAGAGGACTGTT

1. A method of making stem cell-derived cardiomyocytes, the method comprising, contacting stem cell derived cardiomyocytes with:
a. a nanopatterned substrate;
b. thyroid hormone T3; and
c. a Let7i microRNA.

2. The method of claim 1, wherein the nanopatterned substrate comprises a nanopatterned surface with a substantially parallel array of grooves and ridges.

3. The method of claim 2, wherein the dimensions of each groove or ridge are less than 1000 nanometers in length, width, or height.

4. The method of claim 2, wherein the grooves and ridges are 800 nm wide, and the grooves are 600 nm deep.

5. The method of claim 1, wherein Let7i microRNA is expressed by the stem cell-derived cardiomyocyte.

6. The method of claim 1, wherein the step of contacting the cardiomyocytes with a Let7i microRNA comprises contacting the stem cell-derived cardiomyocytes with a viral vector.

7. The method of claim 1, wherein the stem cell-derived cardiomyocytes are human cardiomyocytes.

8. The method of claim 1, wherein the stem cell-derived cardiomyocytes are differentiated from an induced pluripotent stem cell (iPS cell) or an embryonic stem cell.

9. The method of claim 1, wherein the stem cell-derived cardiomyocytes are derived from a subject with a muscular disease or disorder.

10. The method of claim 1, wherein the stem cell-derived cardiomyocytes are genetically modified.

11. The method of claim 10, wherein the cardiomyocytes are contacted with nanopatterned substrate and thyroid hormone T3 after contacting with a vector encoding a Let7i microRNA.

12. The method of claim 1, wherein the resulting stem cell-derived cardiomyocytes have a more mature cardiomyocyte phenotype when compared with the stem cell-derived cardiomyocytes prior to contacting with the nanopatterned substrate, thyroid hormone T3, and Let7i microRNA.

13. The method of claim 1, wherein the nanopatterned substrate comprises a microelectrode array that permits electrical stimulation and/or measurement of cardiomyocyte electrophysiological properties.

14. A method of maturing stem cell-derived cardiomyocytes, the method comprising contacting stem cell-derived cardiomyocytes with:
a. a nanopatterned substrate;
b. thyroid hormone T3; and
c. a Let7i microRNA.

15. The method of claim 14, wherein the nanopatterned substrate comprises a nanopatterned surface with a substantially parallel array of grooves and ridges.

16. The method of claim 15, wherein the dimensions of each groove or ridge are less than 1000 nanometers in length, width, or height.

17. The method of claim 15, wherein the grooves and ridges are 800 nm wide, and the grooves are 600 nm deep.

18. The method of claim 14, wherein Let7i microRNA is expressed by the stem cell-derived cardiomyocyte.

19. The method of claim 14, wherein the step of contacting the cardiomyocytes with a Let7i microRNA comprises contacting the stem cell-derived cardiomyocytes with a viral vector.

20. The method of claim 14, wherein the stem cell-derived cardiomyocytes are human cardiomyocytes.

21. The method of claim 14, wherein the stem cell-derived cardiomyocytes are differentiated from an induced pluripotent stem cell (iPS cell) or an embryonic stem cell.

22. The method of claim 14, wherein the stem cell-derived cardiomyocytes are derived from a subject with a muscular disease or disorder.

23. The method of claim 14, wherein the stem cell-derived cardiomyocytes are genetically modified.

24. The method of claim 23, wherein the cardiomyocytes are contacted with the nanopatterned substrate and thyroid hormone T3 after contacting with a vector encoding a Let7i microRNA.

25. The method of claim 14, wherein the resulting stem cell-derived cardiomyocytes have a more mature cardiomyocyte phenotype when compared with the stem cell-derived cardiomyocytes prior to contacting with the nanopatterned substrate, thyroid hormone T3, and Let7i microRNA.

26. The method of claim 14, wherein the nanopatterned substrate comprises a microelectrode array that permits electrical stimulation and/or measurement of cardiomyocyte electrophysiological properties.

27. A method of evaluating cardiotoxicity of an agent, the method comprising contacting stem cell-derived cardiomyocytes prepared by the method of claim 1 or claim 14 with the agent.

28. The method of claim 27, further comprising detecting at least one phenotypic characteristic of the cardiomyocytes.

29. The method of claim 27, wherein the agent is selected from the group consisting of a small molecule, an antibody, a peptide, a genome editing system and a nucleic acid.

30. The method of claim 27, wherein cardiotoxicity of an agent is indicated by the agent's effect on one or more of: cell viability, cell size, sarcomere length, organization of sarcomeres within a tissue, a biopotential or electrical property of a population of stem cell-derived cardiomyocytes, mitochondrial function, gene expression, beat rate, beat strength, and contractility.

31. An assay for identifying an agent that modulates a functional property of a cardiomyocyte, the assay comprising:
a. contacting a population of stem cell-derived cardiomyocytes prepared by the method of claim 1 or claim 14 with a candidate agent; and
b. detecting at least one functional property of the cardiomyocytes,
wherein detecting a change in at least one functional property of the cardiomyocytes after contacting step (a) identifies the agent as one that can modulate a functional property of a cardiomyocyte.

32. The assay of claim 31, wherein detecting step (b) comprises detecting at least one of the following properties: cell viability, cell size, sarcomere length, organization of sarcomeres within a tissue, a biopotential or electrical property, mitochondrial function, gene expression, beat rate, beat strength, and contractility.

33. A disease model comprising a stem cell-derived cardiomyocyte prepared by the method of claim 1 or claim 14, wherein the stem cell is derived from a subject with a muscular disease or disorder, or wherein the stem cell-derived cardiomyocyte or the stem cell from which it is derived is genetically modified such that the cardiomyocyte expresses a disease phenotype.

35. The disease model of claim 33, wherein the muscular disease or disorder has a phenotype with cardiac dysfunction.

36. The disease model of claim 33, wherein the muscular disease or disorder is characterized by adult onset of the cardiac phenotype.

37. The disease model of claim 33, wherein the muscular disease or disorder is Duchenne Muscular Dystrophy.

38. A composition comprising stem cell-derived cardiomyocytes on a nanopatterned substrate, the composition further comprising thyroid hormone T3 and a Let7i microRNA.

39. The composition of claim 37, wherein the stem cell-derived cardiomyocytes are derived from a subject with a muscular disease or disorder.

40. The composition of claim 37, wherein the cardiomyocytes are human cardiomyocytes.

41. The composition of claim 37, wherein the stem cell-derived cardiomyocytes, or the stem cells from which they are derived, are genetically modified such that the stem cell-derived cardiomyocytes exhibit a cardiac dysfunction phenotype.

42. A composition comprising cardiomyocytes made by contacting in vitro-differentiated cardiomyocytes with a nanopatterned substrate, thyroid hormone T3, and a Let7i microRNA wherein the cardiomyocytes have a more mature phenotype as compared with in vitro-differentiated cardiomyocytes that were not contacted with the nanopatterned substrate, thyroid hormone T3 and Let7i microRNA.

43. The composition of claim 41, wherein the cardiomyocytes are derived from a subject with a muscular disease or disorder.

44. The composition of claim 41, wherein the in vitro-differentiated cardiomyocytes or the stem cells from which they are differentiated are genetically modified such that they exhibit a cardiac dysfunction phenotype.

45. A kit comprising stem cell-derived cardiomyocytes, a nanopatterned substrate, thyroid hormone T3, a vector encoding a Let7i microRNA, and packaging materials therefor.

46. The kit of claim 44, further comprising cell culture medium and instructions to permit preparation of mature in vitro differentiated cardiomyocytes from the stem cell-derived cardiomyocytes.

47. The kit of claim 44, wherein the nanopatterned substrate comprises a nanopatterned surface with a substantially parallel array of grooves and ridges.

48. The kit of claim 46, wherein the dimensions of each groove or ridge are less than 1000 nanometers in length, width, or height.

49. The kit of claim 46, wherein the grooves and ridges are 800 nm wide, and the grooves are 600 nm deep.

50. The kit of claim 44, wherein the stem cell-derived cardiomyocytes are human.

51. The kit of claim 44, wherein the stem cell-derived cardiomyocytes are derived from a subject with a muscular disease or disorder.

52. The kit of claim 44, wherein the stem cell-derived cardiomyocytes are frozen.

53. A method of making stem cell-derived cardiomyocytes, the method comprising, contacting stem cell derived cardiomyocytes with:
a. a nanopatterned substrate comprising a substantially parallel array of grooves and ridges, wherein the grooves and ridges are 800 nm wide, and the grooves are 600 nm deep;
b. thyroid hormone T3; and
c. a Let7i microRNA.

54. A composition comprising stem cell-derived cardiomyocytes on a nanopatterned substrate, the composition further comprising:
a. a nanopatterned substrate comprising a substantially parallel array of grooves and ridges, wherein the grooves and ridges are 800 nm wide, and the grooves are 600 nm deep;
b. thyroid hormone T3; and
c. a Let7i microRNA.

55. The method of any one of claims 1-32 and 53, wherein the nanopatterned substrate comprises an elastomeric substrate that permits mechanical stimulation of the cardiomyocytes cultured thereupon, and wherein the method further comprises subjecting the cardiomyocytes to such mechanical stimulation.

56. The composition of any one of claim 38-41 or 54, or the kit of any one of claims 45-52, wherein the nanopatterned substrate comprises an elastomeric substrate that permits mechanical stimulation of cardiomyocytes cultured thereupon.

57. The kit of any one of claims 43-51, wherein the cardiomyocytes are on the nanopatterned substrate.

58. The kit of any one of claims 43-51 and 57, which permits shipping at a temperature between room temperature and 4° C.


SEQUENCE LISTING (24 sequences)

SEQ ID NO: 1 - DNA, 84 bp, Artificial Sequence
  Description of Artificial Sequence Synthetic oligonucleotide
ctggctgaggtagtagtttgtgctgttggtcgggttgtgacattgcccgctgtggagataactgcgcaagctactgccttgcta

SEQ ID NO: 2 - DNA, 85 bp, Artificial Sequence
  Description of Artificial Sequence Synthetic oligonucleotide
ctggctgaggtagtagtttgtgctgttggtcgggttgtgacattgcccgctgtggagataactgcgcaagctactgccttgctag

SEQ ID NO: 3 - DNA, 83 bp, Artificial Sequence
  Description of Artificial Sequence Synthetic oligonucleotide
ctggctgaggtagtagtttgtgctgttggtcgggttgtgacattgcccgctgtggagataactgcgcaagctactgccttgct

SEQ ID NO: 4 - DNA, 92 bp, Artificial Sequence
  Description of Artificial Sequence Synthetic oligonucleotide
gggccctggctgaggtagtagtttgtgctgttggtcgggttgtgacattgcccgctgtggagataactgcgcaagctactgccttgctagtg

SEQ ID NO: 5 - RNA, 85 nt, Homo sapiens
cuggcugagguaguaguuugugcuguuggucggguugugacauugcccgcuguggagauaacugcgcaagcuacugccuugcuag

SEQ ID NO: 6 - RNA, 19 nt, Homo sapiens
ugagguaguaguuugugcu

SEQ ID NO: 7 - DNA, 19 bp, Artificial Sequence
  Description of Artificial Sequence Synthetic oligonucleotide
  Description of Combined DNA/RNA Molecule Synthetic oligonucleotide
uctccutcutcuuucucgu

SEQ ID NO: 8 - DNA, 19 bp, Artificial Sequence
  Description of Artificial Sequence Synthetic oligonucleotide
  Description of Combined DNA/RNA Molecule Synthetic oligonucleotide
ugcucuuuctuctucctcu

SEQ ID NO: 9 - RNA, 19 nt, Homo sapiens
ucguguuugaugauggagu

SEQ ID NO: 10 - RNA, 22 nt, Homo sapiens
ugagguaguagguuguauaguu

SEQ ID NO: 11 - RNA, 22 nt, Homo sapiens
ugagguaguagguugugugguu

SEQ ID NO: 12 - RNA, 22 nt, Homo sapiens
ugagguaguagguuguaugguu

SEQ ID NO: 13 - RNA, 21 nt, Homo sapiens
agagguaguagguugcauagu

SEQ ID NO: 14 - RNA, 21 nt, Homo sapiens
ugagguaggagguuguauagu

SEQ ID NO: 15 - RNA, 22 nt, Homo sapiens
ugagguaguagauuguauaguu

SEQ ID NO: 16 - RNA, 21 nt, Homo sapiens
ugagguaguaguuuguacagu

SEQ ID NO: 17 - RNA, 19 nt, Homo sapiens
ugagguaguaguuugugcu

SEQ ID NO: 18 - DNA, 31 bp, Homo sapiens
atgctttggtgggaagaagtagaggactgtt

SEQ ID NO: 19 - DNA, 30 bp, Artificial Sequence
  Description of Artificial Sequence Synthetic oligonucleotide
atgctttggtgggaagaatagaggactgtt

SEQ ID NO: 20 - RNA, 16 nt, Homo sapiens
ugagguaguaguuugu

SEQ ID NO: 21 - DNA, 53 bp, Homo sapiens
tttcaaaatgctttggtgggaagaagtagaggactgttatgaaagagaagatg

SEQ ID NO: 22 - DNA, 52 bp, Artificial Sequence
  Description of Artificial Sequence Synthetic oligonucleotide
tttcaaaatgctttggtgggaagaatagaggactgttatgaaagagaagatg

SEQ ID NO: 23 - protein, 10 aa, Homo sapiens
MLWWEEVEDC

SEQ ID NO: 24 - protein, 6 aa, Artificial Sequence
  Description of Artificial Sequence Synthetic peptide
MLWWEE